SEQUENCE LISTING (2 sequences)

SEQ ID NO: 1 - DNA, 2304 bp, Human - Parkinson′s 3247
gaattcgtccaaactgaggatcacaagtctccacattctgagtaggaggatgagggtctgagttaggatttgggtcctgcagggcttgctaaggaatcccctgatggcctaggattccacgcagagcacatctggtgtgagagagctcgctgcaagggtgaaggctccgccctatcagatagacaaccaggccaccaagaggcccagccctccaaaccctggatttgcaacatcctcaaagaacagcaacgggccttgagcagaattgagaaggaaatacccccacctgccctcagccgttaagtgggctttgctattcacaagggcctctgggtgtcctggcagagaggggagatggcacaggcaccaggtgctagggtgccagggcctcccgagaaggaacaggtgcaaagcaggcaattagcccagaaggtatccgtggggcaggcagcctagatctgatgggggaagccaccaggattacatcatctgctgtaacaactgctctgaaaagaagatatttttcaacctgaacttgcagtagctagtggagaggcaggaaaaaggaaatgaaacagagacagagggaagcctgagccaaaatagaccttcccgagagaggaggaagcccggagagagacgcacggtcccctccccgcccctaggccgccgccccctctctgccctcggcggcgagcagggcgccgcgacccggggccggaaaggtgccaggggctccgggcggccgggcgggcgcacaccatccccgcgggcggcgcggagccggcgacagcgcgcgagagggaccgggcggtggcggcggcgggaccgggatggaagggagcgcggtgactgtccttgagcgcggaggggcgagctcgccggcggaggccgagcaagcggaggcaggagcggcggcgacggcggcggcggcggcggcgcccgagcacccgagggggtccgagccccggcagccggccagccccgcgccacaaagggagcgcccccgccgcccggcaccccgcctccctccccaatgtcctcggccatcgaaaggaagagcctggacccttcagaggaaccagtggatgaggtgctgcagatccccccatccctgctgacatgcggcggctgccagcagaacatcggggaccgctacttcctgaaggccatcgaccagtactggcacgaggactgcctgagctgcgacctctgtggctgccggctgggtgaggtggggcggcgcctctactacaaactgggccggaagctctgccggagagactatctcaggctttttgggcaagacggtctctgcgcatcctgtgacaagcggattcgtgcctatgagatgacaatgcgggtgaaagacaaagtgtatcacctggaatgtttcaagtgcgccgcctgtcagaagcatttctgtgtaggtgacagatacctcctcatcaactctgacatagtgtgcgaacaggacatctacgagtggactaagatcaatgggatgatataggcccgagtccccgggcatctttggggaggtgttcactgaagacgccgtctccatggcatcttcgtcttcactcttaggcactttgggggtttgagggtggggtaagggatttcttaggggatggtagacctttattgggtatcaagacatagcatccaagtggcataattcaggggctgacacttcaaggtgacagaaggaccagcccttgagggagaacttatggccacagcccatccatagtaactgacatgattagcagaagaaaggaacatttaggggcaagcaggcgctgtgctatcatgatggaatttcatatctacagatagagagttgttgtgtacagacttgttgtgactttgacgcttgcgaactagagatgtgcaattgatttcttttcttcctggctttttaactcccctgtttcaatcactgtcctccacacaagggaaggacagaaaggagagtggccattctttttttcttggcccccttcccaaggccttaagctttggacccaagggaaaactgcatggagacgcatttcggttgagaatggaaaccacaacttttaaccaaacaattatttaaagcaatgctgatgaatcactgtttttagacaccttcattttgaggggaggagttccacagattgtttctatacaaatataaatcttaaaaagttgttcaactattttattatcctagattatatcaaagtatttgtcgtgtgtagaaaaaaaaaacagctctgcaggcttaataaaaatgacagactgaaaaaaaaaaaa

SEQ ID NO: 2 - DNA, 7232 bp, Human - Parkinson′s 41778
  misc_feature (5323)..(5323): indeterminate
tgttggcacaagattacaacctacaatttcaatgcccttccctcctacacagctcaaatggaaggaaactgattaacacatagaaaggaacggcaggctcacaggtttaagcatttgtttttacaaaaaggagtgggatatgggggtggtcaagttttacgggtaataacaacattctctctagatggcagggggtaagtctgcctgtcctccattgctggtaccacataagggatacattagtaaagtaaggtaagggttatatgtgtggctggcctgttctttagagaggaaggtgggatggtatctgctgtgtacctgtctcagaccaaacctggggctggaccagtgcccctttcttctcagccctcctccacagcctgactgtactgccagccgcacccatgagaaggaagtgttgaaggaagcctgaagacatccacctcgagaaaaggaaacaggaaaactattccttccgccttctctggaccaaattcactccctcagctccagagagtgctgcaaaaatcttccttagggacgctgtgaagacacggggaagaggccagccctgagcgggcaagaagaaagcttgaactttccatgcagtaccaagcaaggctattttggaaacaagcaaacaaacaaaaaatggaaaccaacaccatataaccacaggttgggtagctcctaaatcctggacagcatttggattgggggtaagggcaattttactccagctccttgtyttataacccgcacaaggtgccrrgaggttaaggaccacaactgacctctctttccctacaagcaccattccttggattcccagttttgttgagctcatttgaggtcatccttgccatcctcttaaaaaatgacacagcctcttggttattccattttctatccctttctayaattatgaacrcactctctaaacaggagatctatatatgctctcatcctgcaacccaagagattaggygacctaatgatgagaaggctaacttatttgctttcctgggtggtcctgatggaaatgaacagcagcagagttctggtgggatagagcagtctagtctgagccactgtgtgattccccaggggactggtccctgggtacatgggtccctggctctctgtctcagttccacagatattactcaacttggccatttacgcctcagctaaaacatatgagcatgagggaaaagggacaagaaatacttttgtcctctagaaaaacttaactcaatacatcttggctactttgcctcaacccacggggttccctggaaacatttgttgaacttggccaagaggagattgtatatggatggcacacctggaaaaggggagaaagcacactagttcatctcttttataagtagaatggtcccagggatgttaacctttctgatgctgatagtagatgagtggagtggggtaatcgtgaaaacttaaaaacccttaagctgtttggagactacwcaccacagtggagcaaaccaaggtggggcctcactgccctcctcagagcccatccctgaccatgtcattggataggatggaagacaaagtctytgaaatggaggctgggctggcaaaagtaagtatgaagatgctactactggccttccacttcctctctctgatacacctaagaaggtgagagacactagcttcaattccattttacaacagttctcaacaacctgaaatattcttgacttccttaagcacatggaaaactggtaagctatatctttctaactgaaattttacttattctaaccaaccttttctttgattatcaaccagtttgacagtcttcagatcacaaaacaatagaacagacctgacatttacaaaactgtactacatacttatttcttttacagtagagctgaccaattacttttctaactactaatgcaacaccatgccactgggccctggactctgatgtttctgatccctgagcaacacatctatgtccattcacagatggaaatgaaagagtgagattctcagaggctaaacaacattctgggaagcaaattggatttttgaagggagagtcctccttagaaaattctggagccaaacatgctcccagtcacgagggaatggkktragccgggccaggtttcctgtctgtgrgctatagctcccttcctgaactccctcmtgtyctttgggaaggacacaagcytcttgctcattaagcaaaggccctccaagtctcctttttacttcagcactttggaaacyytccaagacttaacttccgatctggggaggttctacgtggagcagacctatcccttcttattcctgttcctgggtgtctccataatcagcctctaccttccctggcataagtcatatctgttatgctgcaggaacygagtaaccaaagctagatatcccttggaccactgattagtgacacattttctccacaacaaagggaggttaaagacaaaggtttaaccctgagaaactttttttttggcaagataataaagaggttaaagaggctacgtggatctgtcactgctttagcttaccattttcaattcagcaccaaagtgctcagaaagacaggctcagwtcaggacgacttcttaaagggatacacaggcaatattactggcaaaatgattttccaaagctgagccacgcgccccaccctacagggaggggagtagaatcttatggcccagcattggcattgaggcatcttgctttttttcctcagaattccacaaaggcttaagagtgatctaaatgcctggctggaagctcaagtctaggagagctattttactgcactgttgagatgttctctgcaaggtagtcacagataataagcaatttagtagataattattctatacaagaattaatgttctctgaaacagtgattaatagaatcttcacatctggcctggctgggaggcttgggggcaggaaagggagggtggaaggatgggtagcacatcaaaatggtgatgaactggcaaaacgacaatgctgctgtttttgtatcctttaaagcctatatcccagggttttaaacgacgccgggggtgtgcttcacatctggcctggctgggaagcttgggggcaggaaagggaaggtggaaggatgggtagcacatcaaaatggtgatgaacctggcaaaacgacaatgctgctgttttgtatccctttaaagcctataacagtttaatgacgcttttttkgtgtgcttcctgataggaagtcaaataagttttagtattaagaactggggatataamccaaaggaaacctagagtcaactgactgctggaggaacagaagcaatgrctttctttccacccttcttagatagtaatcttacttttgttggctaaaagcactttcttcagcattctgagttaacacgggttccctataagctcccctccccaggtggacagtcytattttctaagacaagctcaaagctcaaactcaccaaattaccactataaacaaagcttcagggaagagacataacctwacttcaaaagcttcaggtcagagacrgccaaaatcatgtgactgctagcytcarstcaagyagtgtgcccaaatcattcacaaaatgggattccctttccttttcttttttttttttttggagacggagtcttgtctgtcgctaggctgcagtgcagtggcgggatctcggctcactgcaacatccacctcctgggttcaagcgattttcctgcttcagcttcccgagtagctgggactacaggagcgtgccahtacacctggctaatttttgtatttttagtagagatggggtttcaccatattggtcaggctggtcttgaactcctgaccttgtgatccacccgcttcggcttcccaaagtgctgggattacaggcgtgagccaccatgaccagccctccatttattaatttataaggggactagaaggacaaactatttgattaatttgaaggtgagaggtatcattttctcagatgctccaactaggcttctcacatccctcaaaggttatcagctaaagacaagagtttctgaggctaatgcaaaaacatggtcaaatgtgaaacksccytttwrgaggcagggtttgtgaaaggctgctggcytccaaaggagagtatgcmtaaactctctggrctcccaggacctgtggtgacctcaagctaacctgatgcacagacaacagtatcttttagggactgcagccaatgttcccccaatttacttttttttttataccttaaaatatcaaatgtttcatttttaggtgatatcccaagtgacacagaaacacagctcctccggatggtgaagtctccgctgcaaaactcacgaaacagcacagaggatgactctagggtgctctcccacttgctccaagttaggcaggtgccctgatggaggcaaggctgagccattaacatgcacagctccatatcctggaagcctctgtagccttgaggagataggctctatccaacaggactcagaataaatgactgccacctcaactgagcagtatgaactgcccacctcaactrggcagtctgacctcttccacagtctttaagaaacagaagagccatttgcattatttaatcaccactcaataatctcctccctccccatagctgcttgagttaactgtcccacaagtgtcctgtgtttwgtawagatgagggttattttctgawatgcagatgggamtaggatgaagctgtattatcaattacagagacagccaactctcatttctcttaaagcaaaaattcattccactgtgatttctccaggtatttcctgcaattctaaatccgcccttgatattagagaatattaaaatcacacagttgtggcaaaaatcacattgtggctattaaatcctttttgtgaactaaaaaaagtgaatgtggcttaggctaagctgttttttttttttttttttttaaagatgaggatgcggactccaacaaaggcattaagaaagtactagatgaaaatgagaaatatgtgaaggataacatgtgaaatgtacactcaggtctaacaaatacctattatttctctggttaagaaggtttagcaggagcctccaatgagcactgtatgtacagaaaagggaaggagcaggaggaggaacagatctgcacagaatttttttcttaaaaaccacaaagggtgacttttttcttctaagcaagcaagcctgagaggcattacatgggctggctcctaatatcaaaacaaaatatttctttgccacnaaaggaacttgactatgtagcaacacatttacnaaaactactgcaaaacactcccagagggcagtgacctactctngbtccccagaggcttccaagaagcaaggcctncaagtgccytcatgttccatgggggctgtgcagtgcaatgcagaaggcggaaatcctgctgtgacgccaccctcgagaccttctgcagcctgagagggggtgatactcccacaccctttgatccttccagatggctcagcctagatctagcagtgagagagccctctccatgcagggcagatggagtggtggacacttgtgaaaacaaaacactaactgttccatcctgttatatttgctgtgaggaaaattaagattcctgttgtatgggctgcactgtttctggaagactacagaaaatctaacatggttgacacttcctggtagcccttctgtacatacacacacacaacccagagagargacagagagaaaatcctggtccaaargatcacatgaccttactagtgtttccccaatgactgtaatttataaactaaaaatttttacaaatccactgctatcttcttctgtcctgagtttggtagactttaatggatgctccagcaataaccagaatctaggacatgcagactcactgtgagcgagaggctagggatctgccctaaacataggaacctgtttctatcaagcctgaatgaggtcagctctggtagaattaatgacaaatcaatgtcagtgaaatattctgcaaacagggtagcttttgtgctttcttttgattattttctttggggagataaaggtattgcaaccatgggtctaactaatctatcactaaaggactgtgacgacattcctcagtaaagacagtcatggtgtttactgtgccmargraggraaaatcccagrtcatcagaatcccagtgcaacagaaaacagcaccagtttcaacaattctgatgttggacaaagcctctttttgtcagttaagaaagcgtaagcacaatctctcagtcaattgcattaacaggatccattcatgtcactgaaaratacttcttggcacagttcactttccgtgacccaaagacaccaacaacaatcaaaactgcactttcagggtagtatattgttacactagcccagtcaaaacatactcagaacatttaaaaatgtataaattactgttagcactgcagttccgttttattgtcttatttatttattgagacagagtcttactctgttgcccaggctggagtgcagtggcatgatcttggcttactgcaacttctgcctctcaggtttaagcaattctcatgcttcagcctaccaagtagctgggattacaggagtgcaccatcatgcccagctartttttgtattttagtagagatgggsttttrccaygttggtcaggctggtctcaaactcytggcctyaagtgacccrcctgccttggcytcccaaagtgstgggattacaggtgtgagccaytacacccagccmtgcagtccctttttaaagagtactgactgwytycctgcamagtactttcaaatccayaccataaaggtagaatttatctggaagaatgtgttttttgaattccacctggcacttggttaaatccactttattttgccctttatttatttgaacagaactttagaatgctctaggtcaggaaaagaaacagtgtgttcgtttttaagaaggatgtgtgaatagtaagttggcattcagactcaagtcccatggtattgttcttatccaatgggacctctccttgggaatataattcctttaccagaggtatcatggtatcccaagaatataactgctcccttggctctgtgtgtgaagattgggggagaggatgtaaactaagagtata

(19) United States
(12) Patent Application Publication
(10) Pub. No.: US 2003/0104393 A1
(43) Pub. Date: Jun. 5, 2003

(54) Blood assessment of injury

(76) Inventors: Frank R. Sharp, Cincinnati, OH (US); Yang Tang, Cincinnati, OH (US); Aigang Lu, Cincinnati, OH (US)

Correspondence Address: DINSMORE & SHOHL, LLP, 1900 CHEMED CENTER, 255 EAST FIFTH STREET, CINCINNATI, OH, 45202, US

(21) Appl. No.: 09/996,275

(22) Filed: Nov. 28, 2001

Related U.S. Application Data
(60) Provisional application No. 60/253,568, filed on Nov. 28, 2000.

(51) Int. Cl. C12Q 1/68

(52) U.S. Cl. 435/6

(57) ABSTRACT

Methods of injury assessment in an individual include the steps of determining a pattern of expression exhibited by blood cells obtained from an individual and comparing the pattern of expression exhibited by the obtained blood cells to an injury database to assess the injury.

### RELATED APPLICATION

This application claims priority under 35 U.S.C. §119 of U.S. Provisional Application Serial No. 60/253,568 filed Nov. 28, 2000.

### FIELD OF THE INVENTION

The present invention is directed toward methods of assessing injury in an individual, wherein injury is defined as cell death, cell dysfunction, or genetic abnormalities either acquired or inherent, any of which are present in an occult, acute or chronic stage. More particularly, the invention is directed toward methods of injury assessment which comprise determining a pattern of expression exhibited by obtained blood cells and comparing the pattern of expression exhibited by the obtained blood cells to an injury database to assess the injury.

### BACKGROUND OF THE INVENTION

Non-invasive diagnostic methods such as computed tomography (CT) and magnetic resonance imaging (MRI) are useful in diagnosing injury resulting from ischemia, tumors, bleeding, trauma, toxins, infection, autoimmune disease and other etiologies. Invasive imaging methods include positron emission tomography (PET) and single photon emission computed tomography (SPECT), which require the injection of radioisotopes, and cerebral angiography and myelography, which require the injection of radiopaque dyes. A further invasive procedure for assessing injury is through the use of a biopsy. many factors, including cardiac arrest, strokes, hemorrhages, hypoglycemia episodes, head injuries, seizures, psychiatric diseases, infection, toxins, drugs, as well as coma due to liver, renal, endocrine or pulmonary failure. Such patients may be unable to respond to requests regarding a medical history or conditions. Further, it is often difficult to transport or to use imaging technology on artificially ventilated patients in intensive care units or post-surgical units. Still further, it is complicated to perform a biopsy when the source or the cause of the injury may be unknown. Thus, it would be useful to have a convenient method of assessing injuries that does not require a biopsy, imaging or transfer of the patient, and can be done with procedures no more invasive than the withdrawal of a blood sample.

Neither CT nor MRI are useful for diagnosing injury where there is isolated dysfunction or isolated loss of neurons or individual cells in the blood, brain, spinal cord, lung, muscles, nerves or other organs. For example, there are no convenient methods for determining whether injury to cells in the brain, blood, muscle, nerves, heart, lung, endocrine glands or other organs has occurred following hypoglycemia, hypoxia, drug over-dose, coma, status epilepticus, stroke, or severe hypotension due to cardiac arrest or other causes. In addition, even with these imaging methods there are numerous injuries that cannot be conveniently or adequately assessed. For example, patients suffering cardiac arrest with cardiovascular collapse often have diffuse neuronal injury in the brain and in other organs that cannot be visualized. Similarly, injury caused by hypoxia, hypoglycemia, or status epilepticus cannot be diagnosed with such methods. Thus, it would be useful to have a convenient and adequate method to assess injury states.

Many individuals remain asymptomatic for an injury for numerous years. Such individuals do not seek medical treatment because the injury is not prevalent. In addition, such individuals cannot report an accurate medical history because they are not aware of a hidden medical condition. Therefore, it is nearly impossible to accurately assess injury in these individuals when symptoms are not overtly expressed. Thus, it would be useful to have a convenient method of assessing asymptomatic injuries to continuously monitor an individual's health.

The prior art teaches that specific genes or proteins have been identified that correspond with a particular specific disease. In addition, these genes and proteins can be classified using microarray technology. The identification and measurement of these specific genes and proteins allow a specific disease to be diagnosed.

For Example, Barone, et al., J. Cereb. Blood Flow Metab., 19(8):819-834 (1999), teach that transforming growth factor (TGF), tissue necrosis factor (TNF), interleukin-1 (IL-1), interleukin-8 (IL-8), heat shock proteins, and metalloproteinases may be induced, for example, in the brain during a stroke. Bergeron et al., European Journal of Neuroscience, 11:4159-4170 (1999), teach that hypoxia-inducible factor-1 (HIF-1), glucose transporter-1 (GLUT-1), and several glycolytic enzymes are upregulated in, for example, the brain during focal ischemia. HIF-1 is induced by hypoxia, but not by hypoglycemia—making this gene a candidate for distinguishing between hypoxia and hypoglycemia in blood, the brain and other organs. Sharp et al., TINS, 22:97-99 (1999), teach that heat shock proteins (HSPs) and glucose-regulated proteins (GRPs) are produced in response to ischemia and other stresses. HSPs are induced in response to denatured proteins, GRPs are induced in response to low glucose, and ORPs (oxygen regulated proteins) are induced in response to low oxygen. Martens et al., Stroke, 29:2363-2366 (1998), teach that S-100 protein, a calcium-binding protein, may be a serum marker of brain damage useful for clinical assessment. Martens et al. further teach that cardiac arrest may produce cerebral damage that can be detected by release of neuron-specific enolase to the cerebrospinal fluid and eventually to the blood.

Microarrays of DNA have been used to classify types of cancer, as taught by Alizadeh et al., Nature, 403:503-511 (2000), and Golub et al., Science, 283:531-537 (1999). Microarrays have also been used in analyzing inflammatory diseases such as rheumatoid arthritis and inflammatory bowel disease, as taught by Heller et al., Proc. Natl. Acad. Sci., U.S.A., 94:2150-2155 (1997). Friend et al, (Rosetta Inpharmactics, Inc.) U.S. Pat. No. 6,218,122 (2001), teach a method for monitoring disease states and levels of effect of therapies using gene expression profiles derived from cellular constituents indicating aspects of the biological state of the cell, such as RNA or protein abundances or activity levels. Erlander et al (Ortho-McNeil Pharmaceutical, Inc.) WO 00/28092 (2000), teach a method for the production of gene expression profiles from a selected set of cells residing in a given tissue/organ. Friend et al, (Rosetta Inpharmactics, Inc.) WO 00/24936 (2000), teach methods of using co-regulated genesets to enhance the detection and classification of specific gene expression patterns for a specific biological state. Ralph et al., (Urocor, Inc.) U.S. Pat. No. 6,190,857 (2001), teach that a specific human disease state may be detected in circulating leukocytes by identifying specific genomic markers for the specific disease state.

However, even with the progression in the art, there remains a substantial need for convenient and adequate methods that can assess an injury for an individual. It would also be advantageous to provide methods of assessment which could be conveniently and adequately used in particular individuals who are asymptomatic, artificially ventilated and/or in altered states of consciousness, and that go beyond current methods of clinical diagnosis.

There is also a substantial need for methods of assessment that could utilize a relatively non-invasive procedure for diagnosis, prognosis, and/or monitoring an injury state.

### SUMMARY OF THE INVENTION

Accordingly, it is an object of this invention to provide convenient methods of assessing injury.

In accordance with one aspect of the invention, there are provided methods of injury assessment in an individual. The methods comprise the steps of determining a pattern of expression exhibited by blood cells obtained from the individual and comparing the pattern of expression exhibited by the blood cells to an injury database to assess the injury. In specific embodiments, the pattern of expression may be a pattern of gene expression, protein expression, or combinations thereof, and the injury database may be a genomic database, proteomic database, or combinations thereof. Furthermore, the injury database may be based on a specific organ or a specific injury cause or disease.

In accordance with another aspect of the invention, there are provided methods of stroke injury assessment of an individual comprising the steps of obtaining a peripheral blood sample from the individual, capturing a pattern of expression, defining a pattern of expression, and comparing the pattern of expression exhibited by the blood cells to an injury database to assess stroke injury.

In accordance with yet another aspect of the invention, there are provided methods of hypoxia injury assessment of an individual comprising the steps of obtaining a peripheral blood sample from the individual, capturing a pattern of expression, defining a pattern of expression, and comparing the pattern of expression exhibited by the blood cells to an injury databases to assess hypoxia injury.

In accordance with a further aspect of the invention, there are provided methods of hypoglycemia injury assessment of an individual comprising the steps of obtaining a peripheral blood sample from the individual, capturing a pattern of expression, defining a pattern of expression, and comparing the pattern of expression exhibited by the blood cells to an injury bank to assess hypoglycemia injury.

In accordance with yet another aspect of the invention, there are provided methods of seizure injury assessment of an individual comprising the steps of obtaining a peripheral blood sample from the individual, capturing a pattern of expression, defining a pattern of expression, and comparing the pattern of expression exhibited by the blood cells to an injury database to assess seizure injury.

In accordance with yet another aspect of the invention, there are provided methods of movement disorder injury assessment of an individual comprising the steps of obtaining a peripheral blood sample from the individual, capturing a pattern of expression, defining a pattern of expression, and comparing the pattern of expression exhibited by the blood cells to an injury database to assess movement disorder injury.

In accordance with yet another aspect of the invention, there are provided methods of diabetes injury assessment of an individual comprising the steps of obtaining a peripheral blood sample from the individual, capturing a pattern of expression, defining a pattern of expression, and comparing the pattern of expression exhibited by the blood cells to an injury database to assess diabetes injury.

In accordance with yet another aspect of the invention, there are provided methods of infectious disease assessment of an individual comprising the steps of obtaining a peripheral blood sample from the individual, capturing a pattern of expression, defining a pattern of expression, and comparing the pattern of expression exhibited by the blood cells to an injury database to assess infectious disease injury.

In accordance with yet another aspect of the invention, there are provided methods of immune mediated disease assessment of an individual comprising the steps of obtaining a peripheral blood sample from the individual, capturing a pattern of expression, defining a pattern of expression, and comparing the pattern of expression exhibited by the blood cells to an injury database to assess immune mediated disease injury.

In accordance with yet another aspect of the invention, there are provided methods of efficacy or toxicity assessment, or combinations thereof, of an individual comprising the steps of obtaining a peripheral blood sample from the individual, capturing a pattern of expression, defining a pattern of expression, and comparing the pattern of expression exhibited by the blood cells to an injury database to assess efficacy or toxicity, or combinations thereof. The methods can be used, for example, for assessing efficacy and/or toxicity of drugs or environmental toxins.

In accordance with yet another aspect of the invention, there are provided methods of psychosis assessment, or combinations thereof, of an individual comprising the steps of obtaining a peripheral blood sample from the individual, capturing a pattern of expression, defining a pattern of expression, and comparing the pattern of expression exhibited by the blood cells to an injury database to assess psychosis.

In accordance with yet another aspect of the invention, there are provided methods of headache assessment, or combinations thereof, of an individual comprising the steps of obtaining a peripheral blood sample from the individual, capturing a pattern of expression, defining a pattern of expression, and comparing the pattern of expression exhibited by the blood cells to an injury database to assess headache.

In accordance with yet another aspect of the invention, there are provided methods of genetic disorder assessment, or combinations thereof, of an individual comprising the steps of obtaining a peripheral blood sample from the individual, capturing a pattern of expression, defining a pattern of expression, and comparing the pattern of expression exhibited by the blood cells to an injury database to assess the genetic disorder.

In accordance with yet another aspect of the invention, there are provided methods of proliferative disease assessment, or combinations thereof, of an individual comprising the steps of obtaining a peripheral blood sample from the individual, capturing a pattern of expression, defining a pattern of expression, and comparing the pattern of expression exhibited by the blood cells to an injury database to assess the proliferative disease disorder.

The present methods are advantageous in providing convenient, relatively non-invasive diagnosis of injury in occult, acute or chronic stages. Additional embodiments, objects and advantages of the invention will become more fully apparent in view of the following description.

### BRIEF DESCRIPTION OF THE DRAWINGS

The following detailed description will be more fully understood in view of the drawings in which:

FIG. 1a is a Venn diagram showing the numbers of genes that were upregulated more than twofold in blood 24 hours after brain ischemia (BI), brain hemorrhage (BH), and sham surgery (S), compared with untouched control individuals, as described in Example 2;

FIG. 1b is a Venn diagram showing the numbers of genes that were downregulated more than twofold in blood 24 hours after kainate (K), insulin-glucose (IG), and hypoxia (H), compared with untouched control individuals, as described in Example 2;

FIG. 2 is a cluster analysis of the pattern of expression obtained from individuals with kainate, insulin-glucose, hypoxia, brain ischemia, brain hemorrhage, as compared to sham surgery and untouched control individuals, as described in the Example 2;

FIG. 3a is a graph which demonstrates the identification of Dead Box Y Isoform, which is differentially expressed in two groups of patients, males and females, as described in Example 3;

FIG. 3b is a graph which demonstrates the identification of Ribosomal Protein S4 Y Isoform, which is differentially expressed in two groups of patients, males and females, as described in Example 3;

FIG. 4 is a graph which demonstrates that genes SEQ ID NO:1 and SEQ ID NO:2 are expressed more highly in Parkinson's individuals as compared to other individuals without Parkinson's, as described in Example 4;

FIG. 5 is a cluster analysis of the expression obtained from pediatric epilepsy patients prior to being treated compared to the expression of these individuals after being treated with anticonvulsant valporate (VPA) or the anticonvulsant carbamazepine (CPZ), as described in the Example 8;

FIG. 6 is a cluster analysis of the pattern of expression obtained from individuals with neurofibromatosis, as described in Example 9;

FIG. 7 is a cluster analysis of the pattern of expression obtained from individuals with bipolar, as described in Example 10;

FIG. 8 is a cluster analysis of the pattern of expression obtained from individuals with acute migraine headaches, as described in Example 11;

FIG. 9 is a cluster analysis of the pattern of expression obtained from individuals with schizophrenia, as described in the Example 12; and

FIG. 10 is a cluster analysis of the pattern of expression obtained from individuals with Tourettes, as described in the Example 13.

### DETAILED DESCRIPTION

Upon injury, the blood, in particular the blood cells, will be exposed to environmental stresses, immune responses or additional effects associated with the injury. The inventors have found that blood cell responses can be used to determine whether there has been injury to neurons or injury to other cells in the body, the cause of the injury, and/or the degree of the injury. Methods in accordance with the invention may be used to detect remote injury. In addition, methods in accordance with the invention may be used to assess injury that cannot be conveniently or adequately evaluated by current blood tests, by imaging or biopsy, and may conveniently be used on all individuals, including individuals who are asymptomatic, in altered states of consciousness, and/or who are artificially ventilated. Advantageously, methods in accordance with the present invention are relatively non-invasive and do not require biopsy or the injection of radioisotopes or radiopaque dyes.

As used herein, “assessment” is intended to refer to the prognosis, diagnosis, or monitoring of an injury based upon a pattern of expression from a blood sample. As used herein, “individual”, is intended to refer to an animal, including but not limited to humans, mammals, and rodents. As used herein “blood cells”, is intended to refer to nucleated cells of the blood, including but not limited to red blood cells, white blood cells, lymphocytes, leukocytes, monocytes, macrophages, eosinophils, basophils, polymorphonucleic cells, all other subsets of cells containing RNA or protein, or combinations thereof.

As used herein, “injury” is intended to refer to genetic abnormalities, either inherent or acquired; death of cells; or dysfunction of cells produced by a wide variety of overt or covert states including, but not limited to, diffuse systemic disease, hyperproliferative cellular conditions, including benign, and non-benign or metastatic cancer, hemorrhage, infarction, ischemia, hypoxia, seizures, psychiatric illnesses, neurological diseases, hypoglycemia, trauma, toxins, drugs, organs, inflammatory diseases, autoimmune diseases, infectious diseases, demyelinating diseases, tumors, cancer, endocrine diseases, degenerative and metabolic diseases, including Alzheimer's, and infection, present in an occult, acute or chronic stage.

Autoimmune diseases include, but are not limited to, Graves, Rheumatoid arthritis, Thyroiditis/hypothyroidism, Vitiligo, IDDM, Multiple sclerosis, Primary glomerulonephritis, Systemic lupus erythematosus, Sjogren's, Addison's disease, autoimmune hemolytic anemia, chronic active hepatitis, Goodpasture's syndrome, idiopathic thrombocytopenia purpura, myasthenia gravis, myocarditis, pemphigus, pernicious anemia, polymyositis, primary biliary cirrhosis, relapsing polychondritis, rheumatic fever, scleroderma, and uveitis. Psychiatric illnesses include, but are not limited to, schizophrenia, generalized anxiety, panic disorders, post traumatic stress, obsessive compulsive, phobias, social anxiety disorder, major depressive disorder, bipolar, alcohol and drug abuse, and eating disorders.

As used herein, “organ injury” is meant to refer to injury to one or more organs, including but not limited to, the following: brain, organs of the special senses including eyes, ears and nose, the central nervous system, the spinal cord, nerves, muscles, heart, lung, kidney, liver, genitalia, endocrine glands, bladder, gastrointestinal system, joints, bones, blood vessels, and blood cells, including red blood cells and white blood cells, and including lymphocytes, leukocytes, monocytes, macrophages, eosinophils, basophils, and all other cells found in blood.

As used herein, “glucose-inducible genes” is intended to refer to genes which are induced by changes in serum or blood glucose levels, usually low glucose levels, and decreased with high glucose levels; while “glucose-related proteins” is intended to refer to gene products which are produced or which levels are varied in response to changes in serum or blood glucose levels, preferably low glucose levels. “Low glucose levels” is intended to refer to glucose levels below the range generally regarded by physicians as normal. As used herein, “hypoxia-induced factors” is intended to refer to factors which are produced or which levels are varied in response to hypoxia.

As used herein, a “genomic injury bank” refers to a library composed of DNA, RNA, or combinations thereof, isolated from blood samples. As used herein, a “proteomic injury bank” refers to a library composed of protein isolated from blood samples. As used herein, an “injury database” refers to a database comprising a pattern of expression or patterns of expressions indicative of a single or different states of injury, including but not limited to pattern of gene expression, protein expression, or combinations thereof. The injury database may be based on a specific organ or a specific injury cause or disease. Organ specific injury databases include, but are not limited to, brain injury database, spinal cord injury database, blood injury database, muscle injury database, nerve injury database, lung injury database, liver injury database, heart injury database, kidney injury database, genitalia injury database, eye injury database, ear injury database, nose injury database, teeth injury database, bone injury database, white blood cell injury database, endocrine gland injury database, gastrointestinal injury database, blood vessel injury database, or combinations thereof. Cause/disease specific injury databases include, but are not limited to, global ischemic injury database, focal ischemic profile, status epilepticus injury database, hypoxia injury database, hypoglycemia injury database, cerebral hemorrhage injury database, hemorrhage injury database for one or more organs, diabetes complications injury database, psychosis injury database, psychiatric disease injury database, bipolar injury database, schizophrenia injury database, headache injury database, acute migraine headache, database, endocrine disease injury database, uremia injury database, injury database for ammonemia with hepatic failure, toxin overdose injury database, drug overdose injury database, Alzheimer's disease injury database, Parkinson's disease injury database, Tourettes disease injury database, muscle disease injury database, proliferative disease injury database, neurofibromatosis injury database, nerve disease injury database, other dementing illness injury database, inflammatory diseases injury database, autoimmune diseases injury database, infectious diseases injury database, demyelinating diseases injury database, trauma injury database, tumors injury database, cancer injury database, degenerative and metabolic diseases including Alzheimer's injury database, genetic or familial diseases injury database, or combinations thereof.

As used herein “stroke” or “cerebrovascular accident” is intended to refer to cerebral infarction resulting from lack of blood flow and insufficient oxygen to the brain. As used herein, “infarction” is intended to refer to tissue/cell death. In an ischemic stroke, the blood supply is cut off due to a blockage in a blood vessel, while in a hemorrhagic stroke the blood supply is cut off due to the bursting of a blood vessel.

As used herein, “pattern of expression” is meant to refer to the representation of molecules, including but not limited to genes, proteins or combinations thereof, in an injury state, which are upregulated, downregulated or embody no change. As used herein, “expression method” is meant to refer to any method known in the art that can define a pattern of expression, such as the significance analysis of microarrays and class prediction, as taught by Tusher, Proceedings National Academy of Sciences, 98: 5116 (2001). These methods may assess injury at a point minutes, hours, days or weeks after the injury has occurred, owing to rapid and/or prolonged expression of the molecules indicating the injury.

Patterns of expression may be derived from, but are not limited to, the following detailed injuries. For example, in individuals who sustain a brief period of severe hypoglycemia (low serum glucose) because of oral or injected hypoglycemics or because of severe illnesses there may be an induction of glucose-inducible genes in all of the blood cells, including polymorphonuclear cells (neutrophils), lymphocytes and macrophages. Hypoglycemia may also damage brain cells, blood cells, cells in the pancreas, cells in the heart, lung and other organs. Thus, gene and protein expression in the blood cells may change in response to the hypoglycemia.

In individuals who sustain a period of pure hypoxia during anesthesia or while on a respirator there may be an induction of a set of genes specific for hypoxia; therefore, glucose-inducible genes may not be induced. In contrast, in individuals sustaining a cardiac arrest, wherein the brain, other organs and blood become ischemic for a length of time, there may be an induction of genes regulated by low glucose and low oxygen, as well as genes that are related to acidosis and ischemia. Thus, the genomic and/or proteomic response which may be observed in blood cells during episodes of pure hypoxia may differ from those observed in blood cells during episodes of pure hypoglycemia.

An individual having status epilepticus has brain injury manifested by isolated neuronal injury. The removal of such dead neurons is performed by monocytes and macrophages. Thus, during status epilepticus there may be selective change in genomic and/or proteomic expression of macrophages. Further, during repeated seizures there may be little white cell hypoxia or hypoglycemia, thus, hypoxia-induced factors, glucose-related proteins and heat shock proteins will not be induced. Additionally, during prolonged seizures there may be massive sympathetic discharge. The individuals may have elevation of catecholamines (e.g., epinephrine) that may stimulate adrenergic receptors in the blood cells.

If a individual is suffering from one or several focal strokes, blood cells respond to the site of the injury, the brain, and the response is targeted to brain antigens with removal and repair of neurons, glia, and vessels. During severe ischemic hypotension and infarction of the brain or other organs, hypoxia-induced factors, glucose-related proteins, and heat shock proteins are all induced. In heavy metal toxicity, heat shock proteins may be induced.

It has been found that molecules regulate in accordance with an injury state to determine a pattern of expression. In an embodiment of the invention, the number of molecules necessary to define a pattern of expression is at lease about 10. In an embodiment of the invention, the number of molecules necessary to define a pattern of expression is at lease about 50. In a further embodiment of the invention, the number of molecules necessary to define a pattern of expression is at least about 200. In a further embodiment of the invention, the number of molecules necessary to define a pattern of expression is at least about 500. In a further embodiment of the invention, the number of molecules necessary to define a pattern of expression is at least about 1000. In a further embodiment of the invention, the number of molecules necessary to define a pattern of expression is at least about 5000. In a further embodiment of the invention, the number of molecules necessary to define a pattern of expression is about at least 10,000. In a further embodiment of the invention, the number of molecules necessary to define a pattern of expression is about at least 50,000. In a further embodiment of the invention, the number of molecules necessary to define a pattern of expression is about at least 100,000. In a further embodiment of the invention, the number of molecules necessary to define a pattern of expression is all molecules represented in the injury state. The upper and/or lower limit of molecules necessary to define a pattern of expression may similarly vary in individuals applications of the present method, and in specific embodiments may be 10, 50, 200, 500, 1000, 5000, 10,000, 100,000, or the like.

In accordance with another embodiment of the invention, the molecules, which may be used in determining a pattern of expression by blood cells include, but are not limited to, intermediate metabolism, immune-related molecules, cytokines, chemokines, immediate early genes, structural genes, neurotransmitters, receptors, signaling molecules, oncogenes and proto-oncogenes, heat shock and stress genes, transporters, trophic and growth factors, cell cycle genes, lipid metabolism, arachidonic acid metabolism, free radicals and free radical scavengers, metal binding, transporting genes, or combinations thereof.

In accordance with yet another embodiment of the invention, various enzymes whose expression may be evaluated comprise aldolase-A, lactase, dehydrogenase-A, phosphofructokinase-L, pyruvate kinase-M, hypoxia-inducible factor, or combinations thereof, while heat shock proteins whose gene expression may be evaluated comprise ubiquitin, HSP10, HSP27, HSP25, HSP32 (also known as heme oxygenase-1 or HO-1), HSP47, HSP60, HSC70 (also known as HSC73), HSP70 (also known as HSP72), HS90, HS100/105, or combinations thereof.

In accordance with a further embodiment of the invention, the classes of genes and proteins further comprise intermediate-early genes (IEGs), the genes for hypoxia-inducible factor 1 (HIF-1), glucose transporter-1 (GLUT-1), glycolytic enzymes, transforming growth factor (TGF), tissue necrosis factor (TNF), interleukin-1 (IL-1), interleukin-1 receptor antagonist (IL-1 RA), interleukin-8 (IL-8), heat shock proteins (HSPs), glucose-regulated proteins (GRPs), oxygen-regulated proteins, metalloproteinases, nitric oxide synthase (NOS), cyclooxygenases (COX), poly(ADP-ribose) polymerase (PARP), calcium-binding proteins such as S-100 proteins, histamine H2-receptor, c-jun leucine zipper interactive protein, Glut3, the vesicular monoamine transporter, TNF intracellular domain interacting protein, vascular tyrosine phosphatase, glucose-induced genes, hypoxia-induced genes, transcription factors, signaling factors, growth factors, transmitters, receptors, membrane protein genes, peptides, cytokines, chemokines, structural genes, cell cycle genes, apoptosis-related genes, acidosis-induced genes, ischemia-induced genes, enzymes, kinases, phosphatases, trophic factors, nuclear factors, hormones, or combinations thereof. Hypoxia-induced genes comprise genes for heat shock proteins, genes for nitric oxide synthase, genes for matrix metalloproteinases, genes for cyclooxygenases, genes for growth factors, genes for hypoxia-induced factors such as HIF-1, and genes involved in the production of cytokines, chemokines, adhesion molecules, or combinations thereof. Glucose-induced genes comprise glucose regulated proteins, glycolytic enzymes, glycosylated proteins, genes as listed in Table 3, or combinations thereof. Acidosis-induced genes comprise the genes as listed in Table 2, genes listed in Table 3, or combinations thereof. Ischemia-induced genes comprise the genes as listed in Table 3 or combinations thereof. Parkinson-related genes may comprise SEQ ID NO:1, SEQ ID NO:2, or combinations thereof.

The pattern of expression exhibited by the obtained blood cells may be captured by any method known to the art. An exemplary method is through the use of microarrays, for example using DNA microarrays, protein microarrays, peptide microarrays, or combinations thereof. Microarrays refer to surface microarrays, membrane microarrays, bead microarrays, solution microarrays, and the like comprised of nucleic acids, nucleic acid mimetics, discrete nucleotide sequences, preferably DNA or RNA sequences, discrete proteins, antibodies, protein fragments, antibody fragments, antibody-mimetics, peptides, peptide-mimetics, organic molecules and/or other molecules capable of selectively and specifically binding specific RNA, DNA or proteins; or subsets of RNA, DNA or protein molecules thus permitting the detection and measurement of the associated molecules for the purpose of capturing a pattern of expression.

In one embodiment of the invention, microarrays are used to capture the pattern of gene expression. The nucleotide sequences in two DNA samples or two RNA samples, such as, for example, the RNA isolated from two different cell populations, are compared by first labeling the samples, mixing the samples and hybridizing them to arrayed DNA spots. Generally each nucleotide sequence is labeled with a different flourescent dye or other labeling technique. As the samples are differentially labeled, it is possible to determine the pattern of gene expression.

To prepare RNA for use in a microarray assay, it is generally purified from total cellular content. Suitable methods of RNA isolation are known in the art and include the use of standard isolation methods, specific columns, or other collection methods. The RNA may be reversed transcribed to complementary DNA (cDNA) and in some applications to complementary RNA (cRNA). Either the labeled cDNA or the labeled cRNA may be used in the microarray assay.

Generally, the cDNA or cRNA samples are labeled, for example, with fluorescent dyes (fluors). Common fluors include Cy3 and Cy5. The labeled samples are referred to as probes. The probes are hybridized to a DNA sequence in the microarray. If the labeled probe contains a cDNA or cRNA whose sequence is complementary to the DNA at a given spot in the microarray, the labeled probe will hybridize to that spot, where it can be detected by its fluorescence. Since the probes are tagged with fluorescent molecules like Cy3 and Cy5 that emit detectable light when stimulated by a laser, the probes may be scanned and the emitted light recorded. The probe may be applied to a microarray, DNA, RNA or protein.

In a further embodiment of the invention, a microarray comprises from about 1,000 to about 100,000 DNA sequences. A sample is obtained from the patient's blood cells and is labeled with a first label, and a second RNA sample which serves as a control is labeled with a second label. The first label and the second label have different emission wavelengths. The labels may be fluors, biotinylated markers or other suitable markers. The labeled patient sample and the labeled control samples are mixed and hybridized to the microarray, or they are hybridized to separate arrays. Generally the microarray is then rinsed to remove any non-hybridized samples. The light emitted from the fluors may be measured using any method known in the art, such as commercially available scanners. The relative abundance of the patient and control samples hybridized to the various DNA sequences of the microarray are determined and a pattern is captured.

In yet another embodiment of the invention, the RNA is isolated from the blood of the hypoglycemia, hypoxia, status epilepticus, ischemic stroke, hemorrhagic stroke, and controls. The RNA is purified using standard methods, and then transcribed either into labeled cDNA or into labeled cRNA. These samples are then applied to custom microarrays that are fabricated using the methods for suppressive subtraction hybridization, or custom arrays made from commercially available cDNA libraries. The experimental samples are labeled with Cy3 and the untouched control or sham control samples are labeled with Cy5. The two samples are mixed and applied to a cDNA array produced from all available rat cDNAs, or from an array produced from cDNAs obtained from the suppressive subtractive hybridization. Alternatively, the samples could be applied to currently available commercial arrays from Incyte, Affymetrix, Research Genetics, and other commercial vendors. Alternatively, samples could be applied to proteomic/protein microarrays.

After a pattern of expression has been captured and defined, an injury database can be established for the injury state. Once an injury database is established for the injury state, only one fluorescent dye is necessary to capture the pattern of expression for subsequent samples as the pattern will be compared to the established injury database.

An example of a commercially available microarray is an Affymetrix chip. These arrays are fabricated using spatially patterned, light-directed combinatorial chemical synthesis, and contain hundreds of thousands of oligonucleotides immobilized on the glass surface of the arrays (Affymetrix, Santa Clara, Calif.). For most sequences or EST there are 16 probe 20 mer oligonucleotide pairs, of which 8 a perfect match and 8 are a mismatch where one nucleotide is changed in the middle of the sequence. Each array also contains a number of reference sequences, which after standards are added allows normalization and quantification of the data. The human U95A array is used, having 13000 sequences and EST's.

In an embodiment of the invention, the expression levels of the molecules, captured on the microarray, are ranked from the lowest expressed molecule being assigned a rank of 1 to the most highly expressed molecule. For example, if 100,000 molecules were assessed from a single blood sample, the lowest expressed molecule would be assigned a value of 1 and the most highly expressed molecule a value of 100,000 with every other molecule having a value in between. The ranks of the molecules of individuals with a specific injury or on a specific medication are compared to other individuals with other conditions or to normal healthy controls.

In a further embodiment of the invention, the determination of a pattern of expression further comprises ranking the genes of the captured pattern of expression. The expression levels of the genes, captured on the microarray, are ranked from the lowest expressed gene being assigned a rank of 1 to the most highly expressed gene. For example, if 100,000 genes were assessed from a single blood sample, the lowest expressed gene would be assigned a value of 1 and the most highly expressed gene a value of 100,000 with every other gene having a value in between. The ranks of the genes of individuals with a specific injury or on a specific medication are compared to other individuals with other conditions or to normal healthy controls.

In one embodiment of the invention, microarrays are used to capture the pattern of protein expression. The protein is isolated from either whole blood and/or from white blood cells isolated from whole blood. The protein is then applied to a protein microarray. A protein microarray may be composed of antibodies to all known proteins, antibodies to selected protein subsets, or proteins themselves.

In yet another embodiment of the invention, protein detection is used. Protein detection may include multiple mass spectrophotometric analyses performed in parallel or any other method of detecting hundreds to thousands of proteins at one time from a single blood sample from a single patient. The proteins and antibodies are detected using mass spectrophotometric, fluorescent, radioactive or other techniques and the expression levels of each protein assessed in a manner analogous to detection of multiple RNA species on current oligonucleotide and cDNA microarrays.

In yet another embodiment of the invention, the determination of a pattern of expression further comprises ranking the proteins of the captured pattern of expression. The expression levels of the proteins, captured on the microarray, are ranked from the lowest expressed protein being assigned a rank of 1 to the most highly expressed protein. For example, if 100,000 proteins were assessed from a single blood sample, the lowest expressed protein would be assigned a value of 1 and the most highly expressed protein a value of 100,000 with every other protein having a value in between. The ranks of the proteins with individuals with a specific injury or on a specific medication are compared to other individuals with other conditions or to normal healthy controls.

Any expression method known in the art may be used to define the pattern of expression captured. A preferred method is the Significance Analysis of Microarrays (SAM) and class prediction, as taught by Tusher, Proceedings National Academy of Sciences, 98: 5116 (2001); Golub et al., Science, 286: 531-537(1999). Other expression methods are available, including neural network modeling, clustering, computer programs, and entropy methods, and could be used as alternatives.

The significance analysis of microarray (SAM) and class prediction may be used to define the pattern of expression captured. The significance analysis of microarrays uses permutations of repeated measurements to estimate the percentage of genes or proteins identified by chance. Once the molecules are identified that are regulated in a specific injury, this set of molecules is said to define the pattern expression for that injury. To determine whether an unknown sample is consistent with the normal pattern of expression or is consistent with the pattern for a specific injury, the following general procedure is followed. The expression value for each molecule in the unknown sample is compared to the expression value in the normal set of molecules and in the injury set of genes or proteins. A class prediction method is then used to determine whether the unknown sample fits the normal or injury pattern. To do this, the expression value for each molecule is determined to be closer to the control or the injury state, and a weighted vote is made for each molecule for the injury pattern. The diagnosis of the injury is made if PS>0.3 when PS is the prediction strength, defined as PS=(Vw−V_{L})/(Vw+V_{L}). If there is no difference between the samples, then PS will equal zero and the sample would fall in the class of the control or healthy blood sample. If PS>0.3, then the sample would be classified as the injury state.

In one embodiment of the invention, the most regulated genes or proteins for a given condition that had the lowest variance may be identified using SAM analysis for various medical, neurological, genetic and other conditions. These regulated genes or proteins may be used to define a pattern for each condition, a class prediction, that would be used to analyze unknown samples to determine whether they would fit the pattern for a specific disease or condition or not with a 90, 95 or 99% confidence level.

Once the pattern of expression is captured and defined, the pattern of expression exhibited by the obtained blood cells is compared to an injury database to assess the injury. This database may comprise a pattern of expression or multiple patterns of expression based on a specific organ, a specific injury cause or disease, or combinations thereof. Further, the database may be a commercially available database or a database created from the pattern of expression captured and defined by the obtained blood cells.

In one embodiment of the invention, injury databases for hypoxia, status epilepticus and hypoglycemia, are prepared using blood cell samples. These databases are used to assess the injury of an individual based on the comparison between the pattern of expression of the individual and pattern of expression of the database.

The embodiments, as set forth above, can be used for any injury as the blood expression will differ with each and every different injury and the database will remain constant.

### EXAMPLES

In the examples and throughout the present specification, parts and percentages are by weight unless otherwise indicated.

### Example 1

This example demonstrates the use of the claimed invention to assess hypoxia, status epilepticus, hypoglycemia, ischemic stroke, and hemorrhagic stroke in individuals. One day after hypoxia, status epilepticus, hypoglycemia, ischemic stroke, and hemorrhagic stroke are produced in adult rats, RNA or protein is isolated from the blood cells and from the brains of these animals. Suppressive-subtractive hybridization is performed on the isolated RNA or protein. The clones, obtained from the suppressive-subtractive hybridization, or the isolated RNA or protein are sequenced. The pattern of genes or proteins expressed in the blood cells following each of these types of injury—hypoxia, status epilepticus, hypoglycemia, ischemic stroke, and hemorrhagic stroke is captured. The pattern of gene or protein expression is defined using an expression method, which then forms a genomic or proteomic organ injury database, which is used in assessing injury in the individuals.

More specifically, adult Sprague Dawley rats (300-350 gm males) are housed in a fully AAALAC accredited Animal Research Facility. All animals are examined upon receipt and any animals with symptoms of disease or other problems are sacrificed. Animals are fed ad libitum, with fresh food and water provided several times weekly. Cages are cleaned on a regular schedule.

A custom hypoxia chamber is constructed comprising four identical chambers wherein inlet and outlet air is controlled and monitored. Any oxygen concentration (0-100%, by volume) can be achieved using computer controlled valves and pumps. The inlet and outlet oxygen concentration in each chamber is measured continuously, as is carbon dioxide, temperature and humidity. The oxygen concentrations can be ramped up or down over any period of time (seconds to hours). Generally, the 8%, by volume, oxygen concentration is ramped down over 30 minutes, and the animals remain at 8% oxygen for 6 hours, after which the oxygen is ramped back up to 21%.

Status epilepticus is produced by intraperitoneally injecting a glutamate analogue/excitotoxin, kainic acid (10 mg/kg i.p.). Animals with kainate-induced seizures are observed following drug administration to ensure that they continue to have complex seizures over a 30 minute period. Animals with seizures longer than 30 minutes and that have neuronal injury demonstrated histologically are included in the study. Animals injected with kainic acid have diffuse neuronal injury 24 hours later.

Regular insulin (20U sq) is used to induce systemic hypoglycemia. The animals are injected subcutaneously with 10U regular insulin and go into a coma for several hours. The severe hypoglycemia causes severe diffuse neuronal injury. Animals remain hypoglycemic for a period of 4 hours. The hypoglycemia is then reversed with repeated injections of 25% dextrose (25 cc) given every half hour for two hours as needed. Prolonged hypoglycemia is required to produce neuronal injury in the brain and other organs. These periods of hypoglycemia induce glucose-regulated protein 75 (GRP75) and other glucose regulated proteins in brain and other organs such as the liver and other tissues.

Ischemic stroke is produced by anesthetizing adult rats with isoflurane. A ventral neck incision is made, and the common carotid artery is isolated. The external carotid artery is ligated, and a 4-0 nylon suture advanced into the external carotid artery and then up the internal carotid artery to the bifurcation of the middle and anterior cerebral arteries. The suture is left in place for two hours to produce an infarction (stroke) in the distribution of the middle cerebral artery. Control animals for the stroke are called “sham” animals. These animals are anesthetized, have the neck incision performed, and arteries isolated, but do not have the suture inserted into the artery and do not have an ischemic stroke.

Hemorrhagic stroke is produced by anesthetizing adult rats with isoflurane. The scalp is incised and a burr hole drilled 0.5 mm anterior and 4 mm lateral to bregma. A 25 gauge needle was used to deliver 50 μl of lysed arterial blood 4 mm into the right striatum. The hemorrhage results in cell death around the margins of the hemorrhage.

Untouched, control animals are not injected or touched prior to the experiment. These animals remain awake, do not undergo surgery, but are housed and treated like the other animals described above.

All animals are allowed to survive for 24 hours following each treatment. At that time they are deeply anesthetized with ketamine (100 mg/kg) and xylazine (20 mg/kg) given intraperitoneally. Once anesthetized, the chest is opened and a direct cardiac puncture performed with a syringe and 10 cc of blood is aspirated. Immediately following removal of the blood, the animal is decapitated while deeply anesthetized and the brain removed.

The blood from the animals from the hypoxia group is pooled, as is blood from the animals from the status epilepticus group, the animals from the hemorrhagic stroke group, the animal from the ischemic stroke group, and the animals from the hypoglycemia group. The blood from the untouched control and the sham-operated control animals is pooled as well. White blood cells are separated on a FICOLL® gradient, and the RNA from each pooled group is extracted with Trizol reagent. Subtractive hybridizations are then performed using commercially available kits (ClonTech) to obtain several separate subtraction libraries: control versus hypoxia blood; control versus status epilepticus blood; control versus hypoglycemic blood; control versus ischemic stroke blood; and control versus hemorrhagic stroke blood. Generally there are about 500 to about 1000 clones for each subtraction.

Suppressive subtractive hybridization (SSH) is based on a form of PCR that permits exponential amplification of cDNAs that differ in abundance, whereas amplification of RNAs of similar abundance in the control and experimental populations is suppressed. Alternatively, Representational Difference Analysis (RDA) may be used for performing library subtractions.

Poly A+RNA from the control bloods (“driver” or “control”) and the hypoxic, hypoglycemic, ischemic stroke, hemorrhagic stroke, or status epilepticus bloods (“tester” or “experimental”) is made, and then quantified on a formaldehyde gel. Each sample is concentrated to a range of from about 1 to about 2 μg/ml. Double stranded (ds) cDNAs are prepared from the two poly A+RNA samples by reverse transcription. Second strand cDNA synthesis is then performed and the ds cDNAs are digested with a four-base cutting enzyme (Rsa I) that yields blunt ends. The cut ds cDNAs are digested with a four-base cutting enzyme (Rsa I) that yields blunt ends. The cut ds cDNAs are analyzed on a 1%, by weight, agarose gel.

Following this, the tester ds cDNA pool is divided into two equal portions, and the ds cDNA in one portion is ligated with adaptor 1 and the cDNA in the other portion is ligated with adaptor 2 using T4 DNA ligase. Since the ends of the adaptors do not have a phosphate group, only one strand of each adaptor attaches to the 5′ ends of the cDNA. Importantly, the two adaptors (1 and 2R) share a stretch of common sequences that allows them to anneal with each other during PCR. Following successful ligation of the adaptors, hybridization is performed with excess “driver” added to each “tester” sample. The samples are heat denatured and allowed to anneal. The concentration of high and low abundance cDNAs are equalized in the adaptor-ligated population of cDNAs. The cDNAs are equalized due to second-order hybridization kinetics for these differently expressed cDNAs (ClonTech). There is exponential amplification of rare cDNAs in the “tester” samples. During the second hybridization, the two “tester” samples ligated with adaptor 1 and 2R, and the freshly denatured “driver” sample are mixed without denaturing. Only the equalized and subtracted single stranded (ss) tester molecules can re-associate and form double stranded hybrids. The ends (site of different adaptors) are then filled in and these new hybrids are amplified by PCR. Molecules missing the primer annealing sites (adaptor 1 and 2R) cannot be amplified due to suppression of PCR.

The subtracted library is ligated into the T/A cloning vector (Invitrogen, Inc.) and electroporated into phage-resistant bacterial cells (DH10B), which are then stored in glycerol at −80° C. An aliquot (100 μl) of the library is plated on a LB agar plate with the appropriate antibody for the purpose of determining the titer of the library. The T/A cloning vector has a B-galactosidase site that provides the mechanism for color (blue vs white) selection of bacterial colonies that contain a subtracted clone. Positive colonies are inoculated in 96-well plates with antibiotic and 10% glycerol and stored at −80° C. This becomes the original copy of the library. Several controls are performed to help ensure that the procedure worked properly. First, from about 60 to about 80 randomly selected clones are examined on 2% agarose gels to show that the inserts are of the appropriate sizes ranging from about 0.3 to about 1 kb, and that they are of differing sizes and therefore unique. PCR for G3PDH (gyceraldhyde-3-phosphate dehydrogenase) is performed on the subtracted and unsubtracted libraries to ensure that the ubiquitously expressed and unregulated G3PDH is not expressed in the subtracted library.

Clones that show a two fold or greater induction by hypoxia, hypoglycemia ischemic stroke, hemorrhagic stroke, or status epilepticus in the five subtracted libraries are sequenced and compared to currently available rat sequences (GeneBank). The cloned sequences are also subjected to BLAST (basic local alignment search tool, GenBank database) to determine if they match the sequences of known genes. BLAST is a computer program used to search databases to determine if a sequence is similar to that of known or previously cloned genes.

Once a sufficient number of clones are sequenced and their identity determined, genes are selected for further study based upon their expression with each type of injury. For example, glucose regulated genes are induced with hypoglycemia and not with hypoxia and status epilepticus. Hypoxia-inducible factor and its hypoxia-inducible target genes are induced with hypoxia and not with hypoglycemia or status epilepticus. Catecholamine-related genes, like alpha-adrenergic and beta adrenergic-receptors, are induced to a greater extent following status epilepticus as compared to hypoxia or hypoglycemia. Once candidate clones are identified, then the clones are used to perform Northern blots on RNA from bloods of the hypoxic, hypoglycemic, status epilepticus, ischemic stroke, hemorrhagic stroke and control groups. Alternatively, PCR is performed on each sample and the PCR products sequenced to confirm gene induction for each group. Each clone is then used to produce a spot on a microarray.

Northern blots are performed to confirm the specificity of the clones for each gene and to quantify RNA induction. After isolation of RNA, it is incubated with DNase (5 U/ml; Promega) and RNAsin (200 U/ml; Promega) at 37° C. for 30 min. The RNA is ethanol precipitated, dissolved in water and the OD260/280 determined. Four micrograms of RNA are electrophoresed in a 1.5% agarose gel containing 1×MOPS and 7% paraformaldehyde and transferred to a nylon membrane (Nytran, Sleicher and Schuell, Keene, N.H.) for a period of from about 12 to about 18 hours. The RNA is cross-linked to the membrane with UV light at 254 nm (Stratalinker, Stratagene, Calif.). The membrane is stained with 0.02% methylene blue and the position of the 18S and 28S bands marked on the membrane. It is then pre-hybridized at 42° C. for about 1 hour with a mixture of 6×SSC, 0.1% SDS, 10×Denhardt's reagent and 50 μg/ml heat denatured salmon sperm DNA. Clones are labeled using TdT (Gibco BRL) with ³²P-dATP (DuPont-NEN Research Products) and membranes are hybridized at 37° C. overnight in 6×SSC, 1% SDS and 1-4×10⁶ cpm/ml of the labeled probe. After hybridization, the membranes are washed to a maximum stringency of 6×SSC and 0.1% SDS (sodium dodecyl sulfate) at 55° C. The membranes are then covered with Kodak SB5 autoradiographic film for a period of from about 4 to about 12 hours and developed in Kodak GBX developer. Blots are quantified using an MCID (St. Catherine's, Ontario, Canada) image analysis system.

The fabricated microarray is used to capture the pattern of expression in the injury states of hypoxia, status epilepticus, hypoglycemia, ischemic stroke, and hemorrhagic stroke. An expression method defines the pattern of expression and the pattern of expression is compared to an injury database to assess the injury.

### Example 2

This example demonstrates the use of the claimed invention to assess hypoxia, status epilepticus, hypoglycemia, ischemic stroke, and hemorrhagic stroke. One day after hypoxia, status epilepticus, hypoglycemia, ischemic stroke, and hemorrhagic stroke are produced in adult rats, RNA or protein is isolated from the blood cells and from the brains of the animals described in Example 1. The pattern of genes or proteins expressed in the blood cells following each of these types of injury—hypoxia, status epilepticus, hypoglycemia, ischemic stroke, and hemorrhagic stroke is captured on a commercially available microarray (Affymetrix chip). The pattern of gene or protein expression is defined using an expression method, which then forms a genomic or proteomic organ injury database, which is used in assessing injury.

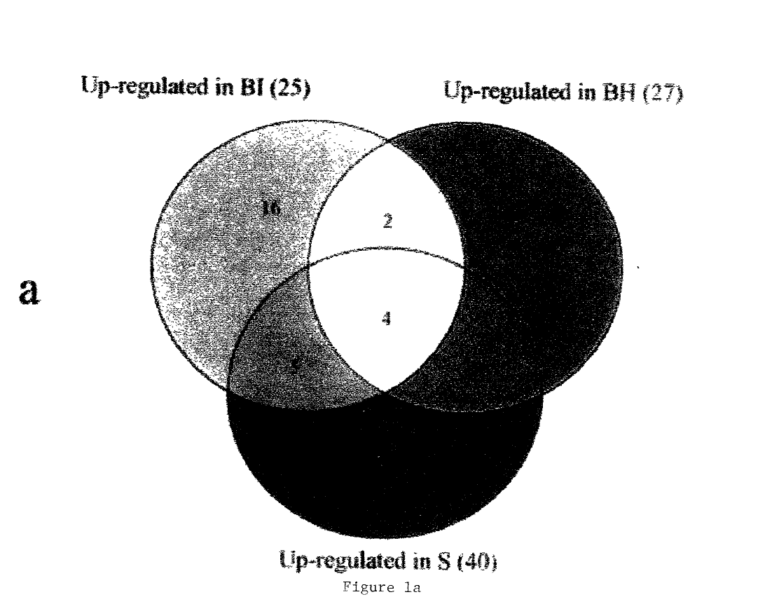

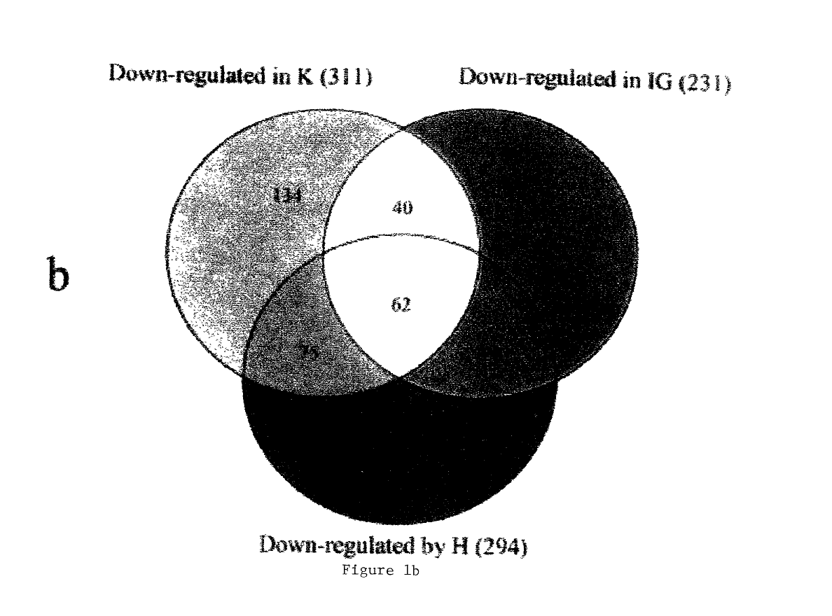

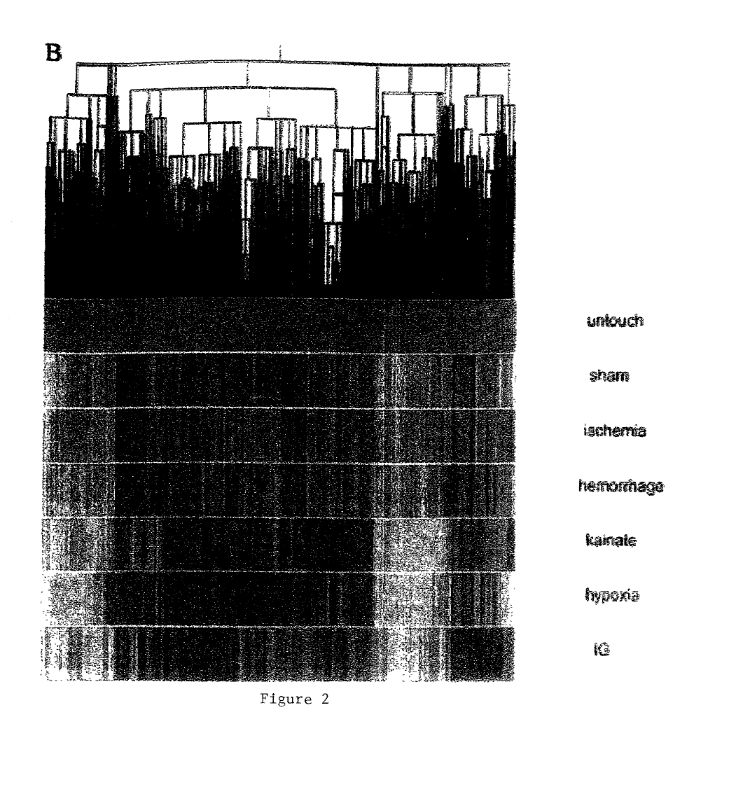

The data below demonstrates the pattern of gene expression in the blood cells and in the brain following specific pathological insults using genomic profiles based on commercially available microarrays. The data demonstrate how a pattern of gene expression is derived, and that the patterns of gene expression for the different pathological states are different from each other. The tables give lists of genes induced in blood and in the brain of animals exposed to hypoxia, stroke, and status epilepticus as compared with untouched control or sham operated control animals. As shown in FIGS. 1a and 1b, many genes upregulated or downregulated by each experimental condition were modulated in two or more groups. FIG. 2 presents a cluster analysis of the pattern of expression obtained from individuals with kainate, insulin-glucose, hypoxia, brain ischemia, brain hemorrhage, as compared to sham surgery and untouched control individuals.

For the tables of genes induced in the blood, the genome expression of blood in the hypoxic animals (3 animals) was compared to the genome expression of blood in untouched control animals (3 animals). The genome expression of blood in the animals with status epilepticus (3 animals) was compared to the genome expression of blood in the untouched control animals (3 animals). The genome expression of blood in the animals with stroke (3 animals) was compared to the genome expression of blood in the sham operated control animals (3 animals). In each case the accession number of the gene and the fold change in gene expression is given—with a maximum estimate and a minimum estimate.

Tables 1 to 4 set forth lists of genes induced in the blood in the different conditions. Tables 5 and 6 set forth lists of genes induced in the brain in the different conditions. Note that the genes induced in the blood are different from the genes induced in the brain. Therefore, different organs express different genes. In addition, the genes induced by hypoxia in the blood are different from the genes induced by hypoxia in the brain. That is, the same stimulus induces different genes in different organs. Lastly, even though similar genes are induced in the brain by ischemia (stroke) and kainic acid-induced seizures, there are many differences in the gene expression between the two. Therefore, the pattern of gene expression in the brains of ischemic animals is distinctive from the pattern of expression of the kainate animals, and this pattern can be used to diagnose the different conditions of stroke and status epilepticus, even though many of the same genes are induced in the two conditions.

Table 1 sets forth genes induced in the blood of rats 24 hours following 6 hours of 8% hypoxia (n=3 rats) as compared with genes expressed in the blood of untouched control rats (n=3 rats). The accession number of the gene is given, the name of the gene is given where known, the average fold induction is given, as well as the minimum fold induction is given for each gene. A number of the genes are ESTs that have not yet been subjected to a BLAST search. This list represents the number of genes induced on arrays that contained 8000 genes.

TABLE 1

| Accession No. | Name | Average | Minimum |
|---|---|---|---|
| X62950mRNA_f_at | pBUS30 with repetitive | 10 | 4.8 |
| | elements | | |
| rc_AA891933_at | | 9 | 1.9 |
| X06827_at | porphobilinogen deaminase | 7.1 | 4.1 |
| rc_AA894273_at | | 6.1 | 2.7 |
| X63675_at | Pim-1 | 6 | 1.8 |
| D13978_s_at | argininosuccinate lyase | 5.1 | 1.9 |
| X62325cds_r_at | T cell receptor V-alpha | 5 | 1.8 |
| | J-alpha | | |
| rc_AA891737_at | | 5 | 1.6 |
| rc_AA891920_at | | 4.9 | 2.7 |
| S65555_g_at | gamma-glutamylcysteine | 4.5 | 2.1 |
| | synthetase light chain | | |
| rc_AI233261_i_at | | 4.4 | 1.5 |
| X06827_g_at | porphobilinogen deaminase | 4.3 | 2’ |
| rc_AA800745_at | | 4.3 | 1.5 |
| X17053mRNA_s_at | Rat immediate-early serum- | 4.2 | 3.9 |
| | responsive JE gene | | |
| rc_H33723_at | | 4.1 | 2.6 |
| S65555_at | gamma-glutamylcysteine | 4.1 | 1.9 |
| | synthetase light chain | | |
| U39875_at | EF-hand Ca2+-binding | 4 | 1.8 |
| | protein p22 | | |
| rc_AI059042_at | | 4 | 1.7 |
| M91234_f_at | VL30 element | 3.9 | 2.4 |
| U73030_at | pituitary tumor | 3.9 | 1.6 |
| | transforming gene (PTTG) | | |
| Y13275_at | D6.1A protein | 3.9 | 1.5 |
| M59936cds_at | connexin-31 | 3.8 | 2.1 |
| rc_AA852046_s_at | | 3.8 | 2.1 |
| rc_AA852046_s_at | | 3.8 | 2.1 |
| rc_AI145680_s_at | | 3.8 | 1.6 |
| rc_AI045315_f_at | | 3.8 | 1.4 |
| M15474cds_s_at | alpha-tropomyosin gene | 3.7 | 2.4 |
| AF102552_s_at | 270 kDa ankyrin G isoform | 3.7 | 1.7 |
| M91235_f_at | VL30 element | 3.6 | 2.4 |
| U07201_at | asparagine synthetase | 3.5 | 2 |
| AB015194_at | 50 kD glycoprotein (Rh50) | 3.5 | 1.8 |
| U25650_f_at | low affinity nerve growth | 3.5 | 1.4 |
| | factor receptor precursor | | |
| | (LNGFR) | | |
| X17053cds_s_at | Rat immediate-early serum- | 3.4 | 2.1 |
| | responsive JE gene | | |
| Y00350_at | uroporphyrinogen | 3.4 | 1.9 |
| | decarboxylase | | |
| rc_AA891880_g_at | | 3.4 | 1.3 |
| rc_AI235890_s_at | | 3.3 | 2.5 |
| rc_AI235890_s_at | | 3.3 | 2.3 |
| AB000199_at | cca2 | 3.3 | 1.3 |
| M62388_at | ubiquitin conjugating- | 3.2 | 1.8 |
| | protein | | |
| X89225cds_s_at | L-like neutral amino acid | 3.2 | 1.5 |
| | transport activity protein | | |
| rc_AA858607_at | | 3.2 | 1.4 |
| X82396_at | cathepsin B | 3.1 | 2.3 |
| X62660mRNA_g_at | glutathione transferase | 3.1 | 1.3 |
| | subunit B | | |
| M60666_s_at | alpha-tropomyosin 2 | 3 | 1.7 |
| rc_AA926149_g_at | | 3 | 1.6 |
| AF076856_s_at | small espin | 2.9 | 1.8 |
| rc_AA892897_at | | 2.8 | 1.7 |
| D90401_g_at | dihydrolipoamide | 2.8 | 1.4 |
| | succinyltransferase | | |
| M34134_s_at | brain alpha-tropomyosin | 2.8 | 1.4 |
| | (TMBr-2) | | |
| rc_AA799680_at | | 2.8 | 1.4 |
| rc_AI029920_s_at | | 2.8 | 1.3 |
| rc_AA891107_at | | 2.7 | 1.6 |
| rc_AI235585_s_at | | 2.7 | 1.6 |
| X67948_at | channel integral membrane | 2.7 | 1.6 |
| | protein 28 | | |
| AF067790_s_at | palmitoyl-protein | 2.7 | 1.4 |
| | thioesterase | | |
| M89945mRNA_g_at | Rat farnesyl diphosphate | 2.7 | 1.4 |
| | synthase gene | | |
| rc_AA819793_at | | 2.6 | 1.8 |
| J02592_s_at | glutathione S-transferase | 2.6 | 1.6 |
| | Y-b subunit | | |
| rc_AA893590_at | | 2.6 | 1.6 |
| AF990113_g_at | AMPA receptor binding | 2.6 | 1.4 |
| | protein | | |
| M89945mRNA_at | Rat farnesyl diphosphate | 2.5 | 1.6 |
| | synthase gene | | |
| rc_AI180442_at | | 2.5 | 1.5 |
| D63774_at | keratin 14 | 2.5 | 1.3 |
| rc_AA818025_at | | 2.4 | 1.3 |
| rc_AI014094_at | | 2.4 | 1.3 |
| D86215_at | brain mRNA for NADH- | 2.3 | 2.1 |
| | ubiquinone oxidoreductase | | |
| rc_AA874827_at | | 2.3 | 1.6 |
| rc_AA946368_at | | 2.3 | 1.6 |
| U82623_g_at | cytocentrin | 2.3 | 1.6 |
| X12554cds_s_at | heart cytochrome c oxidase | 2.3 | 1.4 |
| | subunit VIa | | |
| AJ009698_g_at | embigin protein | 2.3 | 1.3 |
| D10026_s_at | glutathione S-transferase | 2.2 | 1.7 |
| rc_AA851403_g_at | | 2.2 | 1.5 |
| U67138_at | PSD-95/SAP90-associated | 2.2 | 1.4 |
| | protein-2 | | |
| D38036_at | Truncated TSH receptor | 2.2 | 1.3 |
| rc_AA892805_g_at | | 2.2 | 1.3 |
| rc_AI013513_at | | 2.2 | 1.3 |
| rc_AA851887_s_at | | 2.1 | 1.6 |
| D13120_s_at | ATP synthase subunit d | 2.1 | 1.4 |
| rc_AA892888_at | | 2.1 | 1.4 |
| U82623_at | cytocentrin | 2.1 | 1.4 |
| D16478_at | mitochondrial long-chain | 2.1 | 1.3 |
| | enoyl-CoA hydratase | | |
| rc_AA799612_at | | 2.1 | 1.3 |
| AF029240_at | MHC class Ib RT1.S3 | 2 | 1.4 |
| J05022_at | peptidylarginine deiminase | 2 | 1.4 |
| rc_AI231472_s_at | | 2 | 1.4 |
| rc_AA866477_at | | 2 | 1.3 |
| rc_AA875107_at | | 2 | 1.3 |
| rc_AI105050_at | | 2 | 1.3 |
| rc_AA925752_at | | 2 | 1.1 |
| AF050663UTR#1_at | norvegicus activity and | 1.9 | 1.5 |
| | neurotransmitter-induced | | |
| | early gene | | |
| X53363cds_s_at | calreticulin | 1.9 | 1.5 |
| S78154_at | inwardly rectifying ATP- | 1.9 | 1.4 |
| | regulated K+ channel | | |
| U24489_at | tenascin-X | 1.9 | 1.4 |
| X63722cds_s_at | vascular cell adhesion | 1.9 | 1.4 |
| | molecule-1 (VCAM-1) | | |
| D13212_s_at | N-methyl-D-aspartate | 1.9 | 1.3 |
| | receptor subunit | | |
| | (NMDAR2C) | | |
| D78308_g_at | calreticulin | 1.9 | 1.3 |
| AF017437_at | integrin-associated protein | 1.8 | 1.5 |
| | form 4 (IAP) | | |
| X03369_s_at | beta-tubulin T beta15 | 1.8 | 1.5 |
| D45254_g_at | cellular nucleic acid | 1.8 | 1.4 |
| | binding protein (CNBP) | | |
| rc_AI146195_at | | 1.8 | 1.4 |
| AF020618_at | progression elevated gene 3 | 1.8 | 1.3 |
| | protein | | |
| AF060174_at | synaptic vesicle protein 2C | 1.8 | 1.3 |
| | (SV2C) | | |
| D10587_at | 85 kDa sialoglycoprotein | 1.8 | 1.3 |
| | (LGP85) | | |
| rc_AA799887_s_at | | 1.8 | 1.3 |
| rc_AA859957_at | | 1.8 | 1.3 |
| X80395cds_s_at | rVAT gene | 1.8 | 1.3 |
| rc_AA892260_at | | 1.7 | 1.4 |
| AF017437_at | integrin-associated protein | 1.7 | 1.3 |
| | form 4 (IAP) | | |
| AF073839_s_at | bithoraxoid-like protein | 1.7 | 1.3 |
| Rc_AI169631_s_at | | 1.7 | 1.3 |
| U36444cds#1_at | PCTAIRE-1 protein kinase | 1.7 | 1.3 |
| L38437_at | NADH ubiquinone | 1.6 | 1.3 |
| | oxidoreductase subunit | | |
| | (IP13) gene | | |
| rc_AI112237_at | | 1.6 | 1.3 |
| rc_AA893690_g_at | | 1.5 | 1.3 |

Table 2 sets forth genes induced in the blood of rats 24 hours following kainate induced seizures (n=3 rats) as compared with genes expressed in the blood of untouched control rats (n=3 rats). The accession number of the gene is given, the name of the gene is given where known, the average fold induction is given, as well as the minimum fold induction is given for each gene. A number of the genes are ESTs that have not yet been subjected to a BLAST search. This list was shortened to show only those genes induced at least 2.8 fold. Over 100 genes were induced following kainate on arrays that contained over 8000 genes.

TABLE 2

| Accession No. | Name | Average | Minimum |
|---|---|---|---|
| D84485_at | PMSG-induced ovarian | 11.4 | 3.1 |
| | mRNA | | |
| M96159_at | adenylyl cyclase type V | 10 | 2.9 |
| Rc_AA955182_g_at | | 9 | 2.3 |
| AF045464_s_at | | 6.5 | 2.5 |
| X76697_at | B7 antigen | 5.7 | 2.5 |
| D89863_g_at (M-ras) | M-Ras | 5.6 | 2.3 |
| U66566_at | receptor type protein | 5.5 | 4.3 |
| | tyrosine phophatase psi | | |
| L81138exon | Rps2r gene | 5.5 | 2.3 |
| AF079162_at | patched (ptc) | 5.4 | 3.2 |
| Rc_AA894273_at | | 5.2 | 2.8 |
| Rc_AA799614_at | | 4.7 | 2.5 |
| AF102552_s_at | ankyrin G isoform | 4.6 | 2.4 |
| M91234_f_at | VL30 element | 4.4 | 2.5 |
| L42855_at | RNA polymerase II | 4.32 | 3.4 |
| | transcription factor SIII | | |
| | p18 subunit | | |
| Rc_AA852046_s_at | | 4.3 | 2.5 |
| AF027571_s_at | phospholipase C-beta 4 | 4.15 | 2.5 |
| | isoform (PLC-b4) | | |
| Rc_AI104924_f_at | | 4.1 | 3.3 |
| U73030_at | | 4.1 | 2.4 |
| Rc_AA925529_at | | 4 | 3 |
| Rc_AA891828_at | | 4 | 2.6 |
| M91235_f_at | VL30 element | 3.9 | 3 |
| L81136cds_f_at | Rps2r1 preliminary DNA | 3.9 | 2.7 |
| X06827_at | porphobilinogen deaminase | 3.6 | 3 |
| X60675_at | interleukin 10 | 3.6 | 2.3 |
| Z28351exon_s_at | 25-hydroxyvitamin D3 | 3.5 | 2.3 |
| | 24-hydroxylase | | |
| AF091563_i_at | isolate QIL-LD1 olfactory | 3.4 | 2.4 |
| | receptor | | |
| rc_AI102562_at | | 3 | 2.4 |
| S54212_at | ciliary neurotrophic factor | 2.8 | 2.6 |
| | receptor alpha | | |

Table 3 sets forth genes induced in the blood of rats 24 hours following a stroke produced by filament occlusion of the middle cerebral artery (n=3 rats) as compared with genes expressed in the blood of sham operated control rats (n=3 rats). The accession number of the gene is given, the name of the gene is given where known, the average fold induction is given, as well as the minimum fold induction is given for each gene. A number of the genes are ESTs that have not yet been subjected to a BLAST search. This list was produced from arrays that contained over 8000 genes.

TABLE 3

| Accession No. | Name | Average | Minimum |
|---|---|---|---|
| X52196cds_at | five-lipoxygenase activating | 9.5 | 1.7 |
| | protein (FLAP) | | |
| rc_AA866444_s_at | | 8.8 | 2.6 |
| Rc_AA892851_at | | 5.6 | 3.9 |
| rc_H31722 | | 5.4 | 2 |
| L18948_at | intracellular calcium- | 4.1 | 1.7 |
| | binding protein (MRP14) | | |
| rc_AA849036 | | 4 | 2.5 |
| rc_AI043796_s_at | | 3.9 | 2.4 |
| D89093_at | cGMP-bindmg cGMP- | 3.6 | 1.8 |
| | specific phosphodiesterase | | |
| AF023621_at | sortilin | 3.5 | 2 |
| rc_AI639246_at | | 3.2 | 1.7 |
| Rc_AA957003_at | | 3.2 | 1.6 |
| L00603_at | vesicular monoamine | 3 | 2.4 |
| | transporter | | |
| U13396_at | protein-tyrosine kinase | 3 | 2.1 |
| | (JAK2) | | |
| M64986_g_at | amphoterin mRNA | 3 | 1.5 |
| L11319_at | five-lipoxygenase activating | 2.8 | 1.5 |
| | protein (FLAP) | | |
| rc_AA892851_g_at | | 2.7 | 2.3 |
| X78605_at | rab4b mRNA for ras- | 2.7 | 2.3 |
| | homologous GTPase | | |
| U49930_g_at | ICE-like cysteine protease | 2.7 | 1.6 |
| | (Lice) | | |
| rc_AA893534_at | | 2.6 | 1.8 |
| D17521_at | protein kinase C-regulated | 2.6 | 1.7 |
| | chloride channel | | |
| U27201_at | tissue inhibitor of | 2.6 | 1.6 |
| | metalloproteinase 3 | | |
| | (TIMP-3) | | |
| M55532_at | carbohydrate binding | 2.5 | 1.8 |
| | receptor | | |
| D13962_g_at | neuron glucose transporter | 2.5 | 1.4 |
| | (GLUT3) | | |
| rc_AA893664 | | 2.3 | 1.8 |
| AJ000557cds_s_at | Janus protein tyrosine | 2.2 | 1.6 |
| | kinase 2, JAK2 | | |
| rc_AA875206_at | | 2.2 | 1.5 |
| D84346_s_at | Nap1 protein | 2.2 | 1.4 |
| rc_AA800275_at | | 2.2 | 1.4 |
| rc_AI171962_s_at | | 2.2 | 1.4 |
| S70011_g_at | tricarboxylate carrier | 2.1 | 1.8 |
| AF084186_s_at | alpha-fodrin (A2A) | 2.1 | 1.7 |
| L25387_g_at | phosphofructokinase C | 2.1 | 1.6 |
| | (PFK-C) | | |
| rc_AA892049_at | | 2.1 | 1.4 |
| rc_AI638939_at | | 2.1 | 1.4 |
| U09631_at | VIP2 vasoactive intestinal | 2.1 | 1.4 |
| | peptide receptor | | |
| M93017_at | Rat alternatively spliced | 2.1 | 1.3 |
| | mRNA | | |
| rc_AA799402_at | | 2 | 1.8 |
| X78949_at | prolyl 4-hydroxylase alpha | 2 | 1.7 |
| | subunit | | |
| rc_AA799650_at | | 2 | 1.6 |
| rc_AA859520_at | | 2 | 1.6 |
| U41164_at | Cys2/His2 zinc finger | 2 | 1.6 |
| | protein (rKr1) | | |
| X63995_at | NTT | 2 | 1.6 |
| L01793_at | glycogenin | 2 | 1.3 |
| rc_AA891732_at | | 1.9 | 1.5 |
| rc_AA892511_at | | 1.9 | 1.5 |
| rc_AI230778_at | | 1.9 | 1.5 |
| AF099093_g_at | ubiquitin-conjugating | 1.9 | 1.4 |
| | enzyme UBC7 | | |
| rc_AA893217_at | | 1.9 | 1.4 |
| rc_AA956958_at | | 1.9 | 1.4 |
| rc_AI045794_at | | 1.9 | 1.3 |
| rc_AA799637_at | | 1.8 | 1.6 |
| rc_H31610_at | | 1.8 | 1.5 |
| X78606_at | rab28 mRNA for ras- | 1.8 | 1.5 |
| | homologous GTPase | | |
| rc_AA875594_s_at | | 1.8 | 1.4 |
| rc_AI171506_g_at | | 1.8 | 1.4 |
| S70011_at | tricarboxylate carrier | 1.8 | 1.4 |
| rc_AA893002_at | | 1.8 | 1.3 |
| X61295cds_s_at | L1 retroposon, ORF2 | 1.8 | 1.3 |
| | mRNA | | |
| rc_AA799570_at | | 1.7 | 1.5 |
| rc_AA874934_at | | 1.7 | 1.5 |
| rc_AA892642_at | | 1.7 | 1.4 |
| X63253cds_s_at | serotonin transporter | 1.7 | 1.4 |
| rc_AA800787_at | | 1.7 | 1.3 |
| rc_AA891068_f_at | | 1.7 | 1.3 |
| rc_AA892014_r_at | | 1.7 | 1.3 |
| rc_AA892496_at | | 1.7 | 1.3 |
| rc_AA893237_at | | 1.7 | 1.3 |
| rc_AI228247_at | | 1.7 | 1.3 |
| rc_AI639162_at | | 1.6 | 1.5 |
| X73371_at | Fc gamma receptor | 1.6 | 1.4 |
| rc_AA801286_at | | 1.4 | 1.3 |
| U57050_g_at | hypertension-related mRNA | 1.3 | 1.3 |

Table 4 sets forth genes induced in the blood of rats 24 hours following the sham control operation (n=3 rats) as compared with genes expressed in the blood of untouched control rats (n=3 rats). The accession number of the gene is given, the name of the gene is given where known, the average fold induction is given, as well as the minimum fold induction is given for each gene. A number of the genes are ESTs that have not yet been subjected to a BLAST search. This list was produced from arrays that contained over 8000 genes.

TABLE 4

| Accession No. | Name | Average | Minimum |
|---|---|---|---|
| M58040_at | transferrin receptor | 5.8 | 3 |
| D50564_at | mercaptopyruvate | 5 | 1.55 |
| | sulfurtransferase | | |
| U07201_at | asparagine synthetase | 4 | 3 |
| rc_AA894273_at | | 3 | 1.7 |
| AF087674_at | insulin receptor substrate 2 | 2.9 | 1.9 |
| | (IRS-2) | | |
| rc_AA858607_at | | 2.7 | 1.3 |
| X06827_at | porphobilinogen deaminase | 2.6 | 1.6 |
| D28966_at | prostacyclin receptor | 2.6 | 1.5 |
| rc_AA852046_s_at | | 2.6 | 1.3 |
| E00594cds_at | immunoglobulin E binding | 2.5 | 1.4 |
| | factor activity peptide | | |
| M91235_f_at | VL30 element | 2.4 | 1.8 |
| rc_AA892897_at | | 2.3 | 1.5 |
| M91234_f_at | VL30 element | 2.2 | 1.5 |
| rc_AA819793 at | | 2.1 | 1.7 |
| U12514_at | transcriptional regulator | 2.1 | 1.4 |
| | MSX-2 (MSX-2) | | |
| AF079162_at | patched (ptc) | 2.1 | 1.3 |
| X67948_at | channel integral membrane | 2.1 | 1.3 |
| | protein 28 | | |
| X82396_at | cathepsin B | 2 | 1.6 |
| AB015645_at | G protein-coupled receptor | 1.9 | 1.5 |
| L12384_at | ADP-ribosylation factor 5 | 1.9 | 1.3 |
| AF087696_at | dlg 2 | 1.8 | 1.4 |
| U53486mRNA_s_at | corticotropin releasing | 1.8 | 1.4 |
| | factor receptor | | |
| rc_AA800566_g_at | | 1.8 | 1.3 |
| X12554cds_s_at | heart cytochrome c oxidase | 1.8 | 1.3 |
| | subunit VIa | | |
| X63722cds_s_at | vascular cell adhesion | 1.4 | 1.2 |
| | molecule-1 | | |

The above blood data only catalogues the genes that show an increase of expression in one condition versus the other. Not listed above are an equal number of genes that show down-regulation or decreases following stroke, seizures and hypoxia when compared to controls. The genes that show down regulation are just as important for describing the pattern of gene regulation in blood but are not included the downregulated genes in the above lists for the sake of simplicity. The downregulated genes in the list of hypoxia-regulated genes in brain are set forth below as an example.

The above data show that different genes, for the most part, are induced in the blood cells of rats following stroke, hypoxia and status epilepticus as compared with the controls. In addition, the genes induced in the blood cells of rats following sham control operations differed from the genes expressed in the blood cells of untouched rats. This data suggests that different patterns of expression will occur in the blood depending on the injury or the cause of the injury. The pattern of expression for each injury is distinct and therefore can be used to assess the injury.

In further support, the following Tables 5 and 6 list those genes induced in the brain following stroke, kainic induced seizures, and hypoxia as compared with untouched controls and sham-operated controls. This data supports the concept that gene expression in the brain differs following different types of injury, just as gene expression in the blood differs following different types of injury.

TABLE 5

| | | | Kainic | |
|---|---|---|---|---|
| | | Stroke | Acid | |
| | | Ischemia | Seizure | Hypoxia |
| | | (fold | (fold | (fold |
| Probe Set | Name | change) | change) | change) |
| M86389cds_s | Rat hsp 27 | 361.9 | 309.2 | NC |
| S82649-r- at | Narp + neuronal | 251.8 | 72.5 | NC |
| | activity-regulated | | | |
| | pentraxin | | | |
| rc_AI169327_g_{—} | Tissue Inhibitor | 239 | 186.7 | NC |
| | of | | | |
| | Metalloproteinase | | | |
| z27118cds_s_{—} | Rat hsp 70 | 183.4 | 37.3 | NC |
| aa848563_s_a | heat shock protein | 145 | 27.1 | NC |
| | 70 | | | |
| d00753_at | Rat RNA for | 134.4 | 55.4 | NC |
| | contrapsin-like | | | |
| | protoease | | | |
| | inhibitor | | | |
| | related protein | | | |
| | (CPi-26) | | | |
| m14656_at | osteopontin m | 79.3 | 39 | NC |
| | RNA | | | |
| x17053RNA_s | rat immediate- | 67.2 | 51.3 | NC |
| | early serum | | | |
| | response gene | | | |
| jo2722cds_at | Rat heme | 68.5 | 20.2 | NC |
| | oxygenase gene | | | |
| z75029_s_at | R. norvegicus hsp | 64.6 | 12.3 | NC |
| | 70.2 RNA for | | | |
| | heat shock protein | | | |
| | 70 | | | |
| m36317_s_at | Rat thyrotropin- | 63.5 | 30.4 | NC |
| | releasing | | | |
| | hormone (TRH) | | | |
| | precursor | | | |
| rc_aa998683 | heat shock protein | 60.6 | 50.5 | NC |
| | 27 | | | |
| ab002588_at | glycerol 3- | 53.3 | 52.4 | |
| | phosphate | | | |
| | deyydrogenase | | | |
| m23566exon_s_{—} | alpha-2- | 53.2 | NC | NC |
| | macroglobulin | | | |
| | gene | | | |
| rc_ai045030 | C/EBP | 52 | 21 | NC |
| x07266_cds_s_{—} | Rat RNA for gene | 51.7 | 21.7 | NC |
| | 33 polypeptide | | | |
| af028784RNA | GFAP | 49.7 | 52.2 | NC |
| af025308_f_a | Rattus norvegicus | 44.4 | no | NC |
| | MHC class 1b | | | |
| | antigen (RT1.C1) | | | |
| | gene | | | |
| m61875_s_at | CD44 | 41.8 | 69.4 | NC |
| x76454_at | ri1 RNA | 39.8 | 50.3 | NC |
| rc_aa818604 | | 37.4 | 7.2 | NC |
| s71196RNA_s_{—} | BDNF | 35.8 | NC | NC |
| M23643cds_s_{—} | TRH | 35.1 | 12 | NC |
| x59864RNA_a | Rat ASM15 gene | 34 | 52.2 | NC |
| m26744_at | interleukin 6 | 32.2 | NC | NC |
| | (IL6) RNA | | | |
| L16764_s_at | heat shock rotein | 32.2 | 10.5 | NC |
| | 70 (HSP70) RNA | | | |
| L18948_at_ | intracellular | 30 | NC | NC |
| | calcium-binding | | | |
| | protein (MRP14) | | | |
| | RNA | | | |
| rc_h33003_at | EST | 28.5 | 36.7 | NC |
| s66024_g_at | transcriptional | 28.3 | 2.8 | NC |
| | repressor CREM | | | |
| s66184_s_at | lysyl oxidase | 27.4 | 5.7 | NC |
| m19651_at_ | Fra-1 | 26 | 11.6 | NC |
| u18982_s_at | Fra-2 | 25.9 | NC | NC |
| af039583 | decay- | 24.7 | NC | NC |
| | accelerating | | | |
| | factor | | | |
| x52498cds_at | TGFB-1 | 24.4 | 12.3 | NC |
| J02962_at_ | Rat IgE binding | 24.1 | 27.7 | NC |
| | protein RNA | | | |
| rc_aa893770 | EST | 24.1 | NC | NC |
| U22414_at | macrophage | 23.8 | NC | NC |
| | inflammatory | | | |
| | protein-1alpha | | | |
| | RNA | | | |
| af075383_at | suppressor of | 22.9 | 17.4 | NC |
| | cytokine | | | |
| | signaling-3 | | | |
| | (SOCS-3) RNA | | | |
| U12187_at | ras-related protein | 22.7 | 7.7 | NC |
| | (rad) RNA | | | |
| rc_aa892333 | EST | 21.9 | 10.3 | NC |
| rc_aa893244 | | 21.9 | 12.5 | NC |
| x17053cds_s | Rat immediate- | | | |
| | early serum- | | | |
| | responsive JE | | | |
| | gene | | | |
| U18729_at | cytochrome b558 | 21.4 | 21.9 | NC |
| | alpha-subunit | | | |
| | RNA | | | |
| rc_aa946503 | EST | 21.3 | 9 | NC |
| x59864RNA_g | Rat ASM15 gene | 21.1 | 23.7 | NC |
| rc_aa799396 | EST | 21 | 2.5 | NC |
| U05014_g_at | PHAS-1 RNA | 20.6 | 17.3 | NC |
| af087943_s_a | CD14 | 19.8 | 8.2 | NC |
| M65149_at | Rat CELF RNA | 19.7 | 7.2 | NC |
| L32132_at | Rat lipopoly- | 19.6 | 7.3 | NC |
| | saccharide | | | |
| | binding protein | | | |
| | RNA | | | |
| U09540_at | cytochrome P450 | 19.2 | 15.3 | NC |
| | (CYP1B1) RNA | | | |
| S76758_i_at | BDNF | 18.5 | NC | NC |
| X17163cds_s_ | c-jun | 17.5 | 10.5 | NC |
| U24441_at | gelatinase B | 17.4 | 22.4 | NC |
| rc_ai639363 | rx03855 EST | 17.1 | NC | NC |
| rc_aa799773_at | EST | 16.9? | NC | NC |
| rc_ai179610 | EST | 15.9 | 3.9 | NC |
| af053312_s_a | CC chemokine | 15.7 | 3.4 | NC NC |
| | ST38 precursor | | | |
| s77528cds_s_ | rNFIL-6 = C/ | 15.7 | NC | NC |
| | EBP-related | | | |
| | transcription | | | |
| | factor | | | |
| d88666 | PS-PLA1 | 15.5 | 9.4 | NC |
| rc_ai169327_at | EST | 15.4 | 8.9 | NC |
| M64795_f_at | Rat MHC class I | 15.2 | no | NC |
| | antigen gene | | | |
| x73371_at | Fc gamma | 14.5 | 10.7 | NC |
| | receptor | | | |
| x71898_at | urinary | 14.5 | 8.8 | NC |
| | plasminogen | | | |
| | activator receptor | | | |
| | 1 | | | |
| U42719_at | C4 complement | 14.1 | 20.7 | NC |
| | protein RNA | | | |
| rc_aa891911 | EST | 14 | 8.8 | NC |
| M11597_at | Rat alpha-type | 13.7 | 9.1 | NC |
| | calcitonin gene- | | | |
| | related peptide | | | |
| | RNA | | | |
| L105489_at | Rat heparin- | 13.1 | 10.5 | NC |
| | binding EGF-like | | | |
| | growth factor | | | |
| | RNA | | | |
| X56306_s_at | Rat RNA of | 12.9 | 10.4 | NC |
| | delta-prepro- | | | |
| | tachykilnin-a | | | |
| | splicing variant | | | |
| | of substance P | | | |
| rc_aa893280 | EST | 12.5 | 9.8 | NC |
| AFO13144_at | MAP-kinase | 12.3 | NC | NC |
| | phosphatase | | | |
| | (cpg21) RNA | | | |
| M24067_at | plasminogen | 12.2 | 6.3 | NC |
| | activator | | | |
| | inhibitor-1 | | | |
| | (PAI-1) RNA | | | |
| z54212_at_ | epithelial | 12.2 | 18.9 | NC |
| | membrane | | | |
| | protein-1 | | | |
| af004811 | moesin RNA | 12 | 23.7 | NC |
| d26393exon_s | Rat HK2 gene for | 12 | 5.8 | NC |
| | type II | | | |
| | hexokinase, exon | | | |
| | 1 and promoter | | | |
| | region | | | |
| rc_ai176658 | EST | 11.9 | 12.9 | NC |
| M26745cds_s | Rat interleukin 6 | 11.7 | NC | NC |
| | (IL6) gene | | | |
| x67948_at | channel integral | 11.5 | 8.9 | NC |
| | membrane protein | | | |
| | 28 | | | |
| x03347cds_g_ | FBR-murine | 11.2 | NC | NC |
| | osteosarcoma | | | |
| | provirus genome | | | |
| x13044_g_at | MHC-associated | 11.1 | 9 | NC |
| | invariant chain | | | |
| | gamma | | | |
| u31599 | MHC class II-like | 11.1 | 11.9 | NC |
| | beta chain | | | |
| | (RT1.Dmb) RNA | | | |
| rc_aa800587 | EST | 11 | NC | NC |
| rc_aa859878 | EST | 10.9 | NC | NC |
| Y00396RNA_a | c-myc | 10.8 | 6.9 | NC |
| D15069_s_at | adrenomedullin | 10.8 | NC | NC |
| | precursor | | | |
| rc_ai230255 | EST | 10.7 | NC | NC |
| M31837_at_ | Rat insulin-like | NC | | |
| | growth factor- | | | |
| | binding protein | | | |
| | (IGF-BP3) | | | |
| m11794cds#2 | Rat | 10.6 | 3.8 | NC |
| | metallothionein-2 | | | |
| | and | | | |
| | metallothionein-1 | | | |
| | genes | | | |
| M64785_g_at | Rat vasopressin | 10.4 | | NC |
| | (VP) RNA | | | |
| rc_ai102562 | EST | 10.3 | 2.9 | NC |
| U06434_at | Rat vasopressin | 10.3 | no | NC |
| | (VP) RNA | | | |
| z12298cds | dermatan sulfate | 10.2 | no | NC |
| | proteoglycan-II | | | |
| | (decorin) | | | |
| u92081RNA_s | epithelial cell | 10.2 | 9.8 | NC |
| | transmembrane | | | |
| | protein antigen | | | |
| | precursor | | | |
| | (RT140) gene | | | |
| re_ai009405 | EST | 10.2 | 7.8 | NC |
| D11445exon#1 | Rattus norvegicus | 10.1 | NC | NC |
| | gene for gro, | | | |
| | complete cds | | | |
| | platelet-activating | | | |
| | factor | | | |
| af016047_at | acetylhydrolse | 9.8 | 5.4 | NC |
| | alpha 1 subunit | | | |
| | (PAF-AH alpha | | | |
| | 1) | | | |
| X74565cds_at | TBFII RNA for | 9.8 | 11.3 | NC |
| | polypyrimidine | | | |
| | tract binding | | | |
| s66024_at | transcriptional | 9.7 | 2.8 | NC |
| | repressor CREM | | | |
| m89646_at | ribosomal protein | 9.7 | NC | NC |
| | S24 RNA | | | |
| d10938exon_s | BDNF | 9.6 | NC | NC |
| K02814_g_at | ribosomal protein | 9.5 | NC | NC |
| | S24 RNA | | | |
| x13044_at | MHC-associated | 9.4 | 9 | NC |
| | invariant chain | | | |
| | gamma | | | |
| rc_ai639441 | EST | 9.3 | NC | NC |
| U23146cds_s_ | mitogenic | 9.3 | NC | NC |
| | regulation | | | |
| | SSECKS (322) | | | |
| | gene | | | |
| u53505_s_at | type II | 9.3 | NC | NC |
| | iodothyronine | | | |
| | deiodinase RNA | | | |
| L12025_at | tumor-associated | 9.2 | 3.8 | NC |
| | blycoprotein E4 | | | |
| | (Tage4) RNA | | | |
| rc_aa800797 | EST | 9 | NC | NC |
| M11596_at | Rat beta-type | | | |
| | calcitonin gene- | | | |
| | related peptide | | | |
| | RNA | | | |
| m58364_at | Rat GTP | 9 | NC | NC |
| | cyclohydrolase I | | | |
| | RNA | | | |
| x14319cds_g | T-cell receptor | 8.9 | NC | NC |
| | beta chain | | | |
| U41453_at | PKC binding | 8.9 | NC | NC |
| | protein and | | | |
| | substrate RNA | | | |
| rc_aa799729 | EST | 8.8 | 2.2 | NC |
| af083418 | insulin receptor | | | NC |
| | substrate-2 | | | |
| | (IRS-2) RNA | | | |
| rc_aa875099 | EST | 8.8 | 8.7 | NC |
| af082124_s_a | aryl hydrocarbon | 8.7 | 10 | NC |
| | receptor (AHR) | | | |
| | RNA | | | |
| aj01116_at | endothelial nitric | 8.7 | 2.1 | NC |
| | oxide synthase | | | |
| x06769cds_at | c-fos | 8.6 | NC | NC |
| rc_aa799450 | EST | 8.5 | 4 | NC |
| S56464RNA_a | HKII = | 8.4 | NC | NC |
| | hexokinase II | | | |
| ab006710_s_a | 6-phosphofructo- | 8.3 | 10.4 | NC |
| | 2-kinase/fructose- | | | |
| | 2,6- | | | |
| | bisphosphatase | | | |
| rc_aa858607 | EST | 8.3 | NC | NC |
| rc_ai176856 | EST | 8.2 | 4.3 | NC |
| aj004858_at | Sry-related | 8.2 | NC | NC |
| | HMG-box protein | | | |
| | Sox 11 | | | |
| x67108_at | brain and all other | 8.1 | NC | NC |
| | organ-derived | | | |
| | neurotrophic | | | |
| | factor (exon IV) | | | |
| Y00396RNA_g | c-myc | 8.1 | NC | NC |
| rc_aa800784 | EST | 8 | NC | NC |
| rc_ai071531 | EST | 7.9 | 3.5 | NC |
| rc_ai012030 | EST | 7.7 | 5 | NC |
| rc_aa894338 | EST | 7.6 | 5.7 | NC |
| rc_aa875126 | EST | 7.6 | 8 | NC |
| L33869_at | ceruloplasmin | 7.6 | 3.2 | NC |
| | RNA | | | |
| rc_aa859827 | | 7.6 | 15.9 | NC |
| AF081503 | inhibitor of | 7.5 | NC | NC |
| | apoptosis protein | | | |
| | (rIAP) | | | |
| U15550 | tenascin-C RNA | 7.2 | 3.6 | NC |
| U09401-s_at | tenascin RNA | 7.1 | 5.5 | NC |
| s67722_s_at | cyclooxygenase | 7 | 2.2 | NC |
| | isoform COX-2 | | | |
| s61865_s_at | syndecan = | 7 | 3.3 | NC |
| | heparan sulfate | | | |
| | proteoglycan core | | | |
| | protein | | | |
| rc_ai619318 | EST | 7 | NC | NC |
| rc_ai045858 | EST | 6.9 | 6.2 | NC |
| d30649RNA_s | phosphodiesterase | 6.9 | 6.1 | NC |
| | 1 | | | |
| L25925_s_at | cyclooxygenase-2 | 6.7 | 2.1 | NC |
| | RNA | | | |
| U96490_at | Rattus norvegicus | 6.7 | NC | NC |
| | liver RNA | | | |
| rc_aa875131 | | 6.7 | NC | NC |
| Af030091UTR#1 | cyclin ania-6a | 6.6 | NC | NC |
| | RNA | | | |
| j05132_s_at | Rat 3-methyl- | 6.6 | 9.3 | NC |
| | cholanthrene- | | | |
| | inducible | | | |
| | truncated UDP | | | |
| D14869_s_at | prostaglandin E2 | 6.5 | NC | NC |
| | receptor EP3 | | | |
| | subtype (rEP3) | | | |
| rc_aa891901_ | EST | 6.5 | NC | NC |
| M63101cds_at | Rat interleukin 1 | 6.3 | NC | NC |
| | receptor | | | |
| | antagonist gene | | | |
| J05122_at | peripheral-type | 6.3 | 6 | NC |
| | benzodiazepine | | | |
| | receptor | | | |
| x60769RNA_s | silencer factor B | 6.3 | 2.4 | NC |
| x96437RNA_g | PRG1 gene | 6.2 | 2.1 | NC |
| x07285cds_s | basic fibroblast | 6.2 | 7.1 | NC |
| | growth factor | | | |
| x06769cds_g | c-fos | 6.2 | NC | NC |
| L27060_at | phosphodiesterase | 6.1 | NC | NC |
| | RNA | | | |
| AJ002949cds | retinoic acid | 5.9 | | NC |
| | receptor alpha 1 | | | |
| L32591RNA_a | GADD45 RNA | 5.9 | 3.9 | NC |
| D84418_s_at | chromosomal | 5.9 | 4.5 | NC |
| | protein HMG2 | | | |
| rc_aa892553 | EST | 5.8 | 7.9 | NC |
| k02184_at | Rat major cute | 5.8 | 2.8 | NC |
| | phase alpha-1 | | | |
| | protein (MAP) | | | |
| rc_aa957003 | EST | 5.8 | NC | NC |
| M8310_g_at | SM22 RNA | 5.8 | NC | NC |
| L27059_s_at | phosphodiesterase | 5.7 | NC | NC |
| | RNA | | | |
| rc_ai639338 | EST | 5.6 | NC | NC |
| M34134_s_at | alpha- | 5.6 | NC | NC |
| | tropomyosin | | | |
| | (TMBr-2) RNA | | | |
| L20681_at | Rat proto- | 5.5 | NC | NC |
| | oncogene (Ets-1) | | | |
| | RNA | | | |
| x0651RNA-s | Rat RNA for | 5.5 | 4.6 | NC |
| | syndecan | | | |
| L14610_at | Rat transcription | 5.5 | NC | NC |
| | factor RZR-beta | | | |
| | gene | | | |
| rc_A1070295 | EST | 5.5 | 3.5 | NC |
| rc_ai030286 | EST | 5.4 | NC | NC |
| x61381cds_s | interferon induced | 5.4 | 5.1 | NC |
| | RNA | | | |
| M55017exon_s | Rat nucleolin | 5.4 | 6.5 | NC |
| | gene | | | |
| U62667_at | stannicalcin | 5.3 | NC | NC |
| | (rSTC) RNA | | | |
| rc_aa858586 | EST | 5.3 | NC | NC |
| rc_aa8800613 | EST | 5.3 | 2.4 | NC |
| u09540_g_at | cytochrome P450 | 5.3 | 3.6 | NC |
| | (CYP1B1) RNA | | | |
| u69884_at | calcium-activated | 5.3 | NC | NC |
| | potassium 0.3 | | | |
| | channel rSK3 | | | |
| | (SK) RNA | | | |
| M98820_at | Rat interleukin | 5.3 | NC | NC |
| | 1-beta RNA | | | |
| M15644_at | Rat OMP RNA | 5.2 | NC | NC |
| | encoding the | | | |
| | olfactory neuronal | | | |
| | specific protein | | | |
| U31599_g_at | MHC class II-like | 5.2 | 5.6 | NC |
| | beta chain | | | |
| | (RT1.DMb) RNA | | | |
| L13039_s_at | annexin II RNA | 5.2 | 2.3 | NC |
| x57523_g_at | mtp1RNA | 5.2 | 8.3 | NC |
| rc_aa859305 | | 5.2 | 5.3 | NC |
| d89070cds_s | non-inducible | 5.1 | 2.3 | NC |
| | carbonyl | | | |
| | reductase | | | |
| x63594cds_at | RL/IF-1 RNA | 5.1 | NC | NC |
| af008650_at | somatostatin | 5.1 | 3.5 | NC |
| | receptor-like | | | |
| | protein (SLC1) | | | |
| | RNA | | | |
| rc_aa817854 | EST | 5.1 | 5.9 | NC |
| d29766cds#1 | Crk-associated | 5 | 5.6 | NC |
| | substrate, p130 | | | |
| J03624_at | Rat galanin (a | 5 | 3 | NC |
| | neuropeptide) | | | |
| | RNA | | | |
| rc_aa800962 | EST | 5 | NC | NC |
| rc_aa799686 | EST | 5 | 6.3 | NC |
| M60616_at | Rat collagenase | 4.9 | NC | NC |
| | (UMRCase) RNA | | | |
| rc_A1014163 | EST | 4.9 | 2.3 | NC |
| x63594cds_g_ | RL/IF-1 RNA | 4.8 | NC | NC |
| ab005900_at | endothelial | 4.8 | NC | NC |
| | receptor for | | | |
| | oxidized low | | | |
| | density | | | |
| af036537 | homocysteine | 4.7 | NC | NC |
| | respondent | | | |
| | protein HCYP2 | | | |
| | RNA | | | |
| z22812_at | interleukin-1 | 4.7 | NC | NC |
| | receptor type 2 | | | |
| u04835_at | CREMdeltaC—G | 4.7 | 2.1 | NC |
| | gene | | | |
| U16674_at | interleukin- | 4.7 | NC | NC |
| | 12p40 RNA | | | |
| D29769_at | bone | 4.7 | NC | NC |
| | morphogenic | | | |
| | protein-7 | | | |
| x54686cds_at | pJunB gene | 4.6 | NC | NC |
| rc_ai639457 | EST | 4.6 | NC | NC |
| L46593cds_at | small proline-rich | 4.6 | 4 | NC |
| | protein (spr) gene | | | |
| af28784cds# | glial fibrillary | 4.6 | 6.2 | NC |
| | acidic proteins | | | |
| | alpha and delta | | | |
| | (GFAP) gene | | | |
| m80633_at | Rat adenylyl | 4.6 | 5.2 | NC |
| | cyclase type (IV) | | | |
| | RNA | | | |
| rc_aa799448 | EST | 4.6 | NC | NC |
| x60351cds_s | alpha B-crystallin | 4.5 | 2.4 | NC |
| s82649_s_at | Narp = neuronal | 4.5 | 2.2 | NC |
| | activity-regulated | | | |
| | pentraxin | | | |
| u78102_at | krox20 RNA | 4.5 | NC | NC |
| rc_aa926129_ | EST | 4.5 | 4.9 | NC |
| x98377_at | RNA for emerin | 4.5 | NC | NC |
| rc_ai639233 | EST | 4.4 | NC | NC |
| x95986RNA#1 | CBR gene | 4.4 | NC | NC |
| af087944RNA | monocyte | 4.4 | 2.6 | NC |
| | differentiation | | | |
| | antigen CD14 | | | |
| | gene | | | |
| RC_AA891041 | EST | 4.3 | NC | NC |
| j04563_at | Rat cAMP | 4.3 | 5.4 | NC |
| | phosphodiesterase | | | |
| | RNA | | | |
| rc_ai233219 | EST | 4.3 | NC | NC |
| u33500_g_at | retinol | 4.3 | NC | NC |
| | dehydrogenase | | | |
| | type II RNA | | | |
| rc_ai169756 | EST | 4.3 | 1.7 | NC |
| rc_aa900476 | EST | 4.2 | NC | NC |
| L32591RNA_g | GADD45 RNA | 4.2 | 3 | NC |
| rc_aa875126 | EST | 4.2 | 8.7 | NC |
| L20913_s_at | vascular | 4.2 | NC | NC |
| | endothelial | | | |
| | growth factor | | | |
| | form 3 RNA | | | |
| x71127_s_at | complement | 4.1 | 3.6 | NC |
| | protein C1q beta | | | |
| | chain | | | |
| af083269_at | p41-Arc RNA | 4.1 | 3.3 | NC |
| rc_aa799773 | EST | 4.1 | 4.7 | NC |
| rc_ai639402 | EST | 4.1 | NC | NC |
| a30543cds_s_ | p-Meta-a RNA | 4.1 | 4.4 | NC |
| | for CD44 surface | | | |
| | protein from | | | |
| | patent | | | |
| | WO9117248 | | | |
| aj222813_s_a | precursor | 4.1 | 3.8 | NC |
| | interleukin 18 | | | |
| | (IL-18) | | | |
| rc_ai6i39302 | EST | 4 | NC | NC |
| rc_ai639161 | EST | 4 | 7.5 | NC |
| rc_aa946044 | EST | 3.9 | 2.8 | NC |
| m19257_at | Rat cysosolic | 3.9 | 4 | NC |
| | retinol-binding | | | |
| | protein (CRBP) | | | |
| | RNA | | | |
| Y10619cds_at | transcriptional | 3.8 | 3.5 | NC |
| | regulator, Relax | | | |
| x99121RNA#1 | RT6 gene, exon | 3.8 | NC | NC |
| | 2, testis | | | |
| x74565cds_g_ | TBFII RNA for | 3.8 | 5.4 | NC |
| | polypyrimidine | | | |
| | tract binding | | | |
| d17370_g_at | cystathionine | 3.8 | 3.8 | NC |
| | gamma-lyase | | | |
| af086624_s_a | serine threonine | 3.8 | NC | NC |
| | kinase (pim-3) | | | |
| | RNA | | | |
| m13979_at | Rat brain and all | 3.8 | 2.1 | NC |
| | other organ | | | |
| | glucose- | | | |
| | transporter | | | |
| | protein RNA | | | |
| U13396_at | protein-tyrosine | 3.7 | NC | NC |
| | kinase (JAK2) | | | |
| | RNA | | | |
| d00913_g_at | intercellular | 3.7 | NC | NC |
| | adhesion | | | |
| | molecule-1 | | | |
| rc_aa799323_{—} | EST | 3.7 | NC | NC |
| d90404_at | cathepsin C | 3.7 | 2.8 | NC |
| d89069_f_at | inducible | 3.7 | NC | NC |
| | carbonyl | | | |
| | reductase | | | |
| af053362_at | Rattus norvegicus | 3.7 | NC | NC |
| | death effector | | | |
| | domain- | | | |
| | containing protein | | | |
| | DEFT RNA | | | |
| m60753_s_at | catechol-O- | 3.7 | 4.5 | NC |
| | methyltransferase | | | |
| | RNA | | | |
| rc_aa891576 | EST | 3.6 | NC | NC |
| m18330_at | Rat protein kinase | 3.6 | 3.3 | NC |
| | C delta | | | |
| | subspecies | | | |
| m32062_at | Rat Fc-gamma | 3.6 | 1.5 | NC |
| | receptor RNA | | | |
| rc_aa866443 | EST | 3.6 | NC | NC |
| d90404_g_at | cathepsin C | 3.6 | NC | NC |
| U099870_at | Vmajor vault | 3.6 | 2.5 | NC |
| | protein RNA | | | |
| x62951RNA_s | R. norvegicus | 3.5 | NC | NC |
| | RNA (pBUS19) | | | |
| | with repetitive | | | |
| af00898_at | p58/p45 RNA, | 3.5 | 3.3 | NC |
| | alternatively | | | |
| | spliced form | | | |
| | clone H | | | |
| m34253_g_at | Rat-interferon | 3.5 | 3 | NC |
| | regulatory factor1 | | | |
| | (IRF-a) RNA | | | |
| x63434_at | R. norvegicus | 3.5 | NC | NC |
| | RNA for | | | |
| | urokinase-type | | | |
| | plasminogen | | | |
| | activator | | | |
| rc_ai71962 | EST | 3.5 | 1.9 | NC |
| rc_aa892775 | EST | 3.4 | 2.3 | NC |
| af074608RNA | MHC class I | 3.4 | 3 | NC |
| | antigen | | | |
| | (RT1.EC2) gene | | | |
| rc_ai171966 | EST | 3.4 | 3.8 | NC |
| j04792_at | ornithine | 3.4 | 1.7 | NC |
| | decarboxylase | | | |
| | ODC) gene | | | |
| d8557s_at | RYB-a | 3.5 | 3.5 | NC |
| rc_ai638945 | EST | 3.4 | NC | NC |
| rc_aa892851 | EST | 3.4 | NC | NC |
| rc_aa875032 | EST | 3.4 | NC | NC |
| af083269_g_at | p41-Arc RNA | 3.4 | 3.3 | NC |
| af092090_at | cp151 RNA | 3.4 | 2.7 | NC |
| m63122_at | Rat tumor | 3.4 | 2.8 | NC |
| | necrosis factor | | | |
| | receptor (TNF | | | |
| | receptor) | | | |
| af036537_at | homocysteine | 3.4 | NC | NC |
| | respondent | | | |
| | protein | | | |
| x71127_at | complement | 3.3 | 2.7 | NC |
| | protein C1q beta | | | |
| rc_ai639372_ | EST | 3.3 | NC | NC |
| u05014_at | Rattus norvegicus | 3.3 | 3.9 | NC |
| | Sprague/Dawley | | | |
| | PHAS-a | | | |
| u23407_at | Rattus norvegicus | 3.3 | 9.2 | NC |
| | cellular retinoic | | | |
| | acid-binding | | | |
| | protein II | | | |
| | (CRABP II) RNA | | | |
| M63282_at | Rat leucine zipper | 3.3 | NC | NC |
| | protein RNA | | | |
| U88572_at | AMPA receptor | 3.3 | 3.5 | NC |
| | interacting protein | | | |
| | GRIP RNA | | | |
| j00780_at | rat preprorelaxin | 3.3 | NC | NC |
| | RNA | | | |
| rc_ai639042 | | 3.3 | NC | NC |
| U77829RNA_s | Rattus norvegicus | 3.3 | 3.4 | NC |
| | gas-5 growth | | | |
| | arrest homolog | | | |
| | NCn-translated | | | |
| | RNA sequence | | | |
| s77494_s_at | lysyl oxidase | 3.3 | NC | NC |
| | {3Nuntranslated | | | |
| | region} [rats, | | | |
| | aorta smooth | | | |
| | muscle cell | | | |
| rc_ai176456 | EST | 3.3 | 2.3 | NC |
| rc_aa892750 | EST | 3.2 | NC | NC |
| M55534RNA_s | Rat alpha- | 3.2 | 2 | NC |
| | crystallin b chain | | | |
| | RNA | | | |
| af030089UTR# | Rattus norvegicus | 3.2 | 4 | NC |
| | activity and | | | |
| | neurotransmitter- | | | |
| | induced early | | | |
| | gene | | | |
| rc_aa800701 | EST | 3.2 | NC | NC |
| rc_aa945737 | EST | 3.2 | NC | NC |
| rc_ai070295 | EST | 3.2 | 1.9 | NC |
| M90661_at | Rattus norvegicus | 3.2 | NC | NC |
| | insulin receptor- | | | |
| | related receptor- | | | |
| | alpha subunit | | | |
| | RNA | | | |
| U49930_g_at | ICE-like cysteine | 3.2 | 2.7 | NC |
| | protease (Lice) | | | |
| | RNA | | | |
| M92433exon#1 | Rattus norvegicus | 3.1 | NC | NC |
| | nerve growth | | | |
| | factor-induced | | | |
| | clone C (NGFI-C) | | | |
| | gene | | | |
| rc_ai639149 | EST | 3.1 | NC | NC |
| rc_aa859740 | EST | 3.1 | NC | NC |
| D10729_s_at | Rat RNA for | 3.1 | 4.9 | NC |
| | proteasome | | | |
| | subunit RC | | | |
| x91810_at | R. norvegicus | 3.1 | NC | NC |
| | RNA for Stat3 | | | |
| | protein | | | |
| x62952 | R. norvegicus | 3.1 | 3.2 | |
| | RNA for vimentin | | | |
| rc_ai178267 | EST | 3.1 | NC | NC |
| af020618_gc_ a | Rattus norvegicus | | | |
| | progression | | | |
| | elevated gene 3 | | | |
| | protein RNA | | | |
| d00575_at_ | Rattus norvegicus | 3.1 | NC | NC |
| | RNA for pituitary | | | |
| | glycoprotein | | | |
| | hormone alpha- | | | |
| | subunit precursor, | | | |
| | complete cds | | | |
| rc_aa892578_ | EST | 3.1 | 2.7 | no |

NC = No Change. In the above table there were no changes of the above genes with hypoxia.

TABLE 6

| | | | Fold |
|---|---|---|---|
| Probe Set | Name | Induction | Change |
| rc_AA799861_g_at | interferon regulatory | I | 32.9 |
| | factor 7 | | |
| U42719_at | C4 complement protein | I | 20.8 |
| M64791_at | salivary proline-rich protein | I | 7.2 |
| | (RP4) gene | | |
| rc_AA799861_at | interferon regulatory | I | 7.1 |
| | factor 7 | | |
| rc_A1045858_at | FK506 binding protein 1a | I | 6.7 |
| rc_AA946503_at | alpha 2 mu globulin-related | I | 5.7 |
| | protein | | |
| rc_AI172247_at | xanthine dehydrogenase | I | 5.7 |
| rc_AA926129_at | Sacm21/RT1-A intergenic | I | 5 |
| | region, partial RT1-A gene | | |
| | for MHC class I ant | | |
| U80915_s_at | EAAT4 Na+-dependent | I | 4.9 |
| | glutamate transporter | | |
| rc_AA893822_at | C3H DNA damage repair | I | 4.7 |
| | and recombination protein | | |
| | RAD52 | | |
| rc_AA639161_at | asparaginyl-tRNA | I | 4.5 |
| | synthetase | | |
| M83107_g_at | SM22 RNA | I | 4.5 |
| x07285cds_s_at | basic fibroblast growth | I | 4 |
| | factor | | |
| x97754 | 17-beta-hydroxysteroid | I | 3.9 |
| | dehydrogenase type 1 | | |
| rc_AI638951_at | DCoH gene; pterin-4a- | I | 3.6 |
| | carbinolamin dehydratase | | |
| rc_AI639173_at | Homo sapiens genomic | I | 3 |
| | DNA, chromosome 8p11.2 | | |
| rc_AI639088_at | Mus musculus clone | I | 2.9 |
| | UWGC: mbac82 from | | |
| | 14D1-D2 | | |
| rc_AI639528_at | KIAA0772 gene product | I | 2.9 |
| rc_AA894226_g_at | Cpn 10-rs5 pseudogene | I | 2.8 |
| x61381cds_s_at | interferon induced RNA | I | 2.8 |
| x13905cds_at | ras-related rab1B protein | I | 2.8 |
| rc_AA946044_s_at | Lyn B tyrosine kinase | I | 2.7 |
| M62889_s_at | sucrase-isomaltase | I | 2.5 |
| M95780_at | G protein gamma-5 subunit | I | 2.5 |
| | RNA | | |
| rc_AI177256_at | Human DNA sequence | I | 2.5 |
| | from clone GS1-aa5M3 on | | |
| | chromosome Xq171-2 | | |
| x06801cds_I_at | vascular smooth muscle | I | 2.4 |
| | alpha-actin | | |
| rc_AA892564_at | 6-pyruvoyl-tetrahydro- | I | 2.4 |
| | protein synthase | | |
| Y07704_g_at | Best5 protein | I | 2.4 |
| U83119_f_at | retrotransposon ORF2 RNA | I | 2.4 |
| rc_AA894016_at | Human DNA sequence | I | 2.3 |
| | from clone RP11-353c18 | | |
| | on chromosome 20 | | |
| rc_AA892895_I_at | ribosomal protein S15 | I | 2.3 |
| rc_Aa893242_g_at | long-chain acyl-CoA | I | 2.3 |
| | synthetase | | |
| rc_AI639410_I_at | Pneumocystic carinii f. sp. | I | 2.2 |
| | carinii Cdc2 cyclin- | | |
| | dependent kinase | | |
| x53581cds#5_f_at | long interspersed repetitive | I | 2.2 |
| | DNA containing 7 ORF's | | |
| rc_AI639447_at | TANK binding kinase | I | 2.1 |
| | TBK1 | | |
| rc_AA859740_at | hepara sulfate 6-0-sulfo- | I | 2.1 |
| | transferase 1 (Hs6Stl). | | |
| | RNA | | |
| M58040_at | Rat transferring receptor | I | 2.1 |
| | RNA | | |
| rc_AI639410_s_at | Pneumocystis carinii f. sp. | I | 2 |
| | carinii Cdc2 cyclin- | | |
| | dependent kinase | | |
| M13101cds_f_at | Rat long interspersed | I | 2 |
| | repetitive DNA sequence | | |
| | LINE4 (L1Rn) | | |
| x07686cds_s_at | Rat L1Rn B6 repetitive | I | 2 |
| | DNA element | | |
| rc_AI012030_at | Rattus norvegicus Matrix | I | 1.9 |
| | Gla protein (Mgp), RNA | | |
| rc_AI012534_at | Rattus norvegicus TFIIA | I | 1.9 |
| | small subunit RNA | | |
| rc_AA893871_at | Homo sapiens 12p12 BAC | I | 1.9 |
| | RPCI11-1018J8 | | |
| x05472cds#3_f_at | Rat 2.4 kb repeat DNA | I | 1.9 |
| | right terminal region | | |
| M13100cds#6_f_at | Rat long interspersed | I | 1.9 |
| | repetitive DNA sequence | | |
| | LINE3 (L1Rn) | | |
| AF028784cds#1_s_at | Rattus norvegicus glial | I | 1.8 |
| | fibrillary acidic proteins | | |
| | alpha and delta (GFAP) | | |
| | gene | | |
| L06040_s_at | Rattus norvegicus | MI | 7.7 |
| | 12-lipoxygenase RNA | | |
| M649_f_at | Rattus norvegicus | MI | 7.2 |
| | 12-lipoxygenase RNA | | |
| rc_AA891717_g_at | transcription factor; USF 1 | MI | 6.9 |
| | gene; USF1 protein | | |
| rc_aa858586_at | chromatin structural protein | MI | 6.4 |
| | homolog Supt5hp (Supt5h) | | |
| D10729_s_at | Rat RNA for proteasome | MI | 6.3 |
| | subunit RC1 | | |
| z46614cds_at | R. norvegicus RNA for | MI | 5.7 |
| | caveolin | | |
| rc_AI639498_I_at | Drosophila melanogaster | MI | 5.4 |
| | genomic scaffold | | |
| rc_AA859966 | inositol 1,4,5-triphosphate | MI | 5.2 |
| | receptor type I RNA | | |
| rc_AA893781 | Homo sapiens KIAA0050 | MI | 4 |
| | gene product | | |
| rc_AA892553 | Rattus norvegicus signal | MI | 4 |
| | transducer and activator of | | |
| | transcription 1 (Stat1) RNA | | |
| rc_AI639512 | surfactant protein A (SP-A) | MI | 4 |
| L23077_at | zinc finger protein | MI | 3.8 |
| rc_AI639170 | Homo sapiens RNA | MI | 3 |
| | helicase-related protein | | |
| | RNA | | |
| L00382cds_at | Rat skeletal muscle beta- | MI | 2.9 |
| | tropomyosin and fibroblast | | |
| | tropomyosin 1 gene | | |
| rc_AI639339_at | Arabidopsis thaliana | MI | 2.8 |
| | chromosome 1 BAC F5D21 | | |
| | genomic sequence | | |
| rc_AA891944 | interferon-g induced | MI | 2.7 |
| | GTPase | | |
| rc_AI639372 | Homo sapiens KIAA0854 | MI | 2.7 |
| | protein (KIAA05854) | | |
| x16262_s_at | Rat RNA for alternatively | MI | 2.6 |
| | spliced smooth muscle | | |
| | myosin heavy chain | | |
| AF102853 | Rattus norvegicus | MI | 2.5 |
| | membrane-associated | | |
| | guanylate kinase-interacting | | |
| | protein 1 Maguin-1 RNA | | |
| AJ224680 | Rattus norvegicus RNA | MI | 2.4 |
| | for glutamic-acid rich | | |
| | protein | | |
| J05132_s_at | Rat 3-methylcholanthrene- | MI | 2.3 |
| | inducible truncated UDP- | | |
| rc_AI639342_at | Homo sapiens PAC clone | MI | 2 |
| | RP4-687K1 | | |
| x52711 | Rat RNA for Mx1 protein | MI | 2 |
| E12286cds_at | cDNA encoding rat GM2 | MI | 2 |
| | activator protein | | |
| rc_AA875646 | Homo sapiens clone 25076 | D | 13.7 |
| | RNA sequence | | |
| M93257_s_at | Rattus norvegicus | D | 13 |
| | cathechol-O-methyl- | | |
| | transferase RNA | | |
| U50412_at | phosphoinositide 3-dinase | D | 10.7 |
| | regulatory subunit p85alpha | | |
| | RNA | | |
| AI007530_f_at | Homo sapiens NADH: | D | 10.6 |
| | ubiquinone oxidoreductase | | |
| | MLRQ subunit | | |
| rc_AA924925_at | Dri 42 gene; ER- | D | 9.2 |
| | transmembrane protein | | |
| L81138exon_I_at | Rps2rgene | D | 6.1 |
| D64045_s_at | phosphatidylinositol | D | 5.2 |
| | 3-kinase p85 alpha subunit | | |
| Y08139_at | dermo-1 protein; | D | 5.2 |
| | helix-loop-helix protein | | |
| | (vascular smooth muscle) | | |
| rc_AA818122_f_at | hydroxysteroid | D | 5 |
| | sulfotransferase subunit | | |
| rc_AA818593 | phosphatidate | D | 4.7 |
| | phosphohydrolase type 2 | | |
| | RNA | | |
| rc_AA799480_at | R. norvegicus RNA | D | 4.2 |
| | (pJG116) with repetitive | | |
| | elements | | |
| AF050661UTR#1_at | activity and | D | 3.9 |
| | neurotransmitter-induced | | |
| | early gene 9 (ania-9) RNA | | |
| rc_AI178971_at | GLUTAMINE | D | 3.8 |
| | SYNTHETASE | | |
| L26292_g_at | FSH-regulated protein RNA | D | 3.7 |
| S62933_I_at | receptor tyrosine kinase | D | 3.5 |
| | (TrkC(ki14)) RNA | | |
| X00975_g_at | Rat MLC2 gene for muscle | D | 3.4 |
| | myosin light chain 2 | | |
| D82071_at | hematopoietic prostaglandin | D | 3.2 |
| | D synthase | | |
| X64563cds_at | plasminogen activator | D | 3.1 |
| | inhibitor 2 type A (PAI2A) | | |
| U78102_at | krox20 RNA | D | 2.8 |
| rc_H31411_at | Mus musculus chromosome | D | 2.7 |
| | 18 clone | | |
| U19866 | growth factor (Arc) RNA | D | 2.6 |
| M84149_at | Rat IgH chain VJ region | D | 2.5 |
| | RNA | | |
| AF075382_at | suppressor of cytokine | D | 2.4 |
| | signaling-2 (SOCS-2) RNA | | |
| U17254 | immediate early gene | D | 2.2 |
| | transcription factor NGFI-B | | |
| | RNA | | |
| U17254_g_at | immediate early gene | D | 2.2 |
| | transcription factor NGFI-B | | |
| | RNA | | |
| X60660RNA_g_at | Novel genes for potential | D | 2.1 |
| | ligand-binding proteins in | | |
| | subregions of 3CH134/ | | |
| | CL100 | | |
| S81478_s_at | PTPase = oxidative stress- | D | 2 |
| | inducible protein tyrosine | | |
| | phosphatase | | |
| S77492_I_at | bone morphogenetic | D | 1.9 |
| | protein-3 | | |
| X06769cds_at | C-fos | MD | 8.4 |
| D63860_s_at | prepro bone morphogenetic | MD | 3.8 |
| | protein-3 | | |
| rc_AA859552 | skeletal muscle elongation | MD | 3.4 |
| | factor-2 kinase | | |
| D26307cds_at | Rattus norvegicus jun-D | MD | 2.8 |
| | gene | | |
| rc_AA891041_at | | MD | 2.3 |
| S74351_s_at | protein tyrosine | MD | 2.1 |
| | phosphatase | | |

This list of hypoxia-regulated genes includes those that increased (I), had a marginal increase (MI) as judged statistically, a decrease (D), or a marginal decrease (MD) as judged statistically. It should be emphasized that the pattern of expression in the blood, brain, and all other organ samples include increased as well as decreased genes or proteins in the injury banks that are formed.

### Example 3

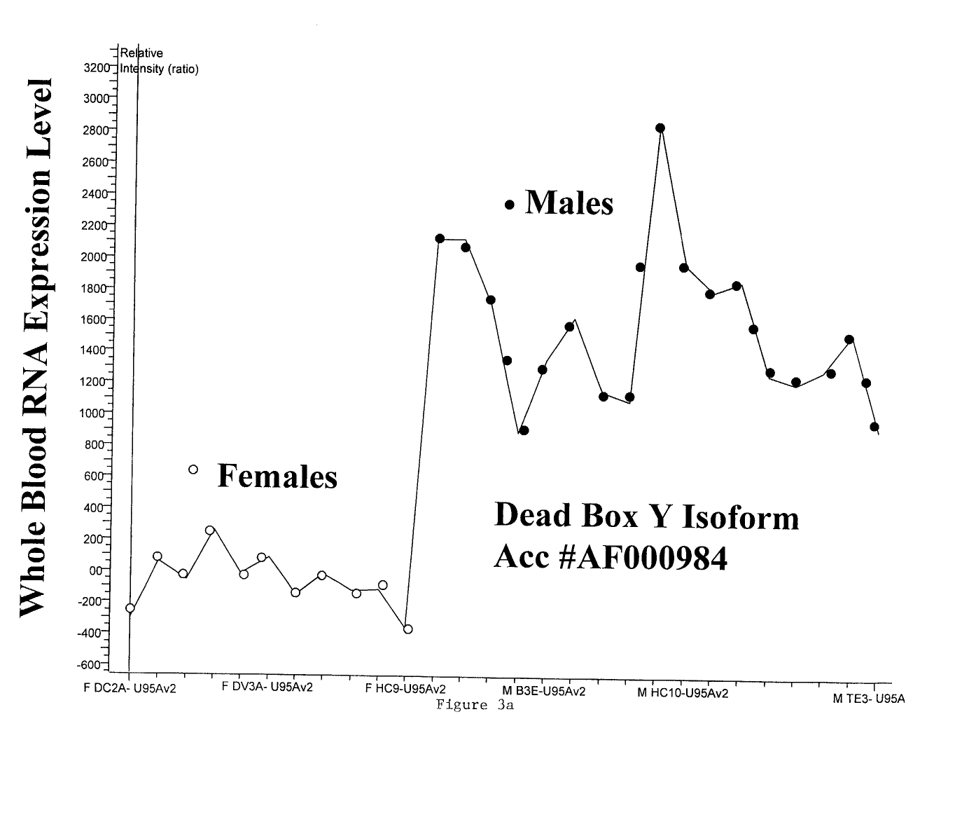

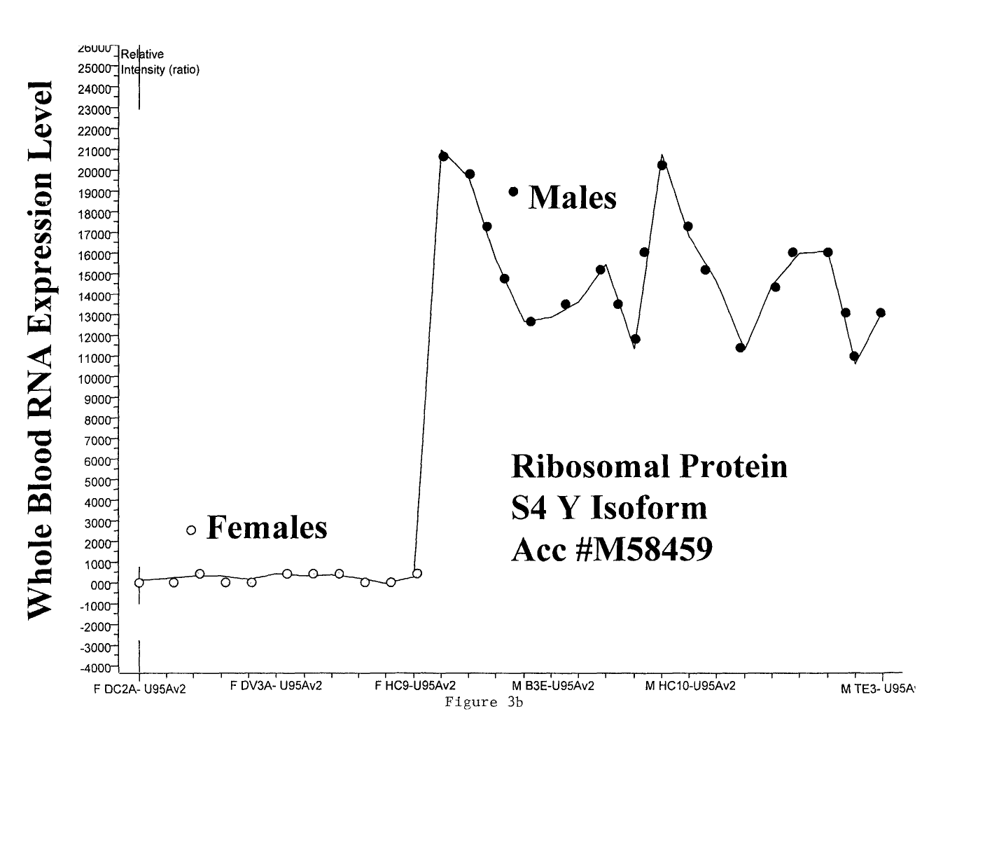

This example demonstrates the ability to differentiate between male and female blood samples based on patterns of expression. Blood from over 30 patients is collected from healthy controls as well as from patients with various neurological problems, including headaches, seizures, idiopathic Parkinson's disease, progressive supranuclear palsy, and psychosis. The blood cells are isolated, the RNA extracted, and then processed on commercially available chips (human Affymetrix chips). The RNA is analyzed using the statistical program called SAM (Significance Analysis of Microarrays) to determine the genes expressed more significantly in males as compared to females. As shown in FIG. 3a and 3b, over 20 genes are highly expressed in the blood samples of males as compared to females. The ticks on the X-axis represent individual patients, the first 11 being females and the next 21 representing males. The Y axis shows the expression of a single gene, Dead Box Y Isoform gene and Ribosomal Protein S4 Y Isoform, respectively. This graph shows that these genes are highly expressed in the blood cells of male patients and are expressed at very low levels in the blood of females.

Tables 7a and 7b below demonstrates the pattern of expression, of the upregulated genes, for males and females respectively. This data demonstrates how the pattern of expression in the blood of individuals is unique and can be used to predict the sex of an individual.

TABLE 7a
Upregulated genes in females

| Genbank | Description |
|---|---|
| X56199 | Human XIST, coding sequence a mRNA (locus DXS399E) |
| U76388 | Human steroidogenic factor 1 mRNA, complete cds |
| D10040 | Homo sapiens mRNA for long-chain acyl-CoA synthetase, |
| | complete cds |
| X78710 | H. sapiens MTF-1 mRNA for metal-regulatory transcription |
| | factor |
| U09564 | U09564/FEATURE = /DEFINITION = HSU09564 Human |
| | serine kinase mRNA, complete cds |
| U12569 | Human mu opioid receptor variant (MOR1) mRNA, |
| | complete cds |
| AF017257 | Homo sapiens chromosome 21 derived BAC containing |
| | erythroblastosis virus oncogene homolog 2 protein (ets-2) |
| | gene, complete cds |
| M20681 | Human glucose transporter-like protein-III (GLUT3), |
| | complete cds |
| AA135683 | zl10c08.r1 Homo sapiens cDNA, 5 end |
| AB002315 | Human mRNA for KIAA0317 gene, complete cds |
| U09877 | Human helicase-like protein (HLP) mRNA, complete cds |
| U45976 | Human clathrin assembly protein lymphoid myeloid |
| | leukemia (CALM) mRNA, complete cds |
| AL031775 | dJ30M3.3 (novel protein similar to C. elegans Y63D3A.4) |
| AA705628 | zf40a01.s1 Homo sapiens cDNA, 3 end |
| W26226 | 22e3 Homo sapiens cDNA |
| U70451 | Human myleoid differentiation primary response protein |
| | MyD88 mRNA, complete cds |
| U27467 | U27467/FEATURE = /DEFINITION = HSU27467 Human |
| | Bcl-2 related (Bfl-1) mRNA, complete cds |
| Y10745 | H. sapiens mRNA for inwardly rectifing potassium channel |
| | Kir4.2 |
| M83667 | M83667/FEATURE = mRNA/DEFINITION = |
| | HUMNFIL6BA Human NF-IL6-beta protein mRNA, |
| | complete cds |
| S82470 | S82470/FEATURE = /DEFINITION = S82470 BB1 = |
| | malignant cell expression-enhanced gene/tumor |
| | progression-enhanced gene [human, UM-UC-9 bladder |
| | carcinoma cell line, mRNA, 1897 nt] |
| AI341565 | qq94g11.x1 Homo sapiens cDNA, 3 end |
| M79321 | M79321/FEATURE = /DEFINITION = HUMLYNTK |
| | Human Lyn B protein mRNA, complete cds |
| M31932 | Human IgG low affinity Fc fragment receptor (FcRIIa) |
| | mRNA, complete cds |
| U31383 | Human G protein gamma-10 subunit mRNA, complete cds |
| AB011094 | Homo sapiens mRNA for KIAA0522 protein, partial cds |
| X95735 | Homo sapiens mRNA for zyxin |
| X52015 | H. sapiens mRNA for interleukin-1 receptor antagonist |
| D82351 | Human retropseudogene MSSP-1 DNA, complete cds |
| W28743 | 51a9 Homo sapiens cDNA |
| U43774 | Human Fc alpha receptor, splice variant FcalphaR a.2 |
| | (CD89) mRNA, complete cds |
| U00115 | Human zinc-finger protein (bcl-6) mRNA, complete cds |
| H15814 | yl28b07.s1 Homo sapiens cDNA, 3 end |
| AL049923 | Homo sapiens mRNA; cDNA DKFZp547E2210 (from |
| | clone DKFZp547E2210) |
| AB002344 | Human mRNA for KIAA0346 gene, partial cds |
| U02020 | Human pre-B cell enhancing factor (PBEF) mRNA, |
| | complete cds |
| D89974 | Homo sapiens mRNA for glycosylphosphatidyl inositol- |
| | anchored protein GPI-80, complete cds |
| AI984234 | wz57e04.x1 Homo sapiens cDNA, 3 end |
| X77094 | H. sapiens mRNA for p40phox |
| J05272 | Human IMP dehydrogenase type 1 mRNA complete cds |
| L18960 | Human protein synthesis factor (eIF-4C) mRNA, |
| | complete cds |
| AL008637 | Human DNA sequence from clone 833B7 on chromosome |
| | 22q12.3-13.2 Contains genes for NCF4 (P40PHOX) |
| | protein, cytokine receptor common beta chain precursor |
| | CSF2RB (partial), ESTs, CA repeat, STS, GSS |
| X59739 | Human ZFX mRNA for put. transcription activator, |
| | isoform 2 |
| U32315 | Human syntaxin 3 mRNA, complete cds |
| L78833 | L78833/FEATURE = cds#4/DEFINITION = HUMBRCA1 |
| | Human BRCA1, Rho7 and vatl genes, complete cds, and |
| | ipf35 gene, partial cds |
| AB011406 | Homo sapiens mRNA for alkalin phosphatase, complete cds |
| D14874 | Homo sapiens mRNA for adrenomedullin precursor, |
| | complete cds |
| AB018306 | Homo sapiens mRNA for KIAA0763 protein, complete cds |
| U24152 | U24152/FEATURE = /DEFINITION = HSU24152 Human |
| | p21-activated protein kinase (Pak1) gene, complete cds |
| U19775 | U19775/FEATURE = cds/DEFINITION = HSU19775 |
| | Human MAP kinase Mxi2 (MXI2) mRNA, complete cds |
| H04668 | yj49e08.r1 Homo sapiens cDNA, 5 end |
| AB007448 | Homo sapiens mRNA for OCTN1, complete cds |
| AL008637 | Human DNA sequence from clone 833B7 on chromosome |
| | 22q12.3-13.2 Contains genes for NCF4 (P40PHOX) |
| | protein, cytokine receptor common beta chain precursor |
| | CSF2RB (partial), ESTs, CA repeat, STS, GSS |
| M81637 | Human grancalcin mRNA, complete cds |
| L36069 | Human high conductance inward rectifier potassium |
| | channel alpha subunit mRNA, complete cds |
| L42243 | L42243/FEATURE = cds#3/DEFINITION = |
| | HUMIFNAM08 Homo sapiens (clone 51H8) alternatively |
| | spliced interferon receptor (IFNAR2) gene, exon 9 and |
| | complete cds s |
| J05008 | J05008/FEATURE = expanded_cds/DEFINITION = |
| | HUMEDN1B Homo sapiens endothelin-1 (EDN1) gene, |
| | complete cds |
| D38583 | Human mRNA for calgizzarin, complete cds |
| AF039656 | Homo sapiens neuronal tissue-enriched acidic protein |
| | (NAP-22) mRNA, complete cds |
| J05070 | Human type IV collagenase mRNA, complete cds |
| AF030339 | Homo sapiens receptor for viral semaphorin protein |
| | (VESPR) mRNA, complete cds |
| L18960 | L18960/FEATURE = /DEFINITION = HUMEIF4C Human |
| | protein synthesis factor (eIF-4C) mRNA, complete cds |
| AI885381 | wi93b01.x1 Homo sapiens cDNA, 3 end |

TABLE 7b
Upregulated genes in males

| Genbank | Description |
|---|---|
| M58459 | Human ribosomal protein (RPS4Y) isoform mRNA, complete |
| | cds |
| AF000984 | Homo sapiens dead box, Y isoform (DBY) mRNA, alternative |
| | transcript 2, complete cds |
| AF000986 | Homo sapiens Drosophila fat facets related Y protein |
| | (DFFRY) mRNA, complete cds |
| Y15801 | Homo sapiens mRNA for PRKY protein |
| U52191 | Human SMCY (H—Y) mRNA, complete cds |
| D86324 | Homo sapiens mRNA for CMP-N-acetylneuraminic acid |
| | hydroxylase, complete cds |
| AF000994 | Homo sapiens ubiquitous TPR motif, Y isoform (UTY) |
| | mRNA, alternative transcript 3, complete cds |
| Z98744 | histone H3.1 |
| AF000987 | Homo sapiens eIF-1A, Y isoform (EIF1AY) mRNA, complete |
| | cds |
| M30607 | Human zinc finger protein Y-linked (ZFY) mRNA, complete |
| | cds |
| AF055581 | Homo sapiens adaptor protein Lnk mRNA, complete cds |
| M60052 | Human histidine-rich calcium binding protein (HRC) mRNA, |
| | complete cds |

### Example 4

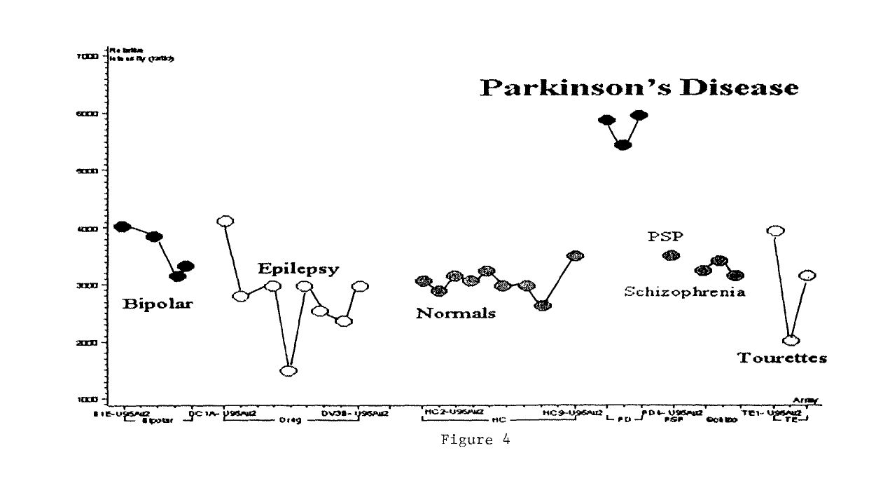

This example demonstrates the ability to assess Parkinson's disease based a sample's pattern of expression. To study the gene expression in Parkinson's patients, blood from over 30 patients is collected from healthy controls as well as from patients with a variety of disorders, including idiopathic Parkinson's patients with bradykinesia, rigidity and the characteristic tremor without dementia or evidence of any other neurological findings; progressive supranuclear palsy, bipolar disorder, schizophrenia, epilepsy, and Tourettes. A commercially available kit (Qiagen) is used to the blood cells from the whole blood samples, and total RNA isolated from the white blood cells. Two thirds of the RNA is used on DNA microarrays, and one third is used for PCR confirmation of the genes that are changed. After the purity of the RNA is checked (OD 280/OD 260=2), cDNA is synthesized from the total RNA and used to make biotin labeled cRNA. The cRNA is then applied to Affymetrix chips, human U95A chips that can screen for the expression of over 13,000 human genes including 11,000 known genes and 2,000 ESTs, and processed and scanned according to manufacturer's instructions. The chips are scanned twice for each patient sample. Genes that are expressed over two-fold compared to normals are plotted on figures. These genes are confirmed using standard techniques including PCR, Northern blotting or Western blotting. A separate statistical analysis is also applied to the data. The RNA is analyzed using the statistical program called SAM (Significance Analysis of Microarrays) to define the genes expressed more significantly in Parkinson's patients as compared to other patients. Once this analysis is performed, the data is used to perform a class prediction analysis. As shown in FIG. 4, genes SEQ ID NO:1 and SEQ ID NO:2 are expressed more highly in Parkinson's patients compared to other patients. The expression value of the genes is shown on the Y axis and the individual patients are plotted on the X-axis. The data demonstrates that the pattern of expression may be used to assess Parkinson's injury in an individual.

### Example 5

This example demonstrates the ability to assess stroke as compared to hemorrhage based on the pattern of expression for each injury. One 20 ml venous blood sample (in EDTA, two lavender top tubes) is obtained from patients at 24 hours (±4 hours) following: a large vessel ischemic stroke with a NIHSS of ≧10; following an intracerebral hemorrhage (ICH) with a NIHSS of ≧10; or following admission to the University of Cincinnati hospital for other neurological or medical reasons (controls). The blood cells are separated, followed by isolation of total RNA. Ischemic strokes and intracerebral hemorrhages are confirmed by clinical history, clinical neurological examinations, and CT or MRI scans performed within 72 hours.

The total RNA is used to synthesize cDNA and then biotin labeled cRNA. This is applied to human Affymetrix chips that are processed and scanned according to the manufacturer's instruction. Affymetrix Gene Chip software is used to determine which genes are scored as being present and absent and which genes show a two fold change following ischemic stroke compared to the controls and compared to the patients with intracerebral hemorrhages. The data is imported into Gene Spring, a commercially available biostatistic package, that allows for the calculation of fold changes of genes across all of the patients in all three groups, and for cluster analysis as shown in Example 1.

The primary analysis is Significance Analysis of Microarrays, which allows delineation all of the genes that are significantly expressed in ischemic stroke that are different from the genes expressed in the control group and in the intracerebral hemorrhage group, using a false discovery rate threshold of 5% or 10%. This defines a set of genes that are most reliably expressed following ischemia compared to the other samples. This set of genes is then used to define a prediction set of genes, S. The prediction set S of genes is then used to perform weighted voting on patient samples to determine if a patient sample conforms to the prediction set S or not. The first analysis is done to determine if the set S correctly predicts the initial set of ischemic samples used to derive the prediction set S. The second analysis determines if the set S correctly predicts a separate, new group of ischemic patient samples.

### Example 6

This example demonstrates assessment profusion state and/or excellent reperfusion, moderate reperfusion and/or poor reperfusion based on patterns of expression. All patients entered into the tPA/eptifibatide trial in Example 4 receive one of several tPA doses by 3 hours after an ischemic stroke. They also have a CT within the first 3 hours. At 24 hours following the stroke 20 cc of anticoagulated (EDTA) blood (two lavender tops) is obtained from patients with a NIHSS of ≧10, just as was done in Example 4. The blood cells are isolated, total RNA is purified, and then processed on human Affymetrix chips as described in Example 4. Using statistical methods defined in Example 4, patterns of expression characteristic of reperfusion as determined by MRA at 24 hours is determined. Also, patterns of expression that differentiates tPA treated patients without intracerebral hemorrhages, compared to those with tPA associated intracerebral hemorrhages, are determined. Lastly, a specific pattern of expression of patients with ischemic stroke treated with tPA as compared to patients with ischemic stroke not treated with tPA from Example 4 is determined.

All patents that receive tPA have a CT brain scan within 3 hours of the stroke, and have a MRI brain study one day later. The MRI evaluation includes a MRA (magnetic resonance angiogram), a diffusion MRI, and one MRI sequence to assess stroke volume (either a flash, T2, gradient echo or other sequence which will be standardized for all patients). MRA studies are evaluated by two independent neuroradiologists who rate the MRA at 24 hours as showing excellent, moderate or poor reperfusion. In addition, the MRA is evaluated using an MCID computer analysis system (SWANSON). An optical density threshold is set so that the vessels in the non-ischemic hemisphere are detected in the middle cerebral artery distribution which is defined using the same mask in every patient. The area occupied by these vessels is then computed automatically. Using a mirror image of the same region of the middle cerebral artery distribution in the ischemic hemisphere, the area occupied by the vessels is again computed automatically. Excellent reperfusion will be defined as the value in the ischemic hemisphere being >85% of the non-ischemic hemisphere. Poor reperfusion is defined as the value in the ischemic hemisphere being <45% of the non-ischemic hemisphere. Moderate reperfusion is defined as >45% and <85%. At least two MRA slices per patient are examined. Hence, there is a qualitative comparison of reperfusion performed, as well as a semi-quantitative comparison of reperfusion as determined by MRA. The pattern of expression of three groups of patients, excellent, moderate and poor reperfusion are then compared against each other to assess excellent reperfusion, moderate reperfusion or poor reperfusion. These patterns of expression may be used to assess reprofusion state and/or excellent, moderate and/or poor reperfusion of stroke in an individual.

### Example 7

The whole blood genomic responses of patients with status epilepticus, single seizures, or syncope are compared between the three conditions.

Adult males (n=10) and females (n=10) (all races between the ages of 18 and 75 years) with status epilepticus are entered into the example. Patients are considered if they (1) are diagnosed clinically as having had generalized status epilepticus and/or (2) have evidence of status epilepticus by EEG criteria. Clinical evidence of status epilepticus includes either continuous generalized seizures for 30 minutes, or intermittent generalized seizures for 30 minutes during which the patient does not fully recover consciousness. Within 18 to 28 hours of the start of the episode of status epilepticus, a single venous 12 ml blood sample (sterile in EDTA) is obtained. A follow up, second 12 ml blood sample is obtained either at discharge when the patient has fully recovered (at least 3 days following the event) or not later than 7 days following the episode of status epilepticus. Data is obtained from the patient's chart on medications received and the temporal relationship of medication doses, the beginning and end of the episode of status, and the time of the blood sample. Details of the episode of status, including duration of status observed, approximate duration unwitnessed (if any), clinical manifestations (convulsive or subtle), EEG findings, time of any prior episodes of status, the presence of any documented hypoxia or global ischemia, and the patient's past medical history are also obtained. The time between the end of the status epilepticus and the full recovery of normal cognitive function is documented based upon mini-mental status scores performed every 8 hours by the examining physicians. Outcome at hospital discharge will be recorded.

Adult males (n=10) and females (n=10) (all races>18 years old, <75 years old) with single generalized tonic clonic seizures are entered into this example. Patients are considered for this example if they have a history of generalized tonic clonic seizures and sample (sterile in EDTA) is obtained. A follow-up, second 12 ml blood sample is obtained within 18 to 28 hours after the patient has single generalized tonic clonic seizure. The duration, precise time of the seizure, and timing of any other seizures and their type is obtained from the patient's chart. Other information gathered will include current medication dosages and blood levels, recent changes in medications, and underlying etiology of seizures.

Approximately 30% of the patients who are admitted to inpatient epilepsy monitoring units to evaluate medically refractory seizures have events that are ultimately diagnosed as non-epileptic. These patients serve as non-epileptic controls (n=10) because they have received antiepileptic drugs prior to hospitalization and will have had those drugs tapered or discontinued during the hospitalization like the epileptic subjects. These patient have 12 ml blood samples (sterile in EDTA) obtained within 18-24 hours of admission, and have a second blood sample obtained 18-24 hours after the witnessed event that is documented by EEG criteria to have been a “non-epileptic” generalized “pseudo-seizure”.

Adult males and females (all races>18 years old, <75 years old) with syncope are entered into this example. Patients who are being evaluated for syncopal episodes by tilt table studies are considered. Each patient has a single venous 12 ml blood sample obtained. A follow-up, second 12 ml blood sample is obtained within 18-24 hours after the patient has a syncopal episode on the tilt table or as an outpatient. The duration, precise time of the syncope, and timing of any other syncopal episodes and their type and duration are obtained. Other information gathered includes current medication dosages and blood levels, recent changes in medications, and the etiology of syncope if known. Any evidence for recent severe global ischemic or anoxic events is evaluated.

RT-PCR is performed on the blood samples of all patients with status epilepticus (within 24 h of the event and then 3-7 days later); all patients with single tonic-clonic seizures (before and after the seizures); all patients with syncope (before and after the syncope); and all patients with pseudo-seizures (samples drawn before and after the event). The genes which are examined include but are not limited to: histamine H2-receptor, the c-jun leucine zipper interactive protein, Glut3, the vesicular monoamine transporter, the TNF intracellular domain interacting protein, and the vascular tyrosine phosphatase.

A pattern of expression is captured on an Affymetrix chip. Using an expression method the pattern of expression is defined for single tonic-clonic seizures (before and after the seizures); syncope (before and after the syncope); and pseudo-seizures (samples drawn before and after the event). These patterns are recorded to develop an injury database for each seizure injury. These injury databases are then used to assess the seizure in an individual.

### Example 8

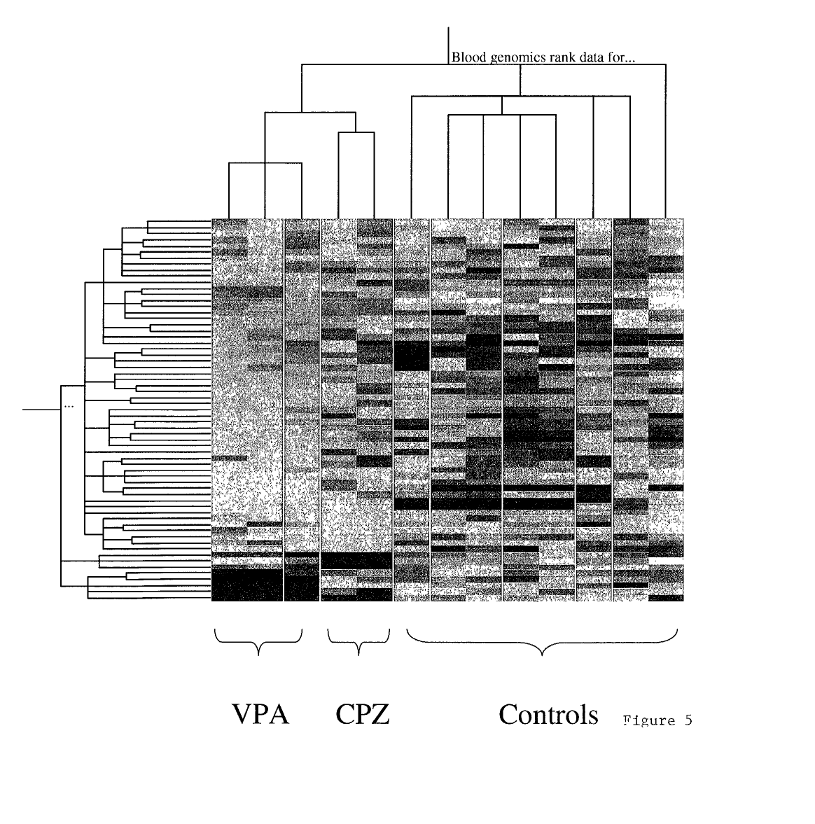

This example demonstrates that the pattern of gene expression for each drug is different from each other and different from controls. Blood is obtained from epileptic individuals, epileptic individuals being treated with anticonvulsant valporate and epileptic individuals being treated with anticonvulsant carbamazepine. A pattern of expression is captured and analyzed for each injury state as described in Example 4. As shown in FIG. 5, there are some genes upregulated for both anticonvulsants and some genes that are downregulated for both anticonvulsants, but the pattern of expression for each drug is different from each other and different from the controls, the epileptic individuals taking no anticonvulsant.

The data below demonstrates the pattern of expression for valporate and carbamazepine. Table 8a and 8b give lists of genes upregulated or downregulated for valporate, while Tables 8c and 8d give lists of genes upregulated or downregulated for carbamazepine. This data demonstrates how the pattern of expression in the blood of individuals is unique and can be used to asses toxicity or efficacy for a drug or treatment in an individual.

TABLE 8a
Upregulated genes for Valporate

| Genbank | Description |
|---|---|
| M99487 | M99487/FEATURE = /DEFINITION = |
| | HUMPSM Human prostate-specific membrane |
| | antigen (PSM) mRNA, complete cds |
| AB023162 | Homo sapiens mRNA for KIAA0945 protein, |
| | complete cds |
| X14329 | Human mRNA for carboxypeptidase N small |
| | subunit (EC 3.4.17.3) |
| X80907 | X80907/FEATURE = /DEFINITION = |
| | HSPHOSINK H. sapiens mRNA for p85 beta |
| | subunit of phosphatidyl-inositol-3-kinase |
| AJ001873 | Homo Sapiens mRNA, partial cDNA sequence |
| | from cDNA selection, DCR1-16.0 |
| M26683 | M26683/FEATURE = /DEFINITION = |
| | HUMIFNIND Human interferon gamma |
| | treatment inducible mRNA |
| L20861 | Homo sapiens proto-oncogene (Wnt-5a) mRNA, |
| | complete cds |
| AF015124 | Homo sapiens IgG heavy chain variable region |
| | (Vh26) mRNA, partial cds |
| AI373743 | qz54c04.x1 Homo sapiens cDNA, 3 end |
| AF041339 | Homo sapiens homeodomain protein (PITX3) |
| | mRNA, complete cds |
| AF031469 | Homo sapiens MHC class I related protein 1 |
| | isoform D (MR1D) mRNA, complete cds |
| AF005361 | AF005361/FEATURE = /DEFINITION = |
| | HUMIMPA6 Homo sapiens importin alpha 6 |
| | mRNA, complete cds |
| AB011089 | Homo sapiens mRNA for KIAA0517 protein, |
| | partial cds |
| D83407 | ZAKI-4 mRNA in human skin fibroblast, |
| | complete cds |
| D83784 | Human mRNA for KIAA0198 gene, partial cds |
| U93917 | Human glycine receptor alpha 3 subunit mRNA, |
| | complete cds |
| L05147 | Human dual specificity phosphatase tyrosine |
| M64554 | Human factor XIII b subunit gene, complete cds |
| J03930 | Human intestinal alkaline phosphatase (ALPI) |
| | gene, complete cds |
| AL049242 | Homo sapiens mRNA; cDNA DKFZp564B083 |
| | (from clone DKFZp564B083) |
| AL022165 | dJ71L16.5 (KIAA0267 LIKE putative Na(+) |
| W27967 | 40b10 Homo sapiens cDNA |
| AL109716 | Homo sapiens mRNA full length insert cDNA |
| | clone EUROIMAGE 208948 |
| AB007913 | Homo sapiens mRNA for KIAA0444 protein, |
| | partial cds |
| D32202 | Human mRNA for alpha 1C adrenergic receptor |
| | isoform 2, complete cds |
| AF034956 | Homo sapiens RAD51D mRNA, complete cds |
| AF093420 | Homo sapiens Hsp70 binding protein HspBP1 |
| | mRNA, complete cds |
| W30959 | zc65h10.r1 Homo sapiens cDNA, 5 end |
| D86640 | Homo sapiens mRNA for stac, complete cds |
| AB020640 | Homo sapiens mRNA for KIAA0833 protein, |
| | partial cds |
| U58090 | Human Hs-cul-4A mRNA, partial cds |
| U13022 | Human negative regulator of programmed cell |
| | death ICH-1S (Ich-1) mRNA, complete cds |
| S82075 | S82075/FEATURE = /DEFINITION = S82075 |
| | PA4 = candidate oncogene {3 region} |
| | [human, HEN-16, HEN-16T transformed endo- |
| | cervical cell lines, mRNA Partial, 315 nt] |
| AB025186 | Homo sapiens mRNA for EB3 protein, |
| | complete cds |
| U02082 | Human guanine nucleotide regulatory protein |
| | (tim1) mRNA, complete cds |
| L15309 | Human zinc finger protein (ZNF141) mRNA, |
| | complete cds |
| X83127 | H. sapiens mRNA for voltage gated potassium |
| | channels, beta subunit |
| AC004770 | Homo sapiens chromosome 11, BAC CIT-HSP- |
| | 311e8 (BC269730) containing the hFEN1 gene |
| U83598 | U83598/FEATURE = /DEFINITION = |
| | HSU83598 Human death domain receptor 3 |
| | soluble form (DDR3) mRNA, partial cds |
| U81787 | U81787/FEATURE = /DEFINITION = |
| | HSU81787 Human Wnt10B mRNA, complete cds |
| W26334 | 26b1 Homo sapiens cDNA |
| AF009242 | Homo sapiens proline-rich Gla protein 1 (PRGP1) |
| | mRNA, complete cds |
| AI307607 | tb15h10.x1 Homo sapiens cDNA, 3 end |
| M59499 | Human lipoprotein-associated coagulation |
| | inhibitor (LACI) gene |
| X96584 | H. sapiens mRNA for NOV protein |
| U71087 | U71087/FEATURE = /DEFINITION = |
| | HSU71087 Human MAP kinase kinase |
| | MEK5b mRNA, complete cds |
| M35198 | M35198/FEATURE = /DEFINITION = |
| | HUMINTB6A Human integrin B-6 mRNA, |
| | complete cds |
| AF025304 | Homo sapiens protein-tyrosine kinase EPHB2v |
| | (EPHB2) mRNA, complete cds |
| AC005053 | Homo sapiens BAC clone RG041D11 from 7q21 |
| D17291 | Human gene for regenerating protein I beta, |
| | complete cds |
| U28687 | Human zinc finger containing protein ZNF157 |
| | (ZNF157) mRNA, complete cds |
| D26535 | Human gene for dihydrolipoamide |
| | succinyltransferase, complete cds (exon 1-15) |
| L12760 | Human phosphoenolpyruvate carboxykinase |
| | (PCK1) gene, complete cds with repeats |
| U62325 | Human FE65-like protein (hFE65L) mRNA, |
| | partial cds |
| AB006624 | Homo sapiens mRNA for KIAA0286 gene, |
| | partial cds |
| D14539 | Human mRNA for LTG19 |
| U52112 | neural cell adhesion molecule L1 |
| AL080140 | Homo sapiens mRNA; cDNA DKFZp434L243 |
| | (from clone DKFZp434L243) |
| U19977 | Human preprocarboxypeptidase A2 (proCPA2) |
| | mRNA, complete cds |
| AA418437 | zv92d11.r1 Homo sapiens cDNA, 5 end |
| U17579 | Human growth hormone-releasing hormone |
| | receptor gene, alternatively spliced |
| | forms a, b, and c, partial cds |
| X82634 | Homo sapiens mRNA for hair keratin acidic 3-II |
| AL080175 | Homo sapiens mRNA; cDNA DKFZp434K091 |
| | (from clone DKFZp434K091) |
| M20919 | Human DNA with a hepatitis B virus surface |
| | antigen (HBsAg) gene (complete cds) insertion |
| AA733050 | zg79b05.s1 Homo sapiens cDNA, 3 end |
| Z78388 | HSZ78388 Homo sapiens cDNA |
| AI819249 | wj42f05.x1 Homo sapiens cDNA, 3 end |
| AB011147 | Homo sapiens mRNA for KIAA0575 protein, |
| | complete cds |
| AF097935 | Homo sapiens desmoglein 1 (DSG1) mRNA, |
| | complete cds |
| AB004848 | Homo sapiens mRNA expressed in placenta, |
| | clone IMAGE-70506 |
| P97Antigen, | P97 Antigen, Melanoma-Specific |
| Melanoma-Specific | |
| D87463 | Human mRNA for KIAA0273 gene, complete cds |
| AF052150 | Homo sapiens clone 24533 mRNA sequence |
| M64929 | Human protein phosphatase 2A alpha subunit |
| | mRNA, complete cds |
| AF045941 | Homo sapiens sciellin (SCEL) mRNA, |
| | complete cds |
| AB028996 | Homo sapiens mRNA for KIAA1073 protein, |
| | complete cds |
| M68520 | M68520/FEATURE = /DEFINITION = |
| | HUMCDC2A Human cdc2-related protein kinase |
| | mRNA, complete cds |
| Helix-Loop- | Helix-Loop-Helix Protein Delta Max, Alt. |
| HelixProteinDeltaMax, | Splice 1 |
| Alt.Splice1 | |
| AI985019 | wu44a10.x1 Homo sapiens cDNA, 3 end |
| AF035314 | Homo sapiens clone 23651 mRNA sequence |
| AB023157 | Homo sapiens mRNA for KIAA0940 protein, |
| | complete cds |
| X51630 | X51630/FEATURE = mRNA/DEFINITION = |
| | HSWT1 Human Wilms tumor WT1 mRNA for |
| | zinc finger protein, Krueppel-like |
| AB018349 | Homo sapiens mRNA for KIAA0806 protein, |
| | complete cds |
| U02632 | Human calcium-activated potassium channel |
| | mRNA, partial cds |
| J05096 | Human Na, K-ATPase subunit alpha 2 (ATP1A2) |
| | gene, complete cds |
| D79995 | Human mRNA for KIAA0173 gene, complete cds |
| U66582 | Human gammaC-crystallin (CRYGC) mRNA, |
| | complete cds |
| U43527 | U43527/FEATURE = /DEFINITION = |
| | HSU43527 Human malignant melanoma |
| | metastasis-suppressor (KiSS-1) gene, mRNA, |
| | complete cds |
| M60299 | M60299/FEATURE = cds/DEFINITION = |
| | HUMCOLII Human alpha-1 collagen type II |
| | gene, exons 1, 2 and 3 |
| L08488 | L08488/FEATURE = /DEFINITION = |
| | HUMINOS Human inositol polyphosphate |
| | 1-phosphatase mRNA, complete cds |
| AL022718 | dJ1052M9.3 (mouse DOC4 LIKE protein) |
| W03846 | za60a02.r1 Homo sapiens cDNA, 5 end |
| AF012130 | Homo sapiens brachyury variant A (TBX1) |
| | mRNA, complete cds |
| AF075292 | Homo sapiens fibroblast growth factor 18 |
| | (FGF18) mRNA, complete cds |
| D43772 | D43772/FEATURE = /DEFINITION = |
| | HUMGRB7 Human squamous cell carcinama |
| | of esophagus mRNA for GRB-7 SH2 domain |
| | protein, complete cds |
| X13967 | X13967/FEATURE = cds/DEFINITION = |
| | HSLIF Human mRNA for leukaemia |
| | inhibitory factor (LIF/HILDA) |
| AF041210 | Homo sapiens midline 1 fetal kidney isoform 3 |
| | (MID1) mRNA, partial cds |
| | X07876/FEATURE = cds/DEFINITION = |
| | HSIRP Human mRNA for irp protein (int-1 |
| | related protein)/NOTE = replacement of probe |
| | set 439_at |
| U76366 | Human Treacher Collins syndrome (TCOF1) |
| | mRNA, complete cds |
| RetinoicAcidReceptor, | Retinoic Acid Receptor, Gamma 2 |
| Gamma 2 | |
| W28161 | 42h10 Homo sapiens cDNA |
| X99688 | H. sapiens mRNA from TYL gene |
| W26805 | 13a12 Homo sapiens cDNA |
| W26019 | 18b9 Homo sapiens cDNA |
| AI828210 | wk81e09.x1 Homo sapiens cDNA, 3 end |
| U79725 | Human A33 antigen precursor mRNA, |
| | complete cds |
| AL109722 | Homo sapiens mRNA full length insert cDNA |
| | clone EUROIMAGE 31619 |
| AB014544 | Homo sapiens mRNA for KIAA0644 protein, |
| | complete cds |
| W27763 | 37c8 Homo sapiens cDNA |
| D12763 | Homo sapiens mRNA for ST2 protein |
| X84003 | H. sapiens TAFII18 mRNA for transcription |
| | factor TFIID |
| S66666 | p53 = tumor suppressor {alternatively spliced, |
| | exon 9-10} [human, Molt-4, T-lymphoblastic |
| | leukemia cell line, mRNA PartialMutant, 160 nt] |
| AF077954 | Homo sapiens protein inhibitor of activated STAT |
| | protein PIASx-beta mRNA, complete cds |
| R37702 | yf50d02.s1 Homo sapiens cDNA, 3 end |
| AA418080 | zv97h07.s1 Homo sapiens cDNA, 3 end |
| AB028994 | Homo sapiens mRNA for KIAA1071 protein, |
| | partial cds |
| Z26308 | H. sapiens isoform 1 gene for L-type calcium |
| | channel, neuronal subform (partial) |
| AB003592 | Homo sapiens mRNA for neural adhesion |
| | molecule NB-3, complete cds |
| M77348 | Human Pmel 17 mRNA, complete cds |
| U15306 | Human cysteine-rich sequence-specific DNA- |
| | binding protein NFX1 mRNA, complete cds |
| AI880840 | at11d06.x1 Homo sapiens cDNA, 3 end |
| AB006651 | Homo sapiens EXLM1 mRNA, complete cds |
| Z19585 | Z19585/FEATURE = cds/DEFINITION = |
| | HSTHROMB4 H. sapiens mRNA for |
| | thrombospondin-4 |
| U50535 | U50535/FEATURE = /DEFINITION = |
| | HSU50535 Human BRCA2 region, mRNA |
| | sequence CG006 |
| M85164 | Homo sapiens SRF accessory protein 1B (SAP-1) |
| | mRNA, complete cds |
| V01510 | H. sapiens gene coding for ACTH and beta-LPH |
| | precursors. Gene codes for the common precursor |
| | of the pituitary hormones corticotropin (ACTH) |
| | and beta-lipotropin (beta-LPH) |
| U66048 | Human clone 161455-2-3 B cell expressed mRNA |
| | from chromosome X |
| AB024729 | Homo sapiens hGnT-IV-H mRNA for alpha- |
| | 1,3-D-mannoside beta-1,4-N-acetylglucosaminyl- |
| | transferase IV-homologue, complete cds |
| AJ0001634 | Homo sapiens mRNA for CC-chemokine MCP-4 |
| AF052186 | Homo sapiens clone 24431 mRNA sequence |
| AF084535 | Homo sapiens laforin (EPM2A) mRNA, |
| | partial cds |
| U20982 | Human insulin-like growth factor binding |
| | protein-4 (IGFBP4) gene, promoter and |
| | complete cds |
| L32164 | Homo sapiens zinc finger protein mRNA, 3 end |
| X16866 | X16866/FEATURE = /DEFINITION = |
| | HSP450II Human mRNA for cytochrome P- |
| | 450110 (clone pMP33) |
| AJ011733 | Homo sapiens mRNA for synaptogyrin 4 protein |
| X77533 | H. sapiens mRNA for activin type II receptor |
| U16861 | Human inward rectifying potassium channel |
| | mRNA, complete cds |
| X99141 | H. sapiens mRNA for hair keratin, hHb3 |
| D86962 | Human mRNA for KIAA0207 gene, complete cds |
| AI936759 | wp69b12.x1 Homo sapiens cDNA, 3 end |
| X99947 | Homo sapiens mRNA dynein-related protein |
| AL050287 | Homo sapiens mRNA; cDNA DKFZp586C021 |
| | (from clone DKFZp586C021) |
| AF070628 | Homo sapiens clone 24803 mRNA sequence |
| AJ011123 | Homo sapiens mRNA for phosphatidylinositol |
| | 4-kinase (NPIK-C) |

TABLE 8b
Downregulated genes for Valporate

| Genbank | Description |
|---|---|
| AB014514 | Homo sapiens mRNA for KIAA0614 protein, partial cds |
| AF015767 | Homo sapiens brain and reproductive organ-expressed |
| | protein (BRE) mRNA, complete cds |
| AF038564 | Homo sapiens atrophin-1 interacting protein 4 (AIP4) |
| | mRNA, partial cds |
| X62055 | X62055/FEATURE = cds/DEFINITION = HSPTP1C |
| | H. sapiens PTP1C mRNA for protein-tyrosine |
| | phosphatase C |
| AB001740 | Homo sapiens mRNA for p27, complete cds |
| X16901 | Human mRNA for RAP30 subunit of transcription initiation |
| | factor RAP30 |
| U10324 | Human nuclear factor NF90 mRNA, complete cds |
| AL022326 | dJ333H23.2.2 (Synaptogyrin 1A (SYNGR1A)) |
| AA552988 | nk83d08.s1 Homo sapiens cDNA, 3 end |
| L13616 | Human focal adhesion kinase (FAK) mRNA, complete cds |
| X59656 | X59656/FEATURE = cds/DEFINITION = HSCRKL |
| | H. sapiens crk-like gene CRKL |
| U92817 | Homo sapiens unnamed HERV-H protein mRNA, |
| | complete cds |
| X70218 | X70218/FEATURE = /DEFINITION = HSPPX |
| | Homo sapiens mRNA for protein phosphatase X |
| AF030427 | Homo sapiens lung type-I cell membrane-associated protein |
| | hT1a-1 (hT1a-1) mRNA, complete cds |
| M37238 | M37238/FEATURE = mRNA/DEFINITION = HUMPLC |
| | Human phospholipase C mRNA, complete cds |
| D11151 | D11151/FEATURE = _expandCDS/DEFINITION = |
| | HUMETAR8 Human DNA for endothelin-A receptor, exon |
| | 8 and 3 flanking region |
| AB018258 | Homo sapiens mRNA for KIAA0715 protein, partial cds |
| M69043 | M69043/FEATURE = /DEFINITION = |
| | HUMMAD3A Homo sapiens MAD-3 mRNA encoding |
| | lkB-like activity, complete cds |
| AL050395 | Homo sapiens mRNA; cDNA DKFZp586D1020 (from |
| | clone DKFZp586D1020) |
| X73608 | H. sapiens mRNA for testican |
| D26362 | Human mRNA for KIAA0043 gene, complete cds |
| X06318 | X06318/FEATURE = cds/DEFINITION = |
| | HSPKCB1A Human mRNA for protein kinase C (PKC) |
| | type beta I |
| R54564 | yg81b12.s1 Homo sapiens cDNA, 3 end |
| D80008 | Human mRNA for KIAA0186 gene, complete cds |
| D88799 | D88799/FEATURE = /DEFINITION = D88799 |
| | Homo sapiens mRNA for cadherin, partial cds |
| U02570 | U02570/FEATURE = /DEFINITION = HSU02570 |
| | Human CDC42 GTPase-activating protein mRNA, |
| | partial cds |
| U49392 | Human allograft inflammatory factor-1 AIF-1 mRNA, |
| | complete cds |
| U84894 | Human 239AB mRNA, complete cds |
| Y12851 | Homo sapiens P2X7 gene, exon 1 and joined CDS |
| D42123 | Homo sapiens mRNA for ESP1 |
| AF070585 | Homo sapiens clone 24675 mRNA sequence |

TABLE 8c
Upregulated genes for Carbamazepine

| Genbank | Description |
|---|---|
| AB000824 | Homo sapiens mRNA for trehalase, complete cds |
| AA883870 | am26f01.s1 Homo sapiens cDNA, 3 end |
| L18920 | Human MAGE-2 gene exons 1-4, complete cds |
| Z19585 | Z19585/FEATURE = cds/DEFINITION = HSTHROMB4 |
| | H. sapiens mRNA for thrombospondin-4 |
| U83410 | Human CUL-2 (cul-2) mRNA, complete cds |
| L34838 | Homo sapiens early placenta insulin-like peptide EPIL |
| | (INSL4) mRNA, complete cds |
| U16258 | U16258/FEATURE = /DEFINITION = HSU16258 Human |
| | I kappa BR mRNA, complete cds |
| X02750 | Human liver mRNA for protein C |
| U27516 | U27516/FEATURE = /DEFINITION = HSU27516 Human |
| | recombination protein RAD52 mRNA, complete cds |
| M35296 | M35296/FEATURE = /DEFINITION = HUMARGCAA |
| | Human tyrosine kinase arg gene mRNA |

TABLE 8d
Downregulated genes for Carbamazepine

| Genbank | Description |
|---|---|
| AB014514 | Homo sapiens mRNA for KIAA0614 protein, partial cds |
| AF015767 | Homo sapiens brain and reproductive organ-expressed |
| | protein (BRE) mRNA, complete cds |
| AF038564 | Homo sapiens atrophin-1 interacting protein 4 (AIP4) |
| | mRNA, partial cds |
| X62055 | X62055/FEATURE = cds/DEFINITION = HSPTP1C H. |
| | sapiens PTP1C mRNA for protein-tyrosine phosphatase 1C |
| AB001740 | Homo sapiens mRNA for p27, complete cds |
| X16901 | Human mRNA for RAP30 subunit of transcription initiation |
| | factor RAP30 |
| U10324 | Human nuclear factor NF90 mRNA, complete cds |
| AL022326 | dJ333H23.2.2 (Synaptogyrin 1A (SYNGR1A)) |
| AA552988 | nk83d08.s1 Homo sapiens cDNA, 3 end |
| L13616 | Human focal adhesion kinase (FAK) mRNA, complete cds |
| X59656 | X59656/FEATURE = cds/DEFINITION = HSCRKL |
| | H. sapiens crk-like gene CRKL |
| U92817 | Homo sapiens unnamed HERV-H protein mRNA, |
| | complete cds |
| X70218 | X70218/FEATURE = /DEFINITION = HSPPX Homo |
| | sapiens mRNA for protein phosphatase X |
| AF030427 | Homo sapiens lung type-I cell membrane-associated |
| | protein hT1a-1 (hT1a-1) mRNA, complete cds |
| M37238 | M37238/FEATURE = mRNA/DEFINITION = HUMPLC |
| | Human phospholipase C mRNA, complete cds |
| D11151 | D11151/FEATURE = _expandCDS/DEFINITION = |
| | HUMETAR8 Human DNA for endothelin-A receptor, |
| | exon 8 and 3 flanking region |
| AB018258 | Homo sapiens mRNA for KIAA0715 protein, partial cds |
| M69043 | M69043/FEATURE = /DEFINITION = HUMMAD3A |
| | Homo sapiens MAD-3 mRNA encoding lkB-like activity, |
| | complete cds |
| AL050395 | Homo sapiens mRNA; cDNA DKFZp586D1020 (from |
| | clone DKFZp586D1020) |
| X73608 | H. sapiens mRNA for testican |
| D26362 | Human mRNA for KIAA0043 gene, complete cds |
| X06318 | X06318/FEATURE = cds/DEFINITION = HSPKCB1A |
| | Human mRNA for protein kinase C (PKC) type beta I |
| R54564 | yg81b12.s1 Homo sapiens cDNA, 3 end |
| D80008 | Human mRNA for KIAA0186 gene, complete cds |
| D88799 | D88799/FEATURE = /DEFINITION = D88799 |
| | Homo sapiens mRNA for cadherin, partial cds |
| U02570 | U02570/FEATURE = /DEFINITION = HSU02570 |
| | Human CDC42 GTPase-activating protein mRNA, |
| | partial cds |
| U49392 | Human allograft inflammatory factor-1 (AIF-1) mRNA, |
| | complete cds |
| U84894 | Human 239AB mRNA, complete cds |
| Y12851 | Homo sapiens P2X7 gene, exon 1 and joined CDS |
| D42123 | Homo sapiens mRNA for ESP1 |
| AF070585 | Homo sapiens clone 24675 mRNA sequence |

### Example 9

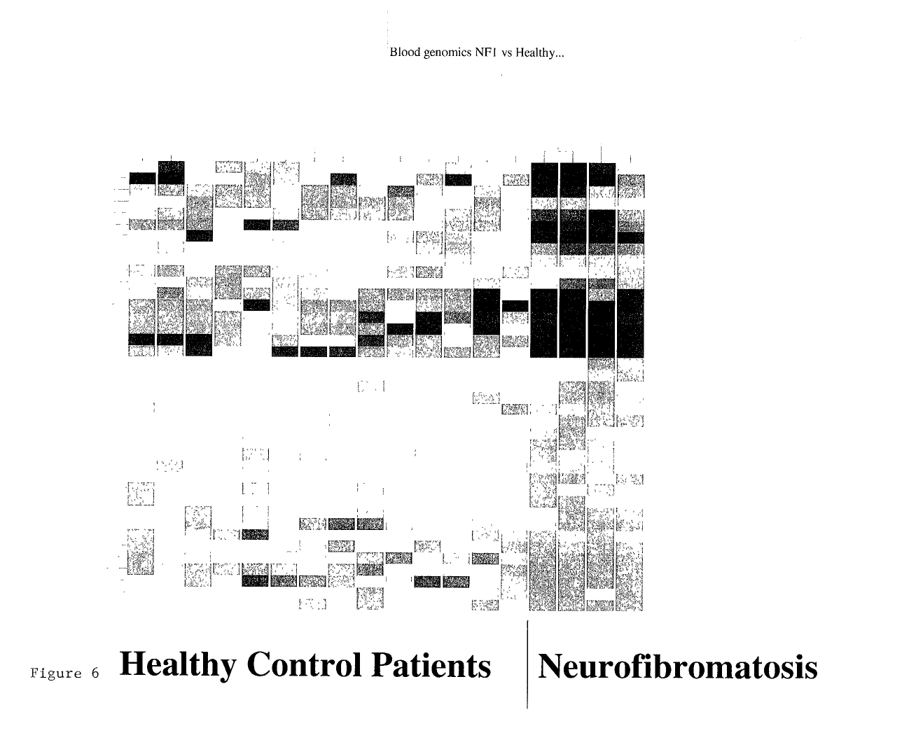

This example demonstrates that the pattern of expression for each neurofibromatosis individual as compared to individuals without neurofibromatosis. Blood is obtained from neurofibromatosis individuals and individuals without neurofibromatosis. The patterns of expressions are captured and analyzed as described in Example 4. As shown in FIG. 6, there is a defined pattern of expression for neurofibromatosis individuals that is different from individuals without neurofibromatosis.

The data below demonstrates the pattern of expression for neurofibromatosis. Table 9a and 9b give lists of genes upregulated or downregulated for neurofibromatosis. This data demonstrates how the pattern of expression in the blood of individuals is unique and can be used to assess proliferative injury including neurofibromatosis, in an individual.

TABLE 9a
Upregulated genes

| Genbank | Description |
|---|---|
| M91368 | Human Na+ |
| Z83838 | Human DNA sequence from PAC 127B20 on chromosome |
| | 22q11.2-qter, contains gene for GTPase-activating protein |
| | similar to rhoGAP protein. ribosomal protein L6 |
| | pseudogene, ESTs and CA repeat |
| V01512 | V01512/FEATURE = mRNA#1/DEFINITION = HSCFOS |
| | Human cellular oncogene c-fos (complete sequence) |
| V01512 | V01512/FEATURE = mRNA#2/DEFINITION = HSCFOS |
| | Human cellular oncogene c-fos (complete sequence) |
| AI275093 | q165c10.x1 Homo sapiens cDNA, 3 end |
| AF034633 | Homo sapiens orphan G protein-coupled receptor (GPR39) |
| | mRNA, complete cds |
| U59863 | Human TRAF-interacting protein I-TRAF mRNA, |
| | complete cds |
| AF011468 | Homo sapiens serine |
| AB014515 | Homo sapiens mRNA for KIAA0615 protein, complete cds |
| M89470 | Human paired-box protein (PAX2) mRNA, complete cds |
| AB011141 | Homo sapiens mRNA for KIAA0569 protein, complete cds |
| U70987 | U70987/FEATURE = /DEFINITION = HSU70987 |
| | Human GAP binding protein p62dok (DOK) mRNA, |
| | complete cds |
| M22995 | Human ras-related protein (Krev-1) mRNA, complete cds |
| U55184 | Human G protein Golf alpha gene |
| U81523 | Human endometrial bleeding associated factor mRNA, |
| | complete cds |
| S81439 | S81439/FEATURE = /DEFINITION = S81439 EGR |
| | alpha = early growth response gene alpha [human, prostate, |
| | mRNA, 3228 nt] |
| D79989 | Human mRNA for KIAA0167 gene, complete cds |
| Y11251 | H. sapiens mRNA for novel member of serine-arginine |
| | domain protein, SRrp129 |
| AB028956 | Homo sapiens mRNA for KIAA1033 protein, |
| | partial cds |
| Z36531 | H. sapiens mRNA for fibrinogen-like protein |
| | (pT49 protein) |
| AF078544 | Homo sapiens brain mitochondrial carrier protein-1 |
| | (BMCP1) mRNA, nuclear gene encoding mitochondrial |
| | protein, complete cds |
| M76446 | Human alpha-A1-adrenergic receptor mRNA, complete cds |
| U04636 | U04636/FEATURE = mRNA/DEFINITION = HSU04636 |
| | Human cyclooxygenase-2 (hCox-2) gene, complete cds |
| X61118 | Human TTG-2 mRNA for a cysteine rich protein with LIM |
| | motif |
| K00650 | K00650/FEATURE = cds/DEFINITION = HUMFOS |
| | Human fos proto-oncogene (c-fos), complete cds |
| AB007945 | Homo sapiens mRNA for KIAA0476 protein, complete cds |
| D38524 | D38524/FEATURE = /DEFINITION = HUM5N |
| | Human mRNA for 5-nucleotidase |
| AB018276 | Homo sapiens mRNA for KIAA0733 protein, partial cds |
| AF088219 | Homo sapiens CC chemokine gene cluster, complete |
| | sequence |
| AL008583 | dJ327J16.1 (human ortholog of mouse outer arm Dynein |
| | light chain 4) |
| M24283 | Human major group rhinovirus receptor (HRV) mRNA, |
| | complete cds |
| AB013382 | Homo sapiens mRNA for DUSP6, complete cds |
| U67322 | Human HBV associated factor (XAP4) mRNA, |
| | complete cds |
| U06698 | Human neuronal kinesin heavy chain mRNA, complete cds |
| X03168 | Human mRNA for S-protein |
| X78711 | H. sapiens mRNA for glycerol kinase testis specific 1 |
| AF025530 | Homo sapiens leucocyte immunoglobulin-like receptor-6a |
| | (LIR-6) mRNA, complete cds |
| AF051426 | Homo sapiens slow delayed rectifier channel subunit |
| | mRNA, complete cds |
| U95735 | Human SNARE protein Ykt6 (YKT6) mRNA, complete cds |
| U43519 | Human dystrophin-related protein 2 (DRP2) mRNA, |
| | complete cds |
| D80005 | Human mRNA for KIAA0183 gene, partial cds |
| AL050145 | Homo sapiens mRNA; cDNA DKFZp586C2020 (from |
| | clone DKFZp586C2020) |
| X51345 | Human jun-B mRNA for JUN-B protein |
| AW005997 | wz91c01.x1 Homo sapiens cDNA, 3 end |
| L23805 | L23805/FEATURE = /DEFINITION = HUMCATENIN |
| | Human alpha1 (E)-catenin mRNA, complete cds |
| X54637 | X54637/FEATURE = cds/DEFINITION = HSTYK2 |
| | Human tyk2 mRNA for non-receptor protein tyrosine |
| | kinase |
| Y11731 | H. sapiens mRNA for DNA glycosylase |
| M76125 | M76125/FEATURE = /DEFINITION = HUMTYRKINR |
| | Human tyrosine kinase receptor (axl) mRNA, complete cds |
| L28957 | Homo sapiens CTP-phosphocholine cytidyltransferase |
| | mRNA, complete cds |
| U64520 | Human synaptobrevin-3 mRNA, complete cds |
| AL021808 | Human DNA sequence from clone 24o18 on chromosome |
| | 6p21.31-22.2 Contains zinc finger protein pseudogene, |
| | VNO-type olfactory receptor pseudogene, nuclear envelope |
| | pore membrane protein, EST, STS, GSS |
| X68880 | H. sapiens EMX2 mRNA |
| L29254 | Human (clone P1-5) L-iditol-2 dehydrogenase gene |
| AF051323 | Homo sapiens Src-associated adaptor protein (SAPS) |
| | mRNA, complete cds |
| M29039 | M29039/FEATURE = cds/DEFINITION = HUMJUNCAA |
| | Human transactivator jun-B) gene, complete cds |
| AI375610 | ta08f06.x1 Homo sapiens cDNA, 3 end |
| AF060219 | Homo sapiens RCC1-like G exchanging factor RLG |
| | mRNA, complete cds |
| S74017 | S74017/FEATURE = /DEFINITION = S74017 Nrf2 = |
| | NF-E2-like basic leucine zipper transcriptional activator |
| | [human, hemin-induced K562 cells, mRNA, 2304 nt] |
| U01923 | Human BTK region clone ftp-3 mRNA |
| X71874 | X71874/FEATURE = cds#2/DEFINITION = |
| | HSPROSCHY H. sapiens genes for proteasome-like subunit |
| | (MECL-1), chymotrypsin-like protease (CTRL-1) and |
| | protein serine kinase (P5K-H1) last exon |
| U03100 | Human alpha2(E)-catenin mRNA, complete cds |

TABLE 9b
Down regulated genes

| Genbank | Description |
|---|---|
| AF009624 | Homo sapiens K1F3-related motor protein (KIF3X) mRNA, |
| | partial cds |
| X97671 | X97671/FEATURE = cds/DEFINITION = HSERYTHR |
| | H. sapiens mRNA for erythropoietin receptor |
| X91348 | H. sapiens predicted non coding cDNA (DGCR5) |
| X68679 | H. sapiens mRNA for DOWN 16 |
| Z37986 | H. sapiens mRNA for phenylalkylamine binding protein |
| AF007871 | Homo sapiens torsinA (DYT1) mRNA, complete cds |
| W27191 | 23e6 Homo sapiens cDNA |
| Z98265 | Homo sapiens mRNA for plakophilin 3 |
| J04132 | Human T cell receptor zeta-chain mRNA, complete cds |
| AA885106 | am31h01.s1 Homo sapiens cDNA, 3 end |
| AL120500 | DKFZp761M078_s1 Homo sapiens cDNA, 3 end |
| D85245 | Homo sapiens mRNA for TR3beta, complete cds |
| U79115 | U79115/FEATURE = /DEFINITION = HSU79115 |
| | Human death adaptor molecule RAIDD (RAIDD) mRNA, |
| | complete cds |
| AF048713 | Homo sapiens Kv4.3 potassium channel long splice |
| | variant (Kv4.3) mRNA, complete cds |
| M64716 | Human ribosomal protein S25 mRNA, complete cds |
| U01038 | Human pLK mRNA, complete cds |
| AF047715 | Homo sapiens A-kinase anchoring protein (AKAP18) |
| | mRNA, complete cds |
| U43195 | Human Rho-associated, coiled-coil containing protein |
| | kinase p160ROCK mRNA, complete cds |
| U18550 | Human GPR3 G protein-coupled receptor gene, |
| | complete cds |
| W28616 | 49b9 Homo sapiens cDNA |
| X72631 | H. sapiens mRNA encoding Rev-ErbAalpha |
| AF059198 | Homo sapiens protein kinase |
| J04423 | J04423 E coli bioB gene biotin synthetase (−5, −M, −3 |
| | represent transcript regions 5 prime, Middle, and 3 prime |
| | respectively) |
| U50535 | U50535/FEATURE = /DEFINITION = HSU50535 |
| | Human BRCA2 region, mRNA sequence CG006 |
| U15782 | Human cleavage stimulation factor 77 kDa subunit mRNA, |
| | complete cds |
| X90872 | H. sapiens mRNA for gp25L2 protein |
| U09577 | Homo sapiens lysosomal hyaluronidase (LUCA2 |
| AL049415 | Homo sapiens mRNA; cDNA DKFZp586N2119 (from |
| | clone DKFZp586N2119) |
| H16917 | ym39e02.r1 Homo sapiens cDNA, 5 end |
| AB007510 | Homo sapiens mRNA for PRP8 protein, complete cds |
| X03453 | X03453/description = Bacteriophage P1 ORF2, putatitve |
| | cre protein |
| AI968364 | wu02c08.x1 Homo sapiens cDNA, 3 end |
| AF088219 | Homo sapiens CC chemokine gene cluster, complete |
| | sequence |
| J04423 | J04423 E coli bioB gene biotin synthetase (−5, −M, −3 |
| | represent transcript regions 5 prime, Middle, and 3 prime |
| | respectively) |
| D29805 | Human mRNA for beta-1,4-galactosyltransferase, |
| | complete cds |
| X74328 | H. sapiens mRNA for CB2 (peripheral) cannabinoid |
| | receptor |
| AF026291 | Homo sapiens chaperonin containing t-complex polypeptide |
| | 1, delta subunit (Cctd) mRNA, complete cds |
| Y00097 | Human mRNA for protein p68 |
| AI332820 | qp96e06.x1 Homo sapiens cDNA, 3 end |
| X73114 | H. sapiens mRNA for slow MyBP-C |
| U29615 | Human chitotriosidase precursor mRNA, complete cds |
| J04423 | J04423 E coli bioB gene biotin synthetase (−5, −M, −3 |
| | represent transcript regions 5 prime, Middle, and 3 prime |
| | respectively) |
| Y15801 | Homo sapiens mRNA for PRKY protein |
| AB020706 | Homo sapiens mRNA for KIAA0899 protein, partial cds |
| S69115 | granulocyte colony-stimulating factor induced gene |
| | [human, CML patient, bone marrow mononuclear cells, |
| | mRNA, 833 nt] |
| U68487 | Human 5-hydroxytryptamine7 receptor isoform b mRNA, |
| | complete cds |
| AL109696 | Homo sapiens mRNA full length insert cDNA clone |
| | EUROIMAGE 21920 |
| AF000571 | Homo sapiens kidney and cardiac voltage dependent K+ |
| | channel (KvLQT1) mRNA, complete cds |
| M19309 | Human slow skeletal muscle troponin T mRNA, |
| | clone H22h |
| AJ237672 | Homo sapiens mRNA for methylenetetrahydrofolate |
| | reductase |
| U80735 | Homo sapiens CAGF28 mRNA, partial cds |
| X04688 | X04688/FEATURE = cds/DEFINITION = HSIL5R |
| | Human mRNA for T-cell replacing factor (interleukin-5) |
| D86956 | Human mRNA for KIAA0201 gene, complete cds |
| X58199 | Human mRNA for beta adducin |
| U86214 | U86214/FEATURE = /DEFINITION = HSU86214 |
| | Human Fas-associated death domain protein interleukin- |
| | 1b-converting enzyme 2 mRNA, complete cds |
| AI553878 | tn30a05.x1 Homo sapiens cDNA, 3 end |
| X90763 | Homo sapiens mRNA for type I keratin |
| AB014535 | Homo sapiens mRNA for KIAA0635 protein, complete cds |
| AJ012611 | Homo sapiens mRNA for SIX3 protein |
| M31651 | Homo sapiens sex hormone-binding globulin (SHBG) gene, |
| | complete cds |
| AB028967 | Homo sapiens mRNA for KIAA1044 protein, complete cds |
| X13293 | X13293/FEATURE = cds/DEFINITION = HSBMYB |
| | Human mRNA for B-myb gene |
| J03407 | Human rfp transforming protein mRNA, complete cds |
| D17427 | Human mRNA for desmocollin type 4 |
| AL049280 | Homo sapiens mRNA; cDNA DKFZp564K143 (from clone |
| | DKFZp564K143) |
| U73394 | Human NK-receptor (KIR-103AST) mRNA, complete cds |
| U67369 | Human growth factor independence-1 (Gfi-1) mRNA, |
| | complete cds |
| X91148 | H. sapiens mRNA for microsomal triglyceride transfer |
| | protein |
| X97229 | H. sapiens mRNA for NK receptor, clone library 15.212 |
| AB014581 | Homo sapiens mRNA for KIAA0681 protein, partial cds |
| M73628 | Homo sapiens kappa-casein mRNA, complete cds |
| AF052145 | Homo sapiens clone 24400 mRNA sequence |
| AF090097 | Homo sapiens clone IMAGE 25997 |
| AB023177 | Homo sapiens mRNA for KIAA0960 protein, partial cds |
| X53281 | H. sapiens BTF3b mRNA |
| L78440 | L78440/FEATURE = mRNA/DEFINITION = |
| | HUMSTAT4R Homo sapiens STAT4 mRNA, complete cds |
| U11276 | Human hNKR-P1a protein (NKR-P1A) mRNA, |
| | complete cds |
| AB018258 | Homo sapiens mRNA for KIAA0715 protein, |
| | partial cds |
| M98539 | M98539/FEATURE = exon/DEFINITION = HUMPDS03 |
| | Human prostaglandin D2 synthase gene, exon 7 |
| AL022721 | dJ109F14.2 (60S Ribosomal Protein RPL10A) |
| Rad2 | Rad2 |
| AL050152 | Homo sapiens mRNA; cDNA DKFZp586K1220 (from |
| | clone DKFZp586K1220) |
| U47025 | Human fetal brain glycogen phosphorylase B mRNA, |
| | complete cds |
| AA464312 | zx78c11.r1 Homo sapiens cDNA, 5 end |
| X55954 | Human mRNA for HL23 ribosomal protein homologue |
| X51688 | X51688/FEATURE = mRNA/DEFINITION = |
| | HSCYCLINA Human mRNA for cyclin A |
| U09196 | Human 1.1 kb mRNA upregulated in retinoic acid treated |
| | HL-60 neutrophilic cells |
| U08438 | Human beta-adrenergic receptor kinase (ADRBK1) gene |
| X16867 | Human mRNA for cytochrome P-45011D (clone pMP34) |
| U26209 | Human renal sodium |
| X95808 | H. sapiens mRNA for protein encoded by a candidate gene, |
| | DXS6673E, for mental retardation |
| AB007895 | Homo sapiens KIAA0435 mRNA, complete cds |
| M21624 | M21624/FEATURE = mRNA/DEFINITION = |
| | HUMTCRGC Human T-cell receptor delta chain mRNA |
| | (VJC-region), complete cds |
| AI207842 | ao89h09.x1 Homo sapiens cDNA, 3 end |
| U24266 | Human pyrroline-5-carboxylate dehydrogenase (P5CDh) |
| | mRNA, long form, complete cds |

### Example 10

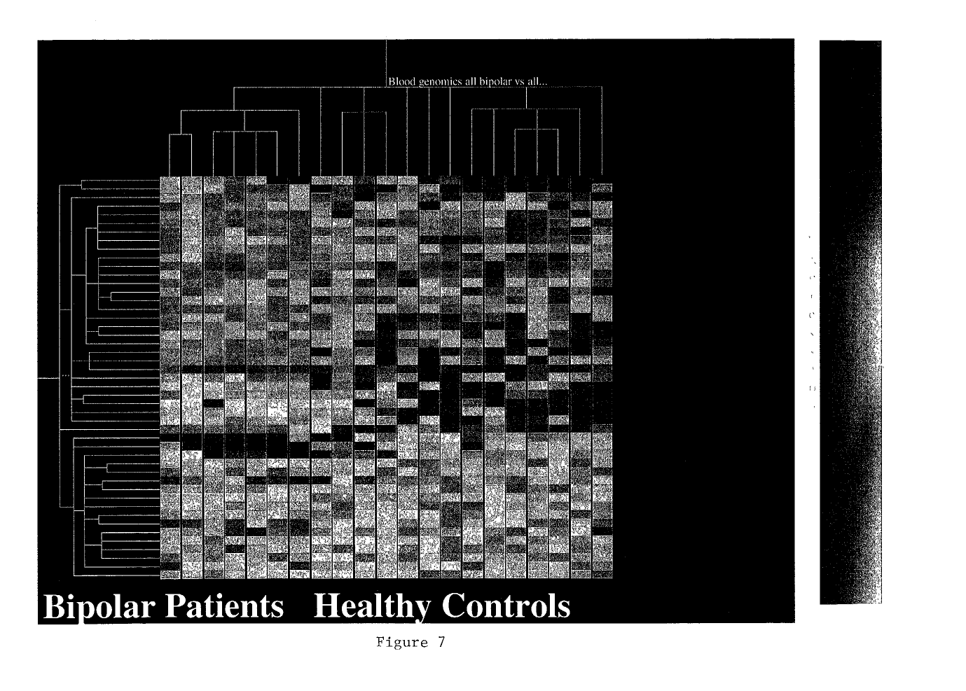

This example demonstrates that the pattern of expression for each bipolar, manic-depressive, individuals as compared to individuals without bipolar. Blood is obtained from bipolar individuals and individuals without bipolar. The patterns of expressions are captured and analyzed as described in Example 4. As shown in FIG. 7, a defined pattern of expression for bipolar individuals is determined that is different from individuals without bipolar.

The data below demonstrates the pattern of expression for bipolar. Table 10a and 10b give lists of genes upregulated or downregulated for bipolar. This data demonstrates how the pattern of expression in the blood of individuals is unique and can be used to assess psychosis, including bipolar, in an individual.

TABLE 10a
Upregulated genes

| Genbank | Description |
|---|---|
| U81787 | U81787/FEATURE = /DEFINITION = HSU81787 |
| | Human Wnt10B mRNA, complete cds |
| AF049498 | Homo sapiens sodium channel beta 2 subunit (SCN2B) |
| | mRNA, complete cds |
| M21985 | M21985/FEATURE = /DEFINITION = HUMSRTR2A |
| | Human steroid receptor TR2 mRNA, complete cds |
| AF010403 | Homo sapiens ALR mRNA, complete cds |
| X12794 | X12794/FEATURE = cds/DEFINITION = HSEAR2 |
| | Human v-erbA related ear-2 gene |
| D42046 | Human mRNA for KIAA0083 gene, partial cds |
| AF000987 | Homo sapiens eIF-1A, Y isoform (EIF1AY) mRNA, complete |
| | cds |
| M58459 | Human ribosomal protein (RPS4Y) isoform mRNA, complete |
| | cds |
| AI208485 | qg36f11.x1 Homo sapiens cDNA, 3 end |
| AF054185 | Homo sapiens proteasome subunit HSPC mRNA, complete |
| | cds |
| J05068 | human transcobalamin I mRNA, complete cds |
| L32137 | Human germline oligomeric matrix protein (COMP) mRNA, |
| | complete cds |
| X83127 | H. sapiens mRNA for voltage gated potassium channels, |
| | beta subunit |
| AL050130 | Homo sapiens mRNA; cDNA DKFZp586H051 (from clone |
| | DKFZp586H051) |
| Z97055 | Human DNA sequence from PAC 388M5 on chromosome 22. |
| | Contains a 60S Ribosomal protein L1 like pseudogene, a |
| | chromosomal protein HMG-17 like gene, a Sulfotransferase |
| | (Sulfokinase) like gene, a putative GS2 like gene, a predicted |
| | CpG island, ESTs and STSs |
| AF034102 | Homo sapiens NBMPR-insensitive nucleoside transporter ei |
| | (ENT2) mRNA, complete cds |
| X16666 | Human HOX2I mRNA from the Hox2 locus |

TABLE 10b
Downregulated genes

| Genbank | Description |
|---|---|
| W80358 | zh49a07.s1 Homo sapiens cDNA, 3 end |
| AF076292 | Homo sapiens TGF-beta |
| X83877 | H. sapiens mRNA for ABP |
| AF083322 | Homo sapiens centriole associated protein CEP110 mRNA, |
| | complete cds |
| Y00064 | Human mRNA for secretogranin I (chromogranin B) |
| L26336 | Human heat shock protein HSPA2 gene, complete cds |
| AB011106 | Homo sapiens mRNA for KIAA0534 protein, partial cds |
| S66213 | integrin alpha 6B [human, mRNA Partial, 528 nt] |
| AF093774 | Homo sapiens type 2 iodothyronine deiodinase mRNA, |
| | complete cds and 3UTR |
| L41607 | Human beta-1,6-N-acetylglucosaminyltransferase (IGnT) |
| | gene |
| Spermidine | Spermidine/Spermine N1-Acetyltransferase, Alt. Splice 2 |
| U43604 | Human unidentified mRNA, partial sequence |
| D00408 | D00408/FEATURE = /DEFINITION = HUMXYPFLA |
| | Human fetal liver cytochrome P-450 (P-450 HFLa), |
| | complete cds |
| S68805 | L-arginine-glycine amidinotransferase [human, kidney |
| | carcinoma cells, mRNA, 2330 nt] |
| AB020665 | Homo sapiens mRNA for KIAA0858 protein, partial cds |
| AB014593 | Homo sapiens mRNA for KIAA0693 protein, partial cds |
| U13045 | Human nuclear respiratory factor-2 subunit beta 1 mRNA, |
| | complete cds |
| J03870 | Human cystatin SA-I mRNA, complete cds |
| U13696 | U13696/FEATURE = cds/DEFINITION = HSU13696 |
| | Human homolog of yeast mutL (hPMS2) gene, |
| | complete cds |
| M86407 | Homo sapiens alpha actinin 3 (ACTN3) mRNA, |
| | complete cds |
| W25945 | 17c5 Homo sapiens cDNA |
| U34962 | Human transcription factor HCSX (hCsx) mRNA, |
| | complete cds |
| AF033382 | Homo sapiens potassium channel mRNA, complete cds |
| U45255 | Human paired-box protein PAX2 (PAX2) gene |
| AA767013 | oa42a08.s1 Homo sapiens cDNA |
| W25951 | 17d10 Homo sapiens cDNA |
| AF071504 | Homo sapiens syntaxin 11 mRNA, complete cds |
| AB011095 | Homo sapiens mRNA for KIAA0523 protein, partial cds |
| M29874 | M29874/FEATURE = /DEFINITION = HUMCYP2BB |
| | Human cytochrome P450-IIB (hIIB1) mRNA, complete cds |
| L08599 | L08599/FEATURE = /DEFINITION = HUMUVOECAD |
| | Human uvomorulin (E-cadherin) (UVO) mRNA, |
| | complete cds |

### Example 11

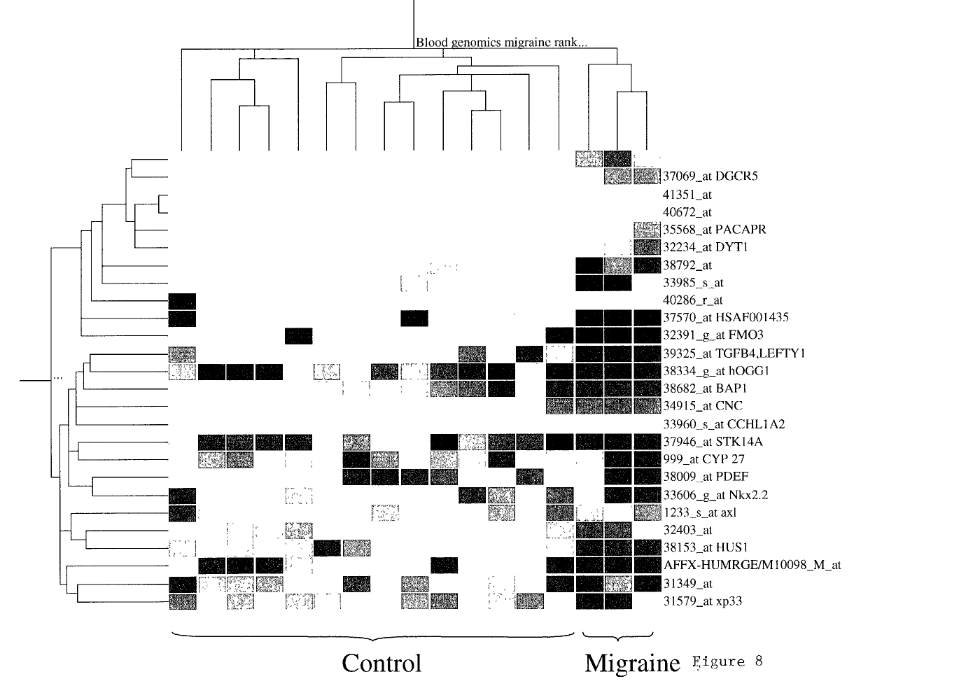

This example demonstrates that the pattern of expression for each individual with acute migraine headaches as compared to individuals without acute migraine headaches. Blood is obtained from individual with acute migraine headaches and individuals without acute migraine headaches. The patterns of expressions are captured and analyzed as described in Example 4. As shown in FIG. 8, there is a defined pattern of expression for individual with acute migraine headaches that is different from individual without acute migraine headaches.

The data below demonstrates the pattern of expression for acute migraine headaches. Table 11a and 11b give lists of genes upregulated or downregulated for acute migraine headaches. This data demonstrates how the pattern of expression in the blood of individuals is unique and can be used to assess headaches, including acute migraine headaches, in an individual.

TABLE 11a
Upregulated genes

| Genbank | Description |
|---|---|
| U81523 | Human endometrial bleeding associated factor mRNA, |
| | complete cds |
| M91368 | Human Na+ |
| Y11731 | H. sapiens mRNA for DNA glycosylase |
| AF045581 | Homo sapiens BRCA1 associated protein 1 (BAP1) mRNA, |
| | complete cds |
| M94172 | Human N-type calcium channel alpha-1 subunit mRNA, |
| | complete cds |
| M60724 | Human p70 ribosomal S6 kinase alpha-I mRNA, complete cds |
| M76125 | M76125/FEATURE = /DEFINITION = HUMTYRKINR |
| | Human tyrosine kinase receptor (axl) mRNA, complete cds |
| AF071538 | Homo sapiens Ets transcription factor PDEF (PDEF) mRNA, |
| | complete cds |
| AF019415 | untitled |
| M10098 | M10098 Human 18S rRNA gene, complete (_5, _M, _3 |
| | represent transcript regions 5 prime, Middle, and 3 prime |
| | respectively) |
| L10403 | Homo sapiens DNA binding protein for surfactant protein B |
| | mRNA, complete cds |
| U86813 | Homo sapiens serotonin-7 receptor pseudogene, |
| | complete sequence |
| AF005082 | Homo sapiens skin-specific protein (xp33) mRNA, partial cds |
| AF076844 | Homo sapiens Hus1-like protein (HUS1) mRNA, |
| | complete cds |
| X59812 | X59812/FEATURE = cds/DEFINITION = HSVD3HYD |
| | H. sapiens CYP 27 mRNA for vitamin D3 25-hydroxylase |

TABLE 11b
Downregulated genes

| Genbank | Description |
|---|---|
| U79115 | U79115/FEATURE = /DEFINITION = HSU79115 |
| | Human death adaptor molecule RAIDD (RAIDD) mRNA, |
| | complete cds |
| X91348 | H. sapiens predicted non coding cDNA (DGCR5) |
| AD001528 | Homo sapiens spermidine aminopropyltransferase mRNA, |
| | complete cds |
| W28616 | 49b9 Homo sapiens cDNA |
| AA885106 | am31h01.s1 Homo sapiens cDNA, 3 end |
| AF001435 | Human clone iota unknown protein mRNA, complete cds |
| AF007871 | Homo sapiens torsinA (DYT1) mRNA, complete cds |
| D17516 | Homo sapiens mRNA for PACAP receptor, complete cds |
| AL050370 | Homo sapiens mRNA; cDNA DKFZp566O0546 (from |
| | clone DKFZp566O0546) |
| AL021026 | dJ127D3.2 (Flavin-containing Monooxygenase family |
| | protein) |
| U57721 | Human L-kynurenine hydrolase mRNA, complete cds |

### Example 12

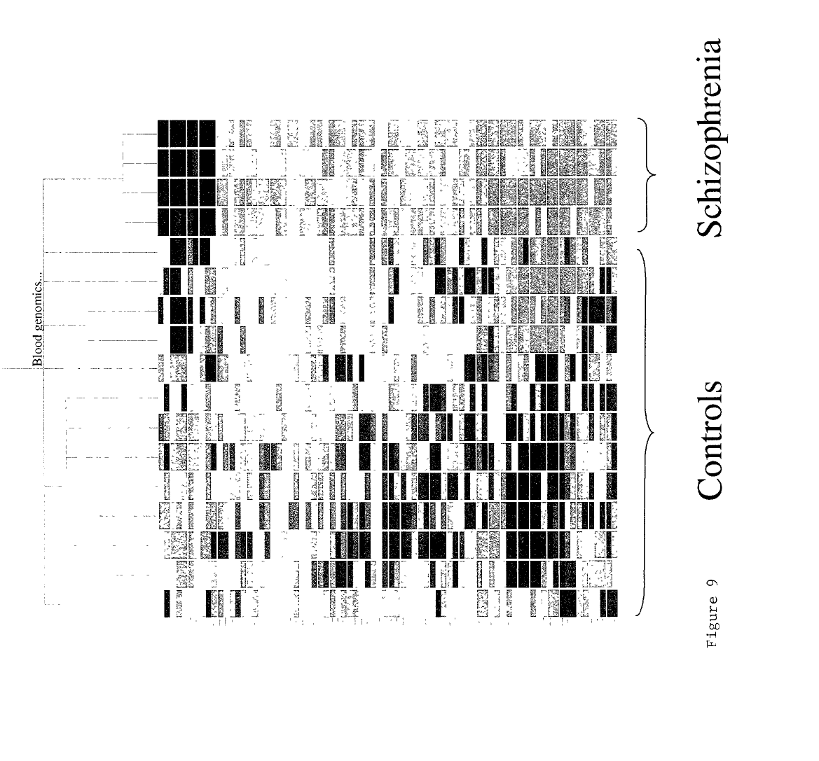

This example demonstrates that the pattern of expression for each individual with schizophrenia as compared to individuals without schizophrenia. Blood is obtained from individual with schizophrenia and individuals without schizophrenia. The patterns of expression are captured and analyzed as described in Example 4. As shown in FIG. 9, there is a defined pattern of expression for individual with schizophrenia that is different from individual without schizophrenia.

The data below demonstrates the pattern of expression for schizophrenia. Table 12a and 12b give lists of genes upregulated or downregulated for schizophrenia. This data demonstrates how the pattern of expression in the blood of individuals is unique and can be used to assess schizophrenia in an individual.

TABLE 12a
Upregulated genes

| Genbank | Description |
|---|---|
| Z54367 | H. sapiens gene for plectin |
| D79989 | Human mRNA for KIAA0167 gene, complete cds |
| AF060865 | Homo sapiens chromosome 16 zinc finger protein ZNF210 |
| | (ZNF210) mRNA, complete cds |
| X69699 | H. sapiens Pax8 mRNA |
| X80907 | X80907/FEATURE = /DEFINITION = HSPHOSINK |
| | H. sapiens mRNA for p85 beta subunit of phosphatidyl- |
| | inositol-3-kinase |
| D45421 | Human mRNA for phosphodiesterase I alpha, complete cds |
| Z83838 | Human DNA sequence from PAC 127B20 on chromosome |
| | 22q11.2-qter, contains gene for GTPase-activating protein |
| | similar to rhoGAP protein. ribosomal protein L6 |
| | pseudogene, ESTs and CA repeat |
| D90239 | Human mRNA for glycine decarboxylase |
| AA203717 | zx52f12.r1 Homo sapiens cDNA, 5 end |
| Z97029 | Homo sapiens mRNA for ribonuclease H I large subunit |

TABLE 12b
Downregulated genes

| Genbank | Description |
|---|---|
| X02956 | X02956/FEATURE = cds/DEFINITION = |
| | HSIFNA5 Human interferon alpha gene IFN- |
| | alpha 5 |
| X97630 | X97630/FEATURE = /DEFINITION = |
| | HSSTPKEMK H. sapiens mRNA for serine/ |
| | threonine protein kinase EMK |
| X75756 | X75756/FEATURE = cds/DEFINITION = |
| | HSPKCMU H. sapiens mRNA for protein kinase C |
| | mu |
| D25303 | Human mRNA for integrin alpha subunit, complete |
| | cds |
| L36033 | Human pre-B cell stimulating factor homologue |
| | (SDF1b) mRNA, complete cds |
| D87440 | Human mRNA for KIAA0252 gene, partial cds |
| M16505 | Human steroid sulfatase (STS) mRNA, complete cds |
| M27533 | Human Ig rearranged B7 protein mRNA VC1- |
| | region, complete cds |
| M81652 | Homo sapiens semenogelin II mRNA, complete cds |
| Z97632 | dJ196E23.3 (bombesin-like receptor 3 (Bombesin |
| | Receptor subtype-3, Uterine Bombesin Receptor, |
| | BRS-3)) |
| AL021026 | dJ127D3.2 (Flavin-containing Monooxygenase |
| | family protein) |
| X91868 | H. sapiens mRNA for SIX1 protein |
| AF056732 | untitled |
| Insulin- | Insulin-Like Growth Factor Ib |
| LikeGrowthFactorIb | |
| S38742 | S38742/FEATURE = /DEFINITION = S38742 |
| | HOX11 = HOX11 homeodomain {homeobox} |
| | [human, mRNA, 1988 nt] |
| AJ010901 | Homo sapiens MUC4 gene, 3 flanking region |
| AA156237 | zl50c09.s1 Homo sapiens cDNA, 3 end |
| U85658 | Human transcription factor ERF-1 mRNA, |
| | complete cds |
| AI820718 | ye38e04.y5 Homo sapiens cDNA, 5 end |
| X58199 | Human mRNA for beta adducin |
| AB007957 | Homo sapiens mRNA, chromosome 1 specific |
| | transcript KIAA0488 |
| AJ001875 | Homo Sapiens mRNA, partial cDNA sequence from |
| | cDNA selection, DCR1-17.0 |
| AI041520 | ov82a04.x1 Homo sapiens cDNA, 3 end |
| Z48054 | H. sapiens mRNA for peroxisomal targeting signal |
| | 1 (SKL type) receptor |
| S81661 | S81661/FEATURE = /DEFINITION = S81661 |
| | Keratinocyte growth factor [human, mRNA, |
| | 1200 nt] |
| X74331 | X74331/FEATURE = cds/DEFINITION = |
| | HSPRIM2 H. sapiens mRNA for DNA primase |
| | (subunit p58) |
| Z93241 | dJ222E13.1a.1 (C-terminal part of novel protein |
| | dJ222E13.1) (partial isoform 1) |
| X12654 | Human mRNA for cell cycle gene ROC1 |
| X80026 | H. sapiens B-cam mRNA |
| D82070 | Human aC1 mRNA, complete cds |
| U04313 | U04313/FEATURE = /DEFINITION = HSU04313 |
| | Human maspin mRNA, complete cds |
| W28846 | 52g2 Homo sapiens cDNA |
| AB023194 | Homo sapiens mRNA for KIAA0977 protein, |
| | complete cds |
| AF070577 | Homo sapiens clone 24461 mRNA sequence |
| W28876 | 52h7 Homo sapiens cDNA |
| AF060503 | Homo sapiens zinc finger protein (ZF5128) mRNA, |
| | complete cds |
| M26856 | M26856/FEATURE = cds/DEFINITION = |
| | HUMCP21OH Human 21-hydroxylase B gene, |
| | complete cds |
| X63380 | Homo sapiens mRNA for serum response factor- |
| | related protein, RSRFC2 |
| M88461 | Human neuropeptide Y peptide YY receptor mRNA, |
| | complete cds |
| W28438 | 47g10 Homo sapiens cDNA |
| W28887 | 53b4 Homo sapiens cDNA |
| D25303 | D25303/FEATURE = /DEFINITION = HUMIAS |
| | Human mRNA for integrin alpha subunit, |
| | complete cds |
| AF065314 | Homo sapiens cone photoreceptor cGMP-gated |
| | channel alpha subunit (CNGA3) mRNA, complete |
| | cds |
| AF100780 | Homo sapiens connective tissue growth factor |
| | related protein WISP-2 (WISP2) mRNA, complete |
| | cds |
| AI824126 | wj46e05.x1 Homo sapiens cDNA, 3 end |
| L36069 | Human high conductance inward rectifier potassium |
| | channel alpha subunit mRNA, complete cds |
| D16626 | Human mRNA for histidase, complete cds |
| L20316 | Human glucagon receptor mRNA, complete cds |
| AF076292 | Homo sapiens TGF-beta |
| AL109707 | Homo sapiens mRNA full length insert cDNA |
| | clone EUROIMAGE 295344 |
| M31525 | Human MHC class II lymphocyte antigen |
| | (HLA-DNA) gene, complete cds |
| Y13620 | Y13620/FEATURE = /DEFINITION = |
| | HSRNABCL9 Homo sapiens mRNA for BCL9 gene |
| AB014520 | Homo sapiens mRNA for KIAA0620 protein, |
| | partial cds |
| W80358 | zh49a07.s1 Homo sapiens cDNA, 3 end |
| W25951 | 17d10 Homo sapiens cDNA |
| S62138 | TLS |
| X15573 | Human liver-type 1-phosphofructokinase (PFKL) |
| | mRNA, complete cds |
| AL049261 | Homo sapiens mRNA; cDNA DKFZp564E053 |
| | (from clone DKFZp564E053) |
| M16276 | Human MHC class II HLA-DR2-Dw12 mRNA |
| | DQw1-beta, complete cds |
| M29874 | M29874/FEATURE = /DEFINITION = |
| | HUMCYP2BB Human cytochrome P450-IIB hIIB1) |
| | mRNA, complete cds |
| AF050078 | untitled |
| AI394290 | tg09f06.x1 Homo sapiens cDNA, 3 end |
| AF004841 | Homo sapiens CDO mRNA, complete cds |
| D23673 | Human mRNA, clone HH109 (screened by the |
| | monoclonal antibody of insulin receptor |
| | substrate-1 (IRS-1)) |
| AJ132445 | Homo sapiens CLDN14 gene |
| Z11584 | H. sapiens mRNA for NuMA protein |
| AC002398 | Human DNA from chromosome 19-specific cosmid |
| | F25965, genomic sequence |

### Example 13

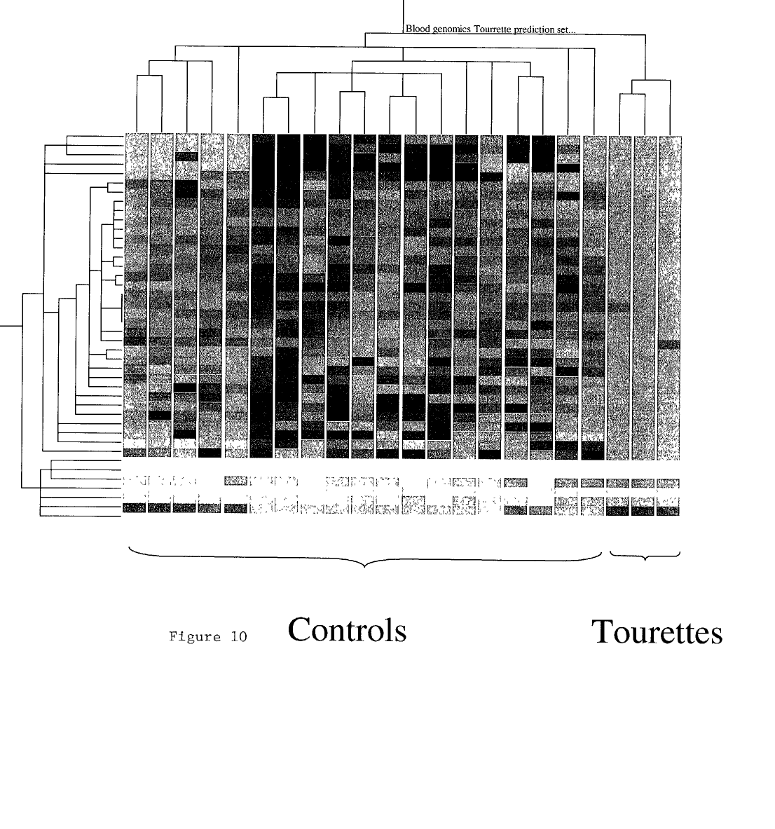

This example demonstrates that the pattern of expression for each individual with Tourettes as compared to individuals without Tourettes. Blood is obtained from individual with Tourettes and individuals without Tourettes. The patterns of expressions are captured and analyzed as described in Example 4. As shown in FIG. 10, there is a defined pattern of expression for individual with Tourettes that is different from individual without Tourettes.

The data below demonstrates the pattern of expression for Tourettes. Table 13a and 13b give lists of genes upregulated or downregulated for Tourettes. This data demonstrates how the pattern of expression in the blood of individuals is unique and can be used to assess Tourettes in an individual.

TABLE 13a
Upregulated genes

| Genbank | Description |
|---|---|
| AI218431 | qh24d10.x1 Homo sapiens cDNA, 3 end |
| AW043925 | wy82b07.x1 Homo sapiens cDNA, 3 end |
| Y17673 | Homo sapiens mRNA for nebulette, incomplete splice |
| | variant, partial |
| X07495 | Human mRNA for cp19 homeobox from HOX-3 locus |
| W27997 | 43e3 Homo sapiens cDNA |
| AI347129 | tc04a03.x1 Homo sapiens cDNA, 3 end |
| U39576 | Human butyrophilin precursor mRNA, complete cds |
| AF051160 | Homo sapiens tyrosine phosphatase (PRL-1) gene, |
| | complete cds |
| U77968 | Human neuronal PAS1 (NPAS1) mRNA, complete cds |
| AJ132337 | Homo sapiens mRNA for chemokine receptor CCR9 |
| U07620 | U07620/FEATURE = /DEFINITION = HSU07620 Human |
| | MAP kinase mRNA, complete cds |

TABLE 13b
Downregulated genes

| Genbank | Description |
|---|---|
| X54637 | X54637/FEATURE = cds/DEFINITION = HSTYK2 |
| | Human tyk2 mRNA for non-receptor protein tyrosine |
| | kinase |
| U53204 | Human plectin (PLEC1) mRNA, complete cds |
| AB014587 | Homo sapiens mRNA for KIAA0687 protein, partial cds |
| U31525 | Human glycogenin mRNA, complete cds |
| D38251 | Homo sapiens mRNA for RPB5 (XAP4), complete cds |
| D14663 | Human mRNA for KIAA0107 gene, complete cds |
| J05448 | J05448/FEATURE = /DEFINITION = HUMRPOLAA |
| | Human RNA polymerase subunit hRPB 33, mRNA |
| X52773 | X52773/FEATURE = cds/DEFINITION = HSRARLP |
| | Human mRNA for retinoic acid receptor like protein |
| U52840 | Homo sapiens semaphorin F homolog mRNA, complete |
| | cds |
| AB002311 | Human mRNA for KIAA0313 gene, complete cds |
| AI796048 | wh41g06.x1 Homo sapiens cDNA, 3 end |
| D10202 | D10202/FEATURE = /DEFINITION = HUMPAFRE |
| | Homo sapiens mRNA for platelet-activating factor |
| | receptor, complete cds |
| U22055 | Human 100 kDa coactivator mRNA, complete cds |
| U73704 | Homo sapiens 48 kDa FKBP-associated protein FAP48 |
| | mRNA, complete cds |
| L13291 | Human ADP-ribosylarginine hydrolase mRNA, complete |
| | cds |
| AF067139 | Homo sapiens NADH-ubiquinone oxidoreductase |
| | NDUFS3 subunit mRNA, nuclear gene encoding |
| | mitochondrial protein, complete cds |
| AF038203 | Homo sapiens clone 23596 mRNA sequence |
| AB023181 | Homo sapiens mRNA for KIAA0964 protein, complete |
| | cds |
| AI864120 | wg64a06.x1 Homo sapiens cDNA, 3 end |
| AC002544 | Homo sapiens Chromosome 16 BAC clone |
| | CIT987SK-A-761H5 |
| X75621 | Homo sapiens TSC2 mRNA for tuberin |
| M30938 | M30938/FEATURE = mRNA#2/DEFINITION = |
| | HUMKUP Human Ku (p70/p80) subunit mRNA, |
| | complete cds |
| AI417075 | tg78e09.x1 Homo sapiens cDNA, 3 end |
| AL035447 | Human DNA sequence from clone 1183I21 on |
| | chromosome 20q12. Contains a novel gene and the first |
| | exon of a putative novel gene for a protein similar to |
| | predicted fly and worm proteins. Contains ESTs, STSs, |
| | GSSs and a putative CpG island |
| U72936 | U72936/FEATURE = /DEFINITION = HSU72936 |
| | Homo sapiens putative DNA dependent ATPase and |
| | helicase (ATRX) mRNA, alternatively spliced product 1, |
| | complete cds |
| U08997 | Human glutamate dehydrogenase gene, complete cds |
| AF055479 | Homo sapiens lung cancer candidate FUS1 (FUS1) |
| | mRNA, complete cds |
| AF070523 | Homo sapiens JWA protein mRNA, complete cds |
| M11058 | Human 3-hydroxy-3-methylglutaryl coenzyme A |
| | reductase mRNA, complete cds |
| U19969 | Human two-handed zinc finger protein ZEB mRNA, |
| | partial cds |
| X02344 | Homo sapiens beta 2 gene |
| D34625 | Human TBXAS1 gene for thromboxane synthase, |
| | promoter region and |
| M60721 | M60721/FEATURE = mRNA/DEFINITION = |
| | HUMHB24 Human homeobox gene, complete cds |
| X76488 | H. sapiens mRNA for lysosomal acid lipase |
| AL031781 | dJ51J12.1.3 (human ortholog of mouse KH Domain |
| | RNA Binding protein QKI-7 (isoform 3) |
| U82939 | Homo sapiens p53 binding protein mRNA, complete cds |
| U96074 | Human translation initiation factor eIF3 p44 subunit |
| | mRNA, complete cds |
| X65784 | H. sapiens CAR gene |
| W30677 | zb75h10.r1 Homo sapiens cDNA, 5 end |
| U47077 | Human DNA-dependent protein kinase catalytic subunit |
| | (DNA-PKcs) mRNA, complete cds |
| M32373 | Human arylsulfatase B (ASB) mRNA, complete cds |
| M34175 | Human beta adaptin mRNA, complete cds |
| U90313 | U90313/FEATURE = /DEFINITION = HSU90313 |
| | Human glutathione-S-transferase homolog mRNA, |
| | complete cds |
| AI683748 | tw53e07.x1 Homo sapiens cDNA, 3 end |
| AB014603 | Homo sapiens mRNA for KIAA0703 protein, complete |
| | cds |
| AF089814 | Homo sapiens growth suppressor related (DOC-1R) |
| | mRNA, complete cds |
| AB007960 | chromosome 1 specific transcript KIAA0491 |
| M28393 | Human perform mRNA, complete cds |
| X84709 | H. sapiens mRNA for mediator of receptor-induced |
| | toxicity |
| AB014536 | Homo sapiens mRNA for KIAA0636 protein, complete |
| | cds |
| L36870 | Homo sapiens MAP kinase kinase 4 (MKK4) mRNA, |
| | complete cds |
| ALO80144 | Homo sapiens mRNA; cDNA DKFZp434N093 (from |
| | clone DKFZp434N093) |
| Z78324 | HSZ78324 Homo sapiens cDNA |
| AF052111 | Homo sapiens clone 23953 mRNA sequence |
| AB002354 | Human mRNA for KIAA0356 gene, complete cds |
| AI436567 | ti03b09.x1 Homo sapiens cDNA, 3 end |
| AF042385 | Homo sapiens cyclophilin-33A (CYP-33) mRNA, |
| | complete cds |
| Z25821 | H. sapiens gene for mitochondrial dodecenoyl-CoA |
| | delta-isomerase, exons 1 and 2 |
| U94778 | Human PEST phosphatase interacting protein homolog |
| | (H-PIP) mRNA, complete cds |
| L13435 | Human chromosome 3p21.1 gene sequence |
| M22898 | M22898/FEATURE = mRNA/DEFINITION = |
| | HUMP53A11 Human phosphoprotein p53 gene exon 11 |
| J05070 | Human type IV collagenase mRNA, complete cds |
| U47634 | U47634/FEATURE = /DEFINITION = HSU47634 |
| | Human beta-tubulin class III isotype (beta-3) mRNA, |
| | complete cds |
| X99906 | Homo sapiens mRNA for alpha endosulfine |
| AF051850 | Homo sapiens supervillin mRNA, complete cds |
| AC002400 | Human Chromosome 16 BAC clone |
| | CIT987SK-A-735G6 |
| AB028951 | Homo sapiens mRNA for KIAA1028 protein, partial cds |
| Y09538 | H. sapiens mRNA for ZNF185 gene |
| AF041259 | Homo sapiens breast cancer putative transcription factor |
| | (ZABC1) mRNA, complete cds |
| L13972 | Homo sapiens beta-galactoside alpha-2,3-sialyltransferase |
| | (SIAT4A) mRNA, complete cds |
| X87344 | H. sapiens DMA, DMB, HLA-Z1, IPP2, LMP2, TAP1, |
| | LMP7, TAP2, DOB, DOB2 and RING8 9, 13 and 14 |
| | genes |
| W28299 | 44h4 Homo sapiens cDNA |
| X53390 | Human mRNA for upstream binding factor (hUBF) |
| AI189287 | qd05c04.x1 Homo sapiens cDNA, 3 end |
| L34587 | L34587/FEATURE = /DEFINITION = HUMRPIE |
| | Homo sapiens RNA polymerase II elongation factor |
| | SIII, p15 subunit mRNA, complete cds |
| D13146 | D13146/FEATURE = mRNA#1/DEFINITION = |
| | HUM3CNP3 Homo sapiens gene for 2,3-cyclic- |
| | nucleotide 3-phosphodiesterase, exon 3 and complete cds |
| AB018348 | Homo sapiens mRNA for KIAA0805 protein, partial cds |
| AF052155 | Homo sapiens clone 24761 mRNA sequence |
| S74017 | S74017/FEATURE = /DEFINITION = S74017 Nrf = |
| | 2NF-E2-like basic leucine zipper transcriptional activator |
| | [human, hemin-induced K562 cells, mRNA, 2304 nt] |
| D87127 | D87127/FEATURE = /DEFINITION = D87127 Homo |
| | sapiens mRNA for translocation protein-1, complete cds |
| U70063 | U70063/FEATURE = /DEFINITION = HSU70063 |
| | Human acid ceramidase mRNA, complete cds |
| Tubulin, Beta2 | Tubulin, Beta 2 |
| AF075599 | Homo sapiens ubiquitin conjugating enzyme 12 (UBC12) |
| | mRNA, complete cds |
| U80184 | Homo sapiens FLII gene, complete cds |
| U89505 | Human Hlark mRNA, complete cds |
| AF031647 | Homo sapiens JAB1-containing signalosome subunit 3 |
| | (SGN3) mRNA, complete cds |
| D83664 | Human mRNA for CAAF1 (calcium-binding protein in |
| | amniotic fluid 1), complete cds |
| AA457029 | aa38b10.s1 Homo sapiens cDNA, 3 end |
| AL044599 | DKFZp434N192_s1 Homo sapiens cDNA, 3 end |
| X06409 | Human mRNA fragment for activated c-raf-1 (exons |
| | 8-17) |
| Proteinkinase | Protein Kinase Ht31, Camp-Dependent |
| Ht31, Camp- | |
| Dependent | |
| U79270 | Human clone 23707 mRNA, partial cds |
| AF097358 | Homo sapiens mast cell function-associated antigen |
| | homolog (MAFA) mRNA, complete cds |
| Glucocorticoid | Glucocorticoid Receptor, Beta |
| Receptor, Beta | |
| M68864 | Human ORF mRNA, complete cds |
| U15655 | Human ets domain protein ERF mRNA, complete cds |
| Y00281 | Human mRNA for ribophorin I |
| X95762 | H. sapiens mRNA for aminopeptidase P-like |
| U83115 | Human non-lens beta gamma-crystallin like protein |
| | (AIM1) mRNA, partial cds |
| D87450 | Human mRNA for KIAA0261 gene, partial cds |
| U17989 | Homo sapiens nuclear autoantigen GS2NA mRNA, |
| | complete cds |
| D26535 | Human gene for dihydrolipoamide succinyltransferase, |
| | complete cds (exon 1-15) |
| D12686 | D12686/FEATURE = /DEFINITION = HUMEIF4G |
| | Human mRNA for eukaryotic initiation factor 4 gamma |
| | (eIF-4 gamma) |
| AF098799 | Homo sapiens RanBP7 |
| U18334 | U18334/FEATURE = cds/DEFINITION = |
| | HSUNOSIIC1 Human nitric oxide synthase II |
| | (NOSIIc) gene, partial exon 23 |
| D87444 | Human mRNA for KIAA0255 gene, complete cds |
| AA576724 | nm81b04.s1 Homo sapiens cDNA, 3 end |
| U79282 | Human clone 23801 mRNA sequence |
| AL050369 | Homo sapiens mRNA; cDNA DKFZp566J153 (from |
| | clone DKFZp566J153) |
| D13540 | D13540/FEATURE = /DEFINITION = HUMSHPTP3 |
| | Homo sapiens SH-PTP3 mRNA for protein-tyrosine |
| | phosphatase, complete cds |
| X12433 | Human pHS1-2 mRNA with ORF homologous to |
| | membrane receptor proteins |
| AB028948 | Homo sapiens mRNA for KIAA1025 protein, partial cds |
| D12620 | D12620/FEATURE = /DEFINITION = HUMCYT1 |
| | Homo sapiens mRNA for cytochrome P-450LTBV, |
| | complete cds |
| X91504 | H. sapiens mRNA for ARP1 protein |
| W16505 | zb05e12.r1 Homo sapiens cDNA, 5 end |
| D29677 | Human mRNA for KIAA0054 gene, complete cds |
| AI540318 | tq34f03.x1 Homo sapiens cDNA, 3 end |
| S69189 | peroxisomal acyl-coenzyme A oxidase [human, liver, |
| | mRNA, 3086 nt[ |
| AB003177 | AB003177/FEATURE = /DEFINITION = AB003177 |
| | Homo sapiens mRNA for proteasome subunit p27, |
| | complete cds |
| Z84718 | Z84718/FEATURE = cds#5/DEFINITION = HS322B1 |
| | Human DNA sequence from clone 322B1 on |
| | chromosome 22q11-12, complete sequence |
| | [Homo sapiens] |
| AW005997 | wz91c01.x1 Homo sapiens cDNA, 3 end |
| AJ237839 | Homo sapiens mRNA for hypothetical protein |
| U82277 | Human immunoglobulin-like transcript 1b mRNA, |
| | complete cds |
| S46950 | adenosine A2 receptor [human, hippocampal, mRNA, |
| | 2572 nt] |
| AA478904 | zv20c05.r1 Homo sapiens cDNA, 5 end |
| X71440 | H. sapiens mRNA for peroxisomal acyl-CoA oxidase |
| AI557064 | PT2.1_13_A12.r Homo sapiens cDNA, 3 end |
| AB006202 | Homo sapiens mRNA for cytochrome b small subunit |
| | of complex II, complete cds |
| AD000092 | AD000092/FEATURE = cds#2/DEFINITION = |
| | CH19HHR23 Homo sapiens DNA from chromosome |
| | 19p13.2 cosmids R31240, R30272 and R28549 |
| | containing the EKLF, GCDH, CRTC, and RAD23A |
| | genes, genomic sequence |
| | X85545/FEATURE = cds/DEFINITION = HSPKX1MR |
| | H. sapiens mRNA for protein kinase, PKX1/NOTE = |
| | replacement of probe set 132_at |
| AF047185 | Homo sapiens NADH-ubiquinone oxidoreductase subunit |
| | CI-B8 mRNA, complete cds |
| AF104421 | Homo sapiens isolate normal patient 1 uroporphyrinogen |
| | decarboxylase (UROD) mRNA, complete cds |
| X98253 | H. sapiens ZNF183 gene |
| Ubiquitin- | Ubiquitin-Conjugating Enzyme Ubch5 |
| Conjugating- | |
| EnzymeUbch5 | |
| AI670788 | tz10c02.x1 Homo sapiens cDNA, 3 end |
| AB017551 | Homo sapiens mRNA for 16G2, complete cds |
| M80359 | Human protein p78 mRNA, complete cds |
| U26710 | Human cbl-b mRNA, complete cds |
| U27460 | Human uridine diphosphoglucose pyrophosphorylase |
| | mRNA, complete cds |
| AI347155 | tc04c11.x1 Homo sapiens cDNA, 3 end |
| AL023657 | Homo sapiens SH2D1A cDNA, formerly known |
| | as DSHP |
| AF038564 | Homo sapiens atrophin-1 interacting protein 4 (AIP4) |
| | mRNA, partial cds |
| Y07604 | H. sapiens mRNA for nucleoside-diphosphate kinase |
| U76247 | Human hSIAH1 mRNA, complete cds |
| M96803 | Human general beta-spectrin (SPTBN1) mRNA, |
| | complete cds |
| Z69043 | H. sapiens mRNA translocon-associated protein delta |
| | subunit precursor |
| U07158 | Human syntaxin mRNA, complete cds |
| AL078641 | Human DNA sequence from clone 494G10 on |
| | chromosome 22 Contains part of a gene similar to |
| | phorbolin 2, ESTs and a GSS |
| M29551 | Human calcineurin A2 mRNA, complete cds |
| AF042083 | Homo sapiens BH3 interacting domain death agonist |
| | (BID) mRNA, complete cds |
| L32977 | Homo sapiens (clone f17252) ubiquinol cytochrome c |
| | reductase Rieske iron-sulphur protein (UQCRFS1) gene |
| AF059681 | Homo sapiens serine |
| M76231 | Human sepiapterin reductase mRNA, complete cds |
| AL031427 | dJ167A19.3 (novel protein) |
| AI935146 | wp14b12.x1 Homo sapiens cDNA, 3 end |
| AF093771 | Homo sapiens mitoxantrone resistance protein 1 mRNA, |
| | partial sequence |
| U79267 | Human clone 23840 mRNA, partial cds |
| M28439 | M28439/FEATURE = cds/DEFINITION = |
| | HUMKER16A8 Human keratin type 16 gene, exon 8 |
| AF000364 | Homo sapiens heterogeneous nuclear ribonucleoprotein R |
| | mRNA, complete cds |
| D82351 | Human retropseudogene MSSP-1 DNA, complete cds |
| M28212 | M28212/FEATURE = /DEFINITION = HUMRAB6A |
| | Homo sapiens GTP-binding protein (RAB6) mRNA, |
| | complete cds |
| AJ236885 | Homo sapiens mRNA for ZBP-89 protein |
| U79291 | Human clone 23721 mRNA sequence |
| AF015926 | Homo sapiens ezrin-radixin-moesin binding |
| | phosphoprotein-50 mRNA, complete cds |
| AL050087 | Homo sapiens mRNA; cDNA DKFZp434O031 (from |
| | clone DKFZp434O031) |
| AF038952 | Homo sapiens cofactor A protein mRNA, complete cds |
| AC002073 | Human PAC clone DJ515N1 from 22q11.2-q22 |
| L15388 | L15388/FEATURE = /DEFINITION = HUMGRK5A |
| | Human G protein-coupled receptor kinase (GRK5) |
| | mRNA, complete cds |
| L23134 | Homo sapiens metase (MET-1) mRNA, complete cds |
| D42087 | Human mRNA for KIAA0118 gene, partial cds |
| AL049324 | Homo sapiens mRNA; cDNA DKFZp564D246 (from |
| | clone DKFZp564D246) |
| U63717 | U63717/FEATURE = /DEFINITION = HSU63717 |
| | Homo sapiens osteoclast stimulating factor mRNA, |
| | complete cds |
| AB011113 | Homo sapiens mRNA for KIAA0541 protein, partial cds |
| D00860 | Homo sapiens mRNA for phosphoribosyl pyrophosphate |
| | synthetase subunit I, complete cds |
| D82348 | Homo sapiens mRNA for 5-aminoimidazole-4- |
| | carboxamide-1-beta-D-ribon ucleotide transformylase |
| D31766 | Human mRNA for KIAA0060 gene, complete cds |
| L13858 | Human guanine nucleotide exchange factor mRNA, |
| | complete cds |
| AA151716 | zo30d07.s1 Homo sapiens cDNA, 3 end |
| AF019083 | Homo sapiens phosphatase and tensin homolog 2 (PTH2) |
| | mRNA, partial cds |
| AF017445 | Homo sapiens GDP-L-fucose pyrophosphorylase (GFPP) |
| | mRNA, complete cds |
| AF038186 | Homo sapiens clone 23914 mRNA sequence |
| AB018257 | Homo sapiens mRNA for KIAA0714 protein, partial cds |
| AF049891 | Homo sapiens tyrosylprotein sulfotransferase-2 mRNA, |
| | complete cds |
| AF052186 | Homo sapiens clone 24431 mRNA sequence |
| AF070582 | Homo sapiens clone 24766 mRNA sequence |
| AF055020 | Homo sapiens clone 24722 unknown mRNA, partial cds |
| AF052138 | Homo sapiens clone 23718 mRNA sequence |
| AB000468 | Homo sapiens mRNA for zinc finger protein, complete |
| | cds, clone-RES4-26 |
| M31158 | Human cAMP-dependent protein kinase subunit RII-beta |
| | mRNA, complete cds |
| AB002360 | Human mRNA for KIAA0362 gene, partial cds |
| AB018285 | Homo sapiens mRNA for KIAA0742 protein, partial cds |
| AF013759 | Homo sapiens calumein (Calu) mRNA, complete cds |
| D87292 | Homo sapiens mRNA for rhodanese, complete cds |
| AB023143 | Homo sapiens mRNA for KIAA0926 protein, |
| | complete cds |
| AA194159 | zr37h01.r1 Homo sapiens cDNA, 5 end |
| M96824 | Human nucleobindin precursor mRNA, complete cds |
| X78925 | H. sapiens HZF2 mRNA for zinc finger protein |
| D25235 | Human mRNA for alpha1C adrenergic receptor, |
| | complete cds |
| M62896 | Human lipocortin (LIP) 2 pseudogene mRNA, complete |
| | cds-like region |
| AB000712 | Homo sapiens hCPE-R mRNA for CPE-receptor, |
| | complete cds |
| U26648 | Homo sapiens syntaxin 5 mRNA, complete cds |
| M99439 | Human transducin-like enhancer protein (TLE4) mRNA, |
| | 3 end |
| L42450 | Homo sapiens pyruvate dehydrogenase kinase isoenzyme |
| | 1 (PDK1) mRNA, complete cds |
| AA913812 | ol39a08.s1 Homo sapiens cDNA, 3 end |
| U29185 | Homo sapiens prion protein (PrP) gene, complete cds |
| Y14768 | Homo sapiens DNA, cosmid clones TN62 and TN82 |
| L20321 | L20321/FEATURE = /DEFINITION = HUMSTK2A |
| | Human protein serine/threonine kinase stk2 mRNA, |
| | complete cds |
| M28130 | M28130/FEATURE = mRNA/DEFINITION = |
| | HUMIL8A Human interleukin 8 (IL8) gene, complete |
| | cds |
| AB018312 | Homo sapiens mRNA for KIAA0769 protein, |
| | complete cds |
| U56833 | U56833/FEATURE = /DEFINITION = HSU56833 |
| | Human VHL binding protein-1 (VBP-1) mRNA, |
| | partial cds |
| U59435 | Human cell cycle protein p38-2G4 homolog (hG4-1) |
| | mRNA, complete cds |
| AB018319 | Homo sapiens mRNA for KIAA0776 protein, partial cds |
| AB002381 | Human mRNA for KIAA0383 gene, partial cds |
| M22632 | Human mitochondrial aspartate aminotransferase mRNA, |
| | complete cds |
| AA521060 | aa71e09.s1 Homo sapiens cDNA, 3 end |
| AB015051 | Homo sapiens mRNA for Daxx, complete cds |
| Y07846 | H. sapiens mRNA for GAR22 protein |
| AF023612 | Homo sapiens Dim1p homolog mRNA, complete cds |
| D31883 | Human mRNA for KIAA0059 gene, complete cds |
| U89896 | Homo sapiens casein kinase I gamma 2 mRNA, |
| | complete cds |
| X15949 | X15949/FEATURE = cds/DEFINITION = HSIRF2 |
| | Human mRNA for interferon regulatory factor-2 (IRF-2) |
| AB028980 | Homo sapiens mRNA for KIAA1057 protein, partial cds |
| L42324 | L42324/FEATURE = cds/DEFINITION = HUMFRCG |
| | Homo sapiens (clone GPCR W) G protein-linked |
| | receptor gene (GPCR) gene, 5 end of cds |
| AB023229 | Homo sapiens mRNA for KIAA1012 protein, |
| | complete cds |
| AB020636 | Homo sapiens mRNA for KIAA0829 protein, partial cds |
| D86970 | Human mRNA for KIAA0216 gene, complete cds |
| U01923 | Human BTK region clone ftp-3 mRNA |
| U51007 | Human 26S protease subunit S5a mRNA, complete cds |
| M25322 | Human granule membrane protein-140 mRNA, |
| | complete cds |
| S76638 | S76638/FEATURE = /DEFINITION = S76638 |
| | p50-NF-kappa B homolog [human, peripheral blood T |
| | cells, mRNA, 3113 nt] |
| U60325 | U60325/FEATURE = /DEFINITION = HSU60325 |
| | Human DNA polymerase gamma mRNA, nuclear gene |
| | encoding mitochondrial protein, complete cds |
| U91316 | Human acyl-CoA thioester hydrolase mRNA, |
| | complete cds |
| L08069 | L08069/FEATURE = /DEFINITION = HUMDNAJHOM |
| | Human heat shock protein, E. coli DnaJ homologue |
| | mRNA, complete cds |
| S63912 | D10S102 = FBRNP [human, fetal brain, mRNA, |
| | 3043 nt] |
| D86062 | Human mRNA for KNP-lb, complete cds |
| M98343 | Homo sapiens amplaxin (EMS1) mRNA, complete cds |
| D13315 | Human mRNA for lactoyl glutathione lyase |
| AB018276 | Homo sapiens mRNA for KIAA0733 protein, partial cds |
| X75346 | X75346/FEATURE = cds/DEFINITION = HSMAPKAP |
| | H. sapiens mRNA for MAP kinase activated protein |
| | kinase |
| M28215 | Homo sapiens GTP-binding protein (RABS) mRNA, |
| | complete cds |
| M60784 | Human U1 snRNP-specific protein A gene |
| AB007900 | Homo sapiens KIAA0440 mRNA, partial cds |
| U91512 | Human adhesion molecule ninjurin mRNA, complete cds |
| AF000982 | Homo sapiens dead box, X isoform (DBX) mRNA, |
| | alternative transcript 2, complete cds |
| M12267 | Human ornithine aminotransferase mRNA, complete cds |
| D11094 | Human mRNA for MSS1, complete cds |
| U79260 | Human clone 23745 mRNA, complete cds |
| X55079 | Human lysosomal alpha-glucosidase gene exon 1 |
| D83782 | Human mRNA for KIAA0199 gene, partial cds |
| R38263 | yc92c11.s1 Homo sapiens cDNA, 3 end |
| M12125 | Human fibroblast muscle-type tropomyosin mRNA, |
| | complete cds |
| AB007869 | Homo sapiens KIAA0409 mRNA, partial cds |
| U82130 | U82130/FEATURE = /DEFINITION = HSU82130 |
| | Human tumor susceptiblity protein (TSG101) mRNA, |
| | complete cds |
| U40763 | Human Clk-associated RS cyclophilin CARS-Cyp |
| | mRNA, complete cds |
| W94101 | ze11c11.r1 Homo sapiens cDNA, 5 end |
| AA877795 | nr10g08.s1 Homo sapiens cDNA, 3 end |
| AL049442 | Homo sapiens mRNA; cDNA DKFZp586N1720 (from |
| | clone DKFZp586N1720) |
| AJ223183 | Homo sapiens mRNA for DORA protein |
| X53587 | X53587/FEATURE = mRNA/DEFINITION = |
| | HSINTB4R Human mRNA for integrin beta 4 |
| X99720 | H. sapiens TPRC gene |
| AL050282 | Homo sapiens mRNA; cDNA DKFZp586H2219 (from |
| | clone DKFZp586H2219) |
| AA135683 | zl10c08.r1 Homo sapiens cDNA, 5 end |
| AB002369 | Human mRNA for KIAA0371 gene, complete cds |
| AB014562 | Homo sapiens mRNA for KIAA0662 protein, partial cds |
| AA928996 | oo27f06.s1 Homo sapiens cDNA, 3 end |
| AJ132917 | Homo sapiens mRNA for methyl-CpG-binding protein 2 |
| W27419 | 31a10 Homo sapiens cDNA |
| AL009179 | dJ97D16.6 (Histone H3.1) |
| AF004430 | Homo sapiens hD54 + ins2 isoform (hD54) mRNA, |
| | complete cds |
| D13627 | Human mRNA for KIAA0002 gene, complete cds |
| D78514 | D78514/FEATURE = cds/DEFINITION = D78514 |
| | Homo sapiens mRNA for ubiquitin-conjugating |
| | enzyme, complete cds |
| D14812 | Human mRNA for KIAA0026 gene, complete cds |
| H15872 | ym22b12.r1 Homo sapiens cDNA, 5 end |
| U84971 | Homo sapiens fetal unknown mRNA, complete cds |
| AF040707 | Homo sapiens candidate tumor suppressor gene 21 |
| | protein isoform I mRNA, complete cds |
| AL009179 | dJ97D16.4 (Histone H2B) |
| U05875 | Human clone pSK1 interferon gamma receptor accessory |
| | factor-1 (AF-1) mRNA, complete cds |
| AC004262 | Homo sapiens chromosome 19, cosmid R29368 |
| X77909 | H. sapiens IKBL mRNA |
| D89678 | Homo sapiens mRNA for A + U-rich element RNA |
| | binding factor, complete cds |
| AF070533 | Homo sapiens clone 24619 mRNA sequence |
| X04412 | Human mRNA for plasma gelsolin |
| U37547 | Human IAP homolog B (MIHB) mRNA, complete cds |
| AL050157 | Homo sapiens mRNA; cDNA DKFZp586O0120 (from |
| | clone DKFZp586O0120) |
| U09825 | Human acid finger protein mRNA, complete cds |

The specific embodiments and examples set forth above are provided for illustrative purposes only and are not intended to limit the scope of the following claims. Additional embodiments of the invention and advantages provided thereby will be apparent to one of ordinary skill in the art and are within the scope of the claims.

What is claimed is:

1. A method of injury assessment in an individual comprising the steps of:
a. determining a pattern of expression exhibited by blood cells obtained from the individual and
b. comparing the pattern of expression exhibited by the obtained blood cells to an injury database to assess the injury.

2. A method according to claim 1, wherein the injury is a result of a cause selected from the group comprising cell death, cell dysfunction, genetic abnormalities, or combinations thereof.

3. A method according to claim 1, wherein the pattern of expression comprises patterns of gene expression, protein expression, or combinations thereof.

4. A method according to claim 1, wherein the injury database comprises genomic injury databases, proteomic injury databases, or combinations thereof.

5. A method according to claim 1, wherein the blood cells are obtained from a peripheral blood sample or an organ.

6. A method according to claim 1, wherein the step of determining a pattern of expression exhibited by the obtained blood cells comprises capturing a pattern of expression from the obtained blood cells and defining the pattern of expression.

7. A method according to claim 6, wherein capturing a pattern of expression comprises:
i. isolating RNA or protein from the obtained blood cells,
ii. preparing a probe using the isolated RNA or protein,
iii. applying the probe to a microarray, DNA, RNA, or protein; and
iv. measuring the level of the RNA, protein, or combinations thereof.

8. A method according to claim 6, wherein defining the pattern of expression comprises using an expression method.

9. A method according to claim 6, wherein the step of determining a pattern of expression further comprises ranking the molecules of the captured pattern of expression.

10. A method according to claim 7, wherein the step of preparing a probe using the RNA comprises preparing cDNA or cRNA and labeling the cDNA or cRNA.

11. A method according to claim 9, wherein the expression method comprises statistical analysis, class prediction, clustering, computer programs, or combinations thereof.

12. A method according to claim 3, wherein the genes or proteins in the pattern of gene expression or protein expression comprise intermediate metabolism, immune-related molecules, cytokines, chemokines, immediate early genes, structural genes, neurotransmitters, receptors, signaling molecules, oncogenes, proto-oncogenes, heat shock genes, stress genes, transporters, trophic factors, growth factors, cell cycle genes, lipid metabolism, arachidonic acid metabolism, free radicals, free radical scavengers, metal binding, transporting genes or combinations thereof.

13. A method according to claim 12, wherein the genes in the pattern of gene expression comprise acidosis-induced genes, hypoxia-induced genes, glucose-induced genes, ischemia-induced genes, genes as recited in Table 1, or combinations thereof.

14. A method according to claim 13, wherein the glucose-induced genes comprise glucose regulated proteins, glycosylated proteins, glycolytic enzymes, genes as recited in Table 3, or combinations thereof.

15. A method according to claim 13, wherein the hypoxia-induced genes comprise heat shock proteins, genes for nitric oxide synthases, genes for matrix metalloproteins, anti-apoptotic genes, pro-apoptotic genes, genes for cyclooxygenases, genes for growth factors, genes for hypoxia-induced factors, genes involved in the synthesis of cytokines, chemokines, adhesion molecules, or combinations thereof.

16. A method according to claim 13, wherein the acidosis-induced genes comprise of the genes recited in Table 2, the genes recited in Table 3, or combinations thereof.

17. A method according to claim 13, wherein the ischemia-induced genes comprise the genes recited in Table 3 or combinations thereof.

18. A method according to claim 14, wherein the glycolytic enzymes comprise aldolase-A, lactate dehydrogenase-A, phosphofructokinase-L, pyruvate kinase-M, hypoxia-inducible factor, or combinations thereof.

19. A method according to claim 12, wherein the heat shock proteins comprise ubiqutin, HSP10, HSP27, HSP25, HSP32, HSP47, HSP60, HSC70, HSP70, HSP90, HSP100/105, or combinations thereof.

20. A method according to claim 1, wherein the injury database comprises organ specific injury database, disease specific injury database, or combinations thereof.

21. A method according to claim 20, wherein the organ specific injury database includes brain injury database, spinal cord injury database, blood injury database, muscle injury database, nerve injury database, lung injury database, liver injury database, heart injury database, kidney injury database, genitalia injury database, eye injury database, ear injury database, nose injury database, teeth injury database, bone injury database, white blood cell injury database, endocrine gland injury database, gastrointestinal injury database, blood vessel injury database, or combinations thereof.

22. A method according to claim 20, wherein the disease specific injury database comprises global ischemic injury database, focal ischemic profile, status epilepticus injury database, hypoxia injury database, hypoglycemia injury database, cerebral hemorrhage injury database, hemorrhage injury database for one or more organs, diabetes complications injury database, psychosis injury database, psychiatric disease injury database, bipolar injury database, schizophrenia injury database, headache injury database, acute migraine headache injury database, endocrine disease injury database, uremia injury database, injury database for ammonemia with hepatic failure, toxin overdose injury database, drug overdose injury database, Alzheimer's disease injury database, Parkinson's disease injury database, Tourettes disease injury database, muscle disease injury database, proliferative disease injury database, neurofibromatosis injury database, nerve disease injury database, other dementing illness injury database, inflammatory diseases injury database, autoimmune diseases injury database, infectious diseases injury database, demyelinating diseases injury database, trauma injury database, tumors injury database, cancer injury database, degenerative and metabolic diseases including Alzheimer's injury database, genetic or familial diseases injury database, or combinations thereof.

23. A method according to claim 1, wherein the injury assessment comprises movement disorder injury assessment.

24. A method according to claim 1, wherein the injury assessment comprises genetic disorder injury assessment using a single blood sample.

25. A method according to claim 1, wherein the injury assessment comprises psychosis injury assessment.

26. A method according to claim 1, wherein the injury assessment comprises headache injury assessment.

27. A method according to claim 1, wherein the injury assessment comprises organ injury assessment.

28. A method according to claim 1, wherein the injury assessment comprises brain injury assessment.

29. A method according to claim 1, wherein the injury assessment comprises stroke injury assessment.

30. A method according to claim 1, wherein the injury assessment comprises seizure injury assessment.

31. A method according to claim 1, wherein the injury assessment comprises hypoglycemia injury assessment.

32. A method according to claim 1, wherein the injury assessment comprises hypoxia injury assessment.

33. A method according to claim 1, wherein the injury assessment comprises diabetes assessment.

34. A method according to claim 1, wherein the injury assessment comprises infectious disease assessment.

35. A method according to claim 1, wherein the injury assessment comprises immune mediated disease assessment.

36. A method according to claim 1, wherein the injury assessment comprises efficacy or toxicity assessment, or a combination thereof.

37. A method according to claim 1, wherein the injury assessment comprises proliferative disease assessment.

38. A method of stroke injury assessment in an individual comprising the steps of:
a. obtaining a peripheral blood sample from the individual,
b. capturing a pattern of expression,
c. defining the pattern of expression, and
d. comparing the pattern of expression to an injury database to assess stroke injury.

39. A method according to claim 38, wherein the pattern of expression comprises patterns of gene expression, protein expression, or combinations thereof.

40. A method according to claim 38, wherein the injury database comprises genomic injury database, proteomic injury database, or combinations thereof.

41. A method according to claim 38, wherein the stroke injury comprises ischemic, hemorrhagic stroke, or combinations thereof.

42. A method according to claim 39, wherein the genes in the pattern of gene expression comprise hypoxia-induced genes, glucose-induced genes, or combinations thereof.

43. A method of hypoxia injury assessment in an individual comprising the steps of:
a. obtaining a peripheral blood sample from the individual,
b. capturing a pattern of expression,
c. defining the pattern of expression, and
d. comparing the pattern of expression to an injury database to assess hypoxia injury.

44. A method according to claim 43, wherein the pattern of expression comprises patterns of gene expression, protein expression, or combinations thereof.

45. A method according to claim 43, wherein the injury database comprises genomic injury database, proteomic injury database, or combinations thereof.

46. A method according to claim 44, wherein the genes in the pattern of gene expression comprise glucose-induced genes, hypoxia-induced genes, acidosis-induced genes, ischemia-induced genes, or combinations thereof.

47. A method of hypoglycemia injury assessment in an individual comprising the steps of:
a. obtaining a peripheral blood sample from the individual,
b. capturing a pattern of expression,
c. defining the pattern of expression, and
d. comparing the pattern of expression to an injury database to assess hypoglycemia injury.

48. A method according to claim 47, wherein the pattern of expression comprises patterns of gene expression, protein expression, or combinations thereof.

49. A method according to claim 47, wherein the injury database comprises genomic injury database, proteomic injury database, or combinations thereof.

50. A method according to claim 48, wherein the genes in the pattern of gene expression comprise glucose-induced genes.

51. A method of seizure injury assessment in an individual comprising the steps of:
a. obtaining a peripheral blood sample from the individual,
b. capturing a pattern of expression,
c. defining the pattern of expression, and
d. comparing the pattern of expression to an injury database to assess seizure injury.

52. A method according to claim 51, wherein the pattern of expression comprises patterns of gene expression, protein expression, or combinations thereof.

53. A method according to claim 51, wherein the injury database comprises genomic injury database, proteomic injury database, or combinations thereof.

54. A method according to claim 51, wherein the seizure injury comprises status epilepticus, single tonic-clonic seizure, syncope, pseudo-seizure, or combinations thereof.

55. A method according to claim 52, wherein the genes in the pattern of gene expression comprise histamine H2-receptor, c-jun leucine zipper interactive protein, Glut3, the vesicular monoamine transporter, TNF intracellular domain interacting protein, vascular tyrosine phosphatase, or combinations thereof.

56. A method of movement disorder injury assessment in an individual comprising the steps of:
a. obtaining a peripheral blood sample from the individual,
b. capturing a pattern of expression,
c. defining the pattern of expression, and
d. comparing the pattern of expression to an injury database to assess movement disorder injury.

57. A method according to claim 56, wherein the pattern of expression comprises patterns of gene expression, protein expression, or combinations thereof.

58. A method according to claim 56, wherein the injury database comprises genomic injury database, proteomic injury database, or combinations thereof.

59. A method according to claim 56, wherein the movement disorder injury comprises Parkinson's, Huntington's disease, Tourettes, Sydenhams Chorea, Diffuse Lewy Body Disease, Corticobasal ganglionic disease, or combinations thereof.

60. A method according to claim 59, wherein the movement disorder injury is Parkinson's disease.

61. A method according to claim 59, wherein the movement disorder injury is Tourettes.

62. A method according to claim 60, wherein the genes in the pattern of gene expression comprise SEQ ID NO:1, SEQ ID NO:2, or combinations thereof.

63. A method of diabetes injury assessment in an individual comprising the steps of:
a. obtaining a peripheral blood sample from the individual,
b. capturing a pattern of expression,
c. defining the pattern of expression, and
d. comparing the pattern of expression to an injury database to assess diabetes injury.

64. A method according to claim 63, wherein the pattern of expression comprises patterns of gene expression, protein expression, or combinations thereof.

65. A method according to claim 63, wherein the injury database comprises genomic injury database, proteomic injury database, or combinations thereof.

66. A method of infectious disease assessment in an individual comprising the steps of:
a. obtaining a peripheral blood sample from the individual,
b. capturing a pattern of expression,
c. defining the pattern of expression, and
d. comparing the pattern of expression to an injury database to assess infectious disease.

67. A method according to claim 66, wherein the pattern of expression comprises patterns of gene expression, protein expression, or combinations thereof.

68. A method according to claim 66, wherein the injury database comprises genomic injury database, proteomic injury database, or combinations thereof.

69. A method according to claim 66, wherein the infectious disease comprises tuberculosis, viral, prion or combinations thereof.

70. A method of immune mediated disease assessment in an individual comprising the steps of:
a. obtaining a peripheral blood sample from the individual,
b. capturing a pattern of expression,
c. defining the pattern of expression, and
d. comparing the pattern of expression to an injury database to assess immune mediated disease.

71. A method according to claim 70, wherein the pattern of expression comprises patterns of gene expression, protein expression, or combinations thereof.

72. A method according to claim 70, wherein the injury database comprises genomic injury database, proteomic injury database, or combinations thereof.

73. A method according to claim 70, wherein the immune mediated disease comprises Graves, Rheumatoid arthritis, Thyroiditis/hypothyroidism, Vitiligo, IDDM, Multiple sclerosis, Primary glomerulonephritis, Systemic lupus erythematosus, Sjogren's, asthma, transplant rejection or combinations thereof.

74. A method of efficacy or toxicity assessment in an individual comprising the steps of:
a. obtaining a peripheral blood sample from the individual,
b. capturing a pattern of expression,
c. defining the pattern of expression, and
d. comparing the pattern of expression to an injury database to assess efficacy or toxicity.

75. A method according to claim 74, wherein the pattern of expression comprises patterns of gene expression, protein expression, or combinations thereof.

76. A method according to claim 74, wherein the injury database comprises genomic injury database, proteomic injury database, or combinations thereof.

77. A method of psychosis assessment in an individual comprising the steps of:
a. obtaining a peripheral blood sample from the individual,
b. capturing a pattern of expression,
c. defining the pattern of expression, and
d. comparing the pattern of expression to an injury database to assess the psychosis.

78. A method according to claim 77, wherein the pattern of expression comprises patterns of gene expression, protein expression, or combinations thereof.

79. A method according to claim 77, wherein the injury database comprises genomic injury database, proteomic injury database, or combinations thereof.

80. A method according to claim 77, wherein the psychosis is schizophrenia.

81. A method according to claim 77, wherein the psychosis is bipolar.

82. A method of headache assessment in an individual comprising the steps of:
a. obtaining a peripheral blood sample from the individual,
b. capturing a pattern of expression,
c. defining the pattern of expression, and
d. comparing the pattern of expression to an injury database to assess headache injury.

83. A method according to claim 82, wherein the pattern of expression comprises patterns of gene expression, protein expression, or combinations thereof.

84. A method according to claim 82, wherein the injury database comprises genomic injury database, proteomic injury database, or combinations thereof.

85. A method according to claim 82, wherein the headache is an acute migraine headache.

86. A method of genetic disorder injury assessment in an individual comprising the steps of:
a. obtaining a peripheral blood sample from the individual,
b. capturing a pattern of expression,
c. defining the pattern of expression, and
d. comparing the pattern of expression to an injury database to assess genetic disorder injury.

87. A method according to claim 86, wherein the pattern of expression comprises patterns of gene expression, protein expression, or combinations thereof.

88. A method according to claim 86, wherein the injury database comprises genomic injury database, proteomic injury database, or combinations thereof.

89. A method according to claim 86, wherein the genetic disorder injury is neurofibromatosis.

90. A method of proliferative disease injury assessment in an individual comprising the steps of:
a. obtaining a peripheral blood sample from the individual,
b. capturing a pattern of expression,
c. defining the pattern of expression, and
d. comparing the pattern of expression to an injury database to assess proliferative disease injury.

91. A method according to claim 90, wherein the pattern of expression comprises patterns of gene expression, protein expression, or combinations thereof.

92. A method according to claim 90, wherein the injury database comprises genomic injury database, proteomic injury database, or combinations thereof.

93. A method according to claim 90, wherein the proliferative disease injury is neurofibromatosis.
